(19) United States
(12) Patent Application Publication
(10) Pub. No.: US 2025/0221997 A1
(43) Pub. Date: Jul. 10, 2025

(54) TYK2 INHIBITOR FORMULATIONS AND METHODS OF MAKING THE SAME

(71) Applicant: Takeda Pharmaceutical Company Limited, Osaka (JP)

(72) Inventors: Alan John COLLIS, Lexington, MA (US); Stuart LEVY, Arlington, MA (US); Colin MINCHOM, Arlington, MA (US); Andrew PHIMISTER, El Cerrito, CA (US); Sarah Jean BETHUNE, Fruit Heights, UT (US); David Shank FRY, Loveland, CO (US); Erica SCHLESINGER, Sisters, OR (US); Dan SMITHEY, Bend, OR (US); Randy WALD, Bend, OR (US); James FENNEWALD, Bend, OR (US)

(21) Appl. No.: 18/850,495

(22) PCT Filed: Mar. 24, 2023
(86) PCT No.: PCT/US2023/064899

§ 371 Date: Sep. 24, 2024

Related U.S. Application Data
(60) Provisional application No. 63/269,945, filed on Mar. 25, 2022.

(51) Int. Cl. A61K 31/519; A61K 9/16

(52) U.S. Cl. CPC A61K 31/519; A61K 9/1611; A61K 9/1617; A61K 9/1623; A61K 9/1635; A61K 9/1652

(57) ABSTRACT

Pharmaceutical compositions and unit dosage forms can include a TYK2 inhibitor, methods of making the same. Methods of treatment can include administering the same to a patient.

### PRIORITY CLAIMS

This application claims priority from U.S. Provisional Patent Application No. 63/269,945, filed Mar. 25, 2022, which is incorporated by reference in its entirety.

### TECHNICAL FIELD

The present invention relates to pharmaceutical compositions of a tyrosine-protein kinase 2 (TYK2) inhibitor and to methods of making the compositions. The invention also provides methods of treating disorders using the pharmaceutical compositions.

### BACKGROUND

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signalling by effecting a phosphoryl transfer from a nucleoside triphosphate to a protein acceptor that is involved in a signalling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxins, and H₂O₂), cytokines (e.g., interleukin-1 (IL-1), interleukin-8 (IL-8), and tumour necrosis factor a (TNF-a)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by kinase-mediated events. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases.

TYK2 catalyzes the phosphorylation of STAT proteins downstream of a number of cytokine receptors, including the Type I interferon receptor and the IL-12 and IL-23 receptors. The activation of TYK2-dependent receptors by their cytokine ligands results in the activation of STAT-dependent transcription and cellular functional responses specific for the receptors and cell types on which they are expressed. The cytokine signalling pathways regulated by TYK2 play key roles in several immune-mediated disorders. The cytokine IL-12 is essential for the development of Type 1 T-helper cells (Th1) which produce interferon-gamma, a major effector molecule in systemic autoimmune disorders such as systemic lupus erythematosus. The cytokine IL-23 is central for the expansion and survival of Th17 cells and innate lymphoid cells, both of which have been shown to play key pathogenic roles in autoimmunity. IL-23 stimulation drives the production of key proinflammatory cytokines by Th17 cells, including IL-17A, IL-17F, and IL-22, all of which are effector molecules important for pathogenesis of conditions such as psoriasis, psoriatic arthritis, and spondylarthritis. Inhibition of TYK2 would be expected to impact multiple immune-mediated disorders through its effects on the IL-23/Th17/Th22 axis, IL-12-mediated Th1 functions, and Type I interferon-driven modulation of diverse immune pathways and cell types.

### SUMMARY

In one aspect, the present disclosure provides pharmaceutical compositions, formulations, and unit dosage forms including:
(a) Compound 1:

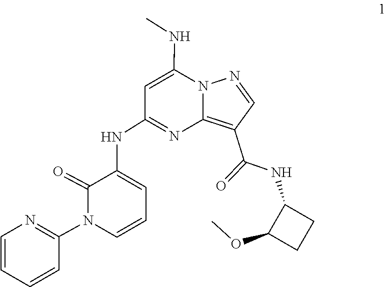 [SMILES: CNc1cc(Nc2cccn(-c3ccccn3)c2=O)nc2c(C(=O)N[C@@H]3CC[C@H]3OC)cnn12]

or a solvate or a pharmaceutically acceptable salt thereof;
(b) one or more fillers or diluents (e.g., microcrystalline cellulose);
(c) one or more solubilizers (e.g., D-α-tocopherol polyethylene glycol succinate [Vitamin E TPGS]); and
(d) one or more binders (e.g., povidone).

In another aspect, the disclosure provides a method of making a unit dosage form including:
(a) Compound 1:

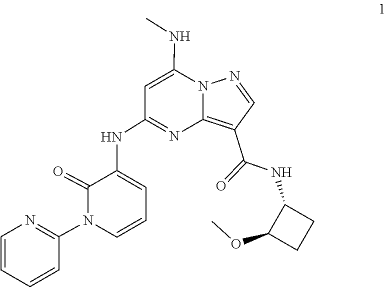 [SMILES: CNc1cc(Nc2cccn(-c3ccccn3)c2=O)nc2c(C(=O)N[C@@H]3CC[C@H]3OC)cnn12]

or a solvate or a pharmaceutically acceptable salt thereof;
(b) one or more fillers or diluents (e.g., microcrystalline cellulose);
(c) one or more solubilizers (e.g., D-α-tocopherol polyethylene glycol succinate [Vitamin E TPGS]); and
(d) one or more binders (e.g., povidone).

In another aspect, pharmaceutical compositions, formulations, and unit dosage forms can include:
(a) Compound 1:

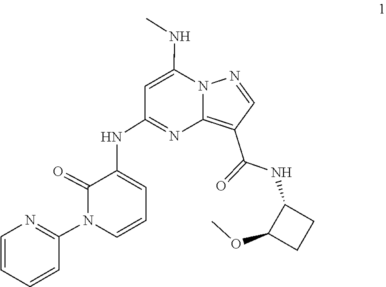 [SMILES: CNc1cc(Nc2cccn(-c3ccccn3)c2=O)nc2c(C(=O)N[C@@H]3CC[C@H]3OC)cnn12]

or a solvate thereof, or a pharmaceutically acceptable salt thereof;
   wherein Compound 1 is in the form of a spray-dried dispersion (SDD).

In some embodiments, the SDD can include a cellulosic polymer such as hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate, or carboxymethyl ethyl cellulose. In some embodiments, the SDD can include a cellulosic polymer that is at least partially ionized at physiologically relevant pHs, for example, hydroxypropyl methyl cellulose acetate succinate, hydroxypropyl methyl cellulose phthalate, cellulose acetate phthalate, cellulose acetate trimellitate, or carboxymethyl ethyl cellulose. In some embodiments, the SDD can include hydroxypropyl methyl cellulose acetate succinate (HPMCAS). In some embodiments, the SDD can include HPMC-AS-M (hydroxypropylmethylcellulose, acetate succinate, Grade M; also known as Hypromellose acetate succinate).

In some embodiments, the SDD can be a spray-dried about 2:1 to about 1:2 (w/w) mixture of Compound 1 with HPMC-AS-M.

In some embodiments, the pharmaceutical composition, formulation, or unit dosage form can include:
a) about 1-4 mg of Compound 1 or a solvate or a pharmaceutically acceptable salt thereof, optionally as an SDD such as one of those described herein;
b) about 35-140 mg of a first filler such as mannitol;
c) optionally, about 35-140 mg of a second filler such as microcrystalline cellulose; d) about 4-18 mg of a disintegrant such as croscarmellose sodium;
e) about 0.5-3.0 mg of a glidant such as colloidal silicon dioxide;
f) about 0.4-2.0 mg of a lubricant such as sodium stearyl fumarate; and
g) optionally, about 0.5-2 mg of a wetting agent such as sodium lauryl sulfate.

In some embodiments, the pharmaceutical composition, formulation, or unit dosage form can include:
a) about 2.5-10 mg of Compound 1 or a solvate or a pharmaceutically acceptable salt thereof, optionally as an SDD such as one of those described herein;
b) about 30-130 mg of a first filler such as mannitol;
c) optionally, about 30-130 mg of a second filler such as microcrystalline cellulose; d) about 4-20 mg of a disintegrant such as croscarmellose sodium;
e) about 0.5-4.0 mg of a glidant such as colloidal silicon dioxide;
f) optionally, about 0.5-4.0 mg of a wetting agent such as sodium lauryl sulfate; and
g) about 0.4-2.5 mg of a lubricant such as sodium stearyl fumarate.

In some embodiments, the pharmaceutical composition, formulation, or unit dosage form can include:
a) about 7.5-30 mg of Compound 1 or a solvate or a pharmaceutically acceptable salt thereof, optionally as an SDD such as one of those described herein;
b) about 25-130 mg of a first filler such as mannitol;
c) optionally, about 25-130 mg of a second filler such as microcrystalline cellulose;
d) about 4-20 mg of a disintegrant such as croscarmellose sodium;
e) about 0.5-4.0 mg of a glidant such as colloidal silicon dioxide;
f) optionally, about 0.5-4.0 mg of a wetting agent such as sodium lauryl sulfate; and
g) about 0.4-4.0 mg of a lubricant such as sodium stearyl fumarate.

In some embodiments, the pharmaceutical composition, formulation, or unit dosage form can include:
a) about 12.5-50 mg of Compound 1 or a solvate or a pharmaceutically acceptable salt thereof, optionally as an SDD such as one of those described herein;
b) about 20-95 mg of a first filler such as mannitol;
c) optionally, about 20-95 mg of a second filler such as microcrystalline cellulose;
d) about 4-20 mg of a disintegrant such as croscarmellose sodium;
e) about 0.5-4.0 mg of a glidant such as colloidal silicon dioxide;
f) optionally, about 0.5-4.0 mg of a wetting agent such as sodium lauryl sulfate; and
g) about 0.4-4.0 mg of a lubricant such as sodium stearyl fumarate.

In some embodiments, the pharmaceutical composition, formulation, or unit dosage form can include:
a) about 25-75 mg of Compound 1 or a solvate or a pharmaceutically acceptable salt thereof, optionally as an SDD such as one of those described herein;
b) about 24-96 mg of a first filler such as mannitol;
c) optionally, about 24-96 mg of a second filler such as microcrystalline cellulose;
d) about 6-25 mg of a disintegrant such as croscarmellose sodium;
e) about 1.0-5.0 mg of a glidant such as colloidal silicon dioxide;
f) optionally, about 1.0-5.0 mg of a wetting agent such as sodium lauryl sulfate; and
g) about 0.7-2.6 mg of a lubricant such as sodium stearyl fumarate.

In some embodiments, the pharmaceutical composition, formulation, or unit dosage form can include one of the following % w/w of ingredients shown below.

| | 2 mg | 5 mg | | 15 mg |
|---|---|---|---|---|
| Ingredients | w/w % | w/w % | w/w % | w/w % |
| Spray Dried | 1-5 | 2.5-10.0 | 2.5-10.0 | 7.0-40.0 |
| Intermediate | | | | |
| Microcrystalline | 0-80 or 18.0- | 0-75.0 or 18.0- | 0-75.0 or 18.0- | 0-70.0 or 18.0- |
| Cellulose | 80.0 | 75.0 | 75.0 | 70.0 |
| Mannitol | 18.0-80.0 | 18.0-75.0 | 18.0-75.0 | 18.0-70.0 |
| Croscarmellose | 4.5-26.0 | 5.0-18.0 | 6.5-27.0 | 4.6-20.0 |
| Sodium | | | | |
| Colloidal Silicon | 0.25-2.0 | 0.25-2.0 | 0.25-2.0 | 0.5-2.2 |
| Dioxide | | | | |
| Sodium Lauryl | 0.0-2 | 0.5-2 | 0.0-2.0 | 0.0-2.0 |
| Sulfate | | | | |
| Sodium Stearyl | 0.2-1.5 | 0.1-0.5 | 0.25-1.0 | 0.1-0.5 |
| Fumarate | | | | |

The Spray Dried Intermediate can include a spray-dried solid of Compound 1 and a cellulosic polymer, such as a 1.5:1 to 1:1.5 w/w ratio of Compound 1: HPMC-AS (hydroxypropylmethylcellulose-acetate, succinate).

| | 25 mg | | 50 mg |
|---|---|---|---|
| Ingredients | w/w % | w/w % | w/w % |
| Spray Dried | 23.0-52.0 | 16.0-66.0 | 20.0-80.0 |
| Intermediate | | | |
| Microcrystalline | 0-70.0 or | 0-60.0 or | 0-58.0 or |
| Cellulose | 17.0-70.0 | 16.5-60.0 | 12.5-58.0 |
| Mannitol | 12.5-51.0 | 12.0-48.0 | 8.0-38.0 |
| Croscarmellose Sodium | 4.9-20.0 | 4.0-16.0 | 4.9-20.0 |
| Colloidal Silicon | 0.5-2.0 | 0.5-2.0 | 0.5-2.0 |
| Dioxide | | | |
| Sodium Lauryl Sulfate | 0.5-2.0 | 0.0-2.0 | 0.5-2.0 |
| Sodium Stearyl Fumarate | 0.3-1.5 | 0.3-1.5 | 0.3-1.5 |

The Spray Dried Intermediate can include a spray-dried solid of Compound 1 and a cellulosic polymer, such as a 1.5:1 to 1:1.5 w/w ratio of Compound 1: HPMC-AS (hydroxypropylmethylcellulose-acetate, succinate).

### BRIEF DESCRIPTION OF THE FIGURES

FIG. 1 is a graph depicting dissolution of Compound 1 formulations in aqueous media. The “0.1 N HCl” curve indicates the dissolution % of a blend of crystalline Compound 1 with microcrystalline cellulose (MCC), while the other two curves indicate dissolution % of a granulation of crystalline Compound 1 with either 5% or 20% Vitamin E TPGS and MCC.

FIG. 2 is a flow chart depicting a formulation process that includes wet granulation of Compound 1 with MCC (microcrystalline cellulose, diluent, Avicel PH101), Vitamin E TPGS (solubility enhancer), PVP K30 (polyvinylpyrrolidone, binder), and water. After granulation the wet granules are dried in a fluid bed dryer until LOD≤1.5%, passed through a Comil to break up any aggregates, and blended to provide the bulk capsule fill material. The Compound 1 5 mg capsules are filled using an Xcelodose system based on weight of input material. The Compound 1 25 mg capsules are filled used a Profill system based on volume. Content unifonnity tests are conducted after capsule filling. Both the 5 mg and 25 mg strength capsules are filled in size #2 hard shell HPMC based capsules. Filled capsules are packaged 12 count each into 60 cc HPDE bottles.

FIG. 3 is a graph depicting an XRPD spectrum of a Form C polymorph of Compound 1.

FIG. 4 is a flow chart depicting a formulation process for a spray-dried dispersion (SDD) formulation.

FIG. 5 is a graph depicting an XRPD spectrum of Form A of Compound 1.

### DETAILED DESCRIPTION

### Compound 1

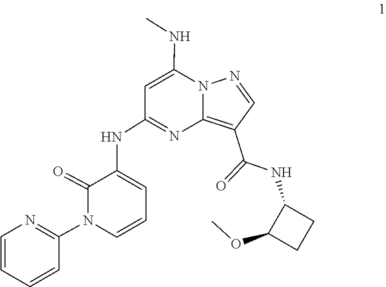 [SMILES: CNc1cc(Nc2cccn(-c3ccccn3)c2=O)nc2c(C(=O)N[C@@H]3CC[C@H]3OC)cnn12]

is a TYK2 inhibitor previously disclosed by Applicant. See U.S. Pat. No. 11,046,698. Solubilization of the compound is problematic and may present barriers in formulating the compound for patient administration or treatment of said patients because, e.g., solubility may affect bioavailability and therapeutic outcomes. The present disclosure satisfies the need for solubilizing formulations of Compound 1 and provides other related advantages.

Compound 1 is an oral, allosteric, and selective TYK2 inhibitor that is being investigated for the treatment of psoriasis, psoriatic arthritis and other inflammatory and autoimmune diseases. There are currently no TYK2 inhibitors approved, and this compound is the most selective TYK2 inhibitor currently in clinical development. Furthermore, Compound 1's selectivity and potential to provide higher levels of TYK2 inhibition for a longer period with once-daily (QD) dosing may confer clinical and ultimately commercial advantages over other TYK2 inhibitors in development. TYK2 is a member of the Janus kinase (JAK) family of kinases, a class of intracellular signalling proteins that regulate chronic inflammation in inflammatory and autoimmune diseases. Although JAK inhibition can be effective in treating inflammatory and autoimmune diseases, it also can produce on-target safety issues by modulating a broad variety of cytokine pathways. As a result, while JAK inhibitors have become established oral treatments for numerous inflammatory and autoimmune diseases, their clinical utility is constrained by elevated risk of infections and other side effects that have resulted in U.S. Food and Drug Administration (FDA)-mandated boxed warnings and dosing limitations as part of their labelling. Designing selective JAK inhibitors in an optimized formulation for in vivo, effective dissolution that directly and specifically inhibit the intended kinase function is challenging due to the structural similarity between the catalytic (orthosteric or JH1) sites for drug targeting on the JAK catalytic domains and due to specific physical properties (e.g., solubility) of JAK inhibitors like Compound 1. Based on human genetic data and growing clinical evidence for the selectivity of allosteric TYK2 inhibitors, the present approach of selective allosteric inhibition of TYK2 provides an optimal balance of achieving strong efficacy while potentially avoiding safety concerns associated with broader JAK inhibition for the treatment of multiple inflammatory and autoimmune diseases. Furthermore, the present approach of formulation of the inhibitor facilitates the dissolution of the compound when used in treatment requiring the aforementioned pharmacotherapy.

Formulations and methods described herein include those described generally above, and are further illustrated by the classes, subclasses, and species disclosed herein. As used herein, the following definitions shall apply unless otherwise indicated. For purposes of the present disclosure, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry, 5^{th} Ed., Ed.: Smith, M. B. and March, J., John Wiley & Sons, New York: 2001, the entire contents of which are hereby incorporated by reference.

“Avicel® PH-101,” as used herein refers to cellulose of formula III:

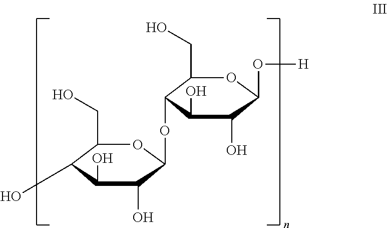

wherein n is between about 5 and about 2,000. In some embodiments, n is between about 50 and 500.

“PVP30,” as used herein, refers to a compound of formula II:

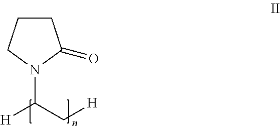

wherein n is about 200 to about 1000. In some embodiments n is about 250 to about 500.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In some embodiments, the term “aryl” used alone or as part of a larger moiety as in “aralkyl,” “aralkoxy,” or “aryloxyalkyl,” refers to monocyclic and bicyclic ring systems having a total of five to 10 ring members, wherein at least one ring in the system is aromatic and wherein each ring in the system contains three to seven ring members. In certain embodiments of the compounds, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned for the compounds herein are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen, —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5- to 6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5 to 6-membered saturated, partially unsaturated, or aryl ring having 0-4 heteroatoms independently selected from nitrogen, oxygen, and sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5- to 6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, and sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, mesylate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

As used herein, “about” or “approximately” in reference to a numerical value means that the stated numerical value may vary by up to 10% of the stated value. For example, “about 10” refers to a value of 9.9 to 10.1 (10+/−0.1).

The present disclosure contains embodiments which are stated to “comprise” items. The disclosure also contemplates analogous embodiments which “consist essentially of” or “consist of” said items.

“Nominal particle size,” as used herein refers to a particle size representing the mass average of the particles.

“Loose bulk density,” as used herein, refers to the mass per unit volume of a loose powder.

A “solubilizer,” as used herein, may refer to one or more compounds added to the formulation that increases solubility in any given solvent system, e.g., water, organic solvents, biological fluids, simulated biological fluids, and mixtures thereof.

A “binder,” as used herein, may refer to one or more compounds that promote cohesiveness among the other components in the formulation, the binders, also called adhesives, help the other ingredients in a formulation to mix together. Tablet binders may help turn powder to granules; this may be achieved during the process of granulation.

A “surfactant,” as used herein, may refer to one or more compounds that lower the surface tension between two liquids, between a gas and a liquid, or between a liquid and a solid. Surfactants may act as detergents, wetting agents, emulsifiers, foaming agents, or dispersants.

“Glidants,” as used herein, are additive substances that are used to enhance the flowability of a powder by reducing the interparticle friction, surface charge, and cohesion, which in turn decreases the angle of repose. They are often incorporated as a dry powder just prior to direct compression.

“Granulation,” as used herein, refers to a process to produce larger or smaller granules or particles of a substance or mixture of substances. The process also may remove fine granules and improve flowability within the formulation. Both wet granulation and/or dry granulation may be employed. Dry granulation is achieved using only a combination of granules without the need for any liquid thereon. Slugging uses a tablet press to form large tablets that vary in weight due to the poor flowability of the formulation. The slugs created are then put through a granulator to be broken down into granules and then compressed once again for a final granulated product.

The process may include roller compaction wherein the formulation is fed through a roller compactor, the mixture is fed through a top hopper where two rollers would compact the powder to form a ribbon, this would then pass through a granulator where the powder is forced against a mesh which creates the granules of the desired size. In some embodiments, the granules have a mean particle diameter between about 0.01 mm and about 10 mm. In some embodiments, the granules have a mean particle diameter between about 0.2 mm and about 0.4 mm.

Wet granulation may be used to facilitate aggregation of particles. An adhesive usually called a binder may be incorporated in the form of a solution or suspension in a suitable liquid.

A fluid bed granulator may be used for wet granulation wherein hot air is fed into a bed lifting the granules whilst they are sprayed, granules bind together to form bridges between themselves. The initial phase of wet granulation may include the spraying of the granules, the spray rate may be varied. Wetting of the powder may then begin to form liquid bridges between the granules. The process is continued until the required size granule is formed with solid bridges between the granules. Once complete granules may go to the drying process, the spraying process may be turned off and the hot air will continue to flow through the bed drying the granules. The wet granulation may be performed in a high shear wet granulator, commercially available or known to those having ordinary skill in the art, e.g., a wet granulator as described in Liu et al., “A review of high shear wet granulation for better process understanding, control and product development,” Powder Technology, 381, March 2021, Pages 204-223, the entire disclosure of which is hereby incorporated by reference. As to the drying process, such methods and apparatuses to perform the same will be known to those having ordinary skill in the art. The drying may be performed in a fluid bed dryer, e.g., as described in Srivastava et al., “Fluid Bed Technology: Overview and Parameters for Process Selection,” International Journal of Pharmaceutical Sciences and Drug Research 2010; 2(4): 236-246, the entire disclosure of which is hereby incorporated by reference.

Screen milling of the process material may be used, e.g., for example after drying. Screen milling procedures and equipment are will known to those having ordinary skill in the art, e.g., as described in Kotamarthy, L. V. G., (2018). “Understanding the effect of granulation and mill process parameters on granule critical quality attributes,” [Master's dissertation, Rutgers University]. Rutger's Library, the entire disclosure of which is hereby incorporated by reference.

“Blending,” as used herein, refers to techniques for blending solid material well known to those of ordinary skill in the art. Such techniques and machinery for blending are described in the art, e.g., Mendez et al., “Evaluation of powder mixing operation during batch production: application to operational qualification procedure in the pharmaceutical industry.” Powder Technol. 2009; 198:310-3. Hamby N. “An engineering view of pharmaceutical powder mixing.” Pharm Sci Technol Today. 2000; 3:303-9. Remy B. et al., “Discrete element simulation of free flowing grains in a four-bladed mixer.” AIChE J. 2009; 55:2035-48; and Stieß M. “Mechanische Verfahrenstechnik-Partikeltechnologie 1.” 3rd ed. Berlin: Springer; 2009, the entire disclosures each of which are hereby incorporated by reference in their entireties.

Other methods of granulation are known to those having ordinary skill in the art, and are described in Miyazaki S et al. “Drug release from oral mucosal adhesive tablets of chitosan and sodium alginate.” Int J Pharm 1995; 118: 257-263; Selmeczi B. “The influence of the compressional force on the physical properties of tablets made by different technological processes.” Arch Pharm (Weinheim) 1974; 307(10): 755-760; and Beg et al., 2021, “Handbook of Analytical Quality by Design,” Elsevier Inc., the entire disclosures each of which are hereby incorporated by reference in their entireties.

“De-lumping,” as used herein, refers to a process by passing the powder formulation through a mesh screen. Screens used herein for processes, e.g., blending or de-lumping, may have a mesh size between about 1 to about 100 μm, about 100 to about 500 m, about 500 to about 1,000 m, about 1,000 to about 5,000 m, about 5,000 m to about 10,000 m, about 10,000 to about 50,000 μm, or about 50,000 to about 100,000 μm.

In one aspect, the present disclosure provides pharmaceutical compositions, formulations, and unit dosage forms can include:
(a) Compound 1:

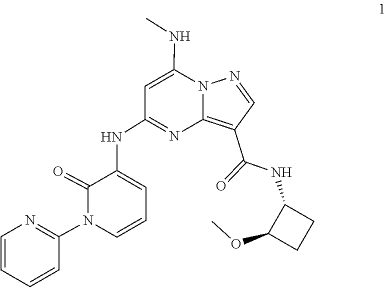 [SMILES: CNc1cc(Nc2cccn(-c3ccccn3)c2=O)nc2c(C(=O)N[C@@H]3CC[C@H]3OC)cnn12]

or a solvate thereof, or a pharmaceutically acceptable salt thereof;
(b) one or more fillers or diluents;
(c) one or more solubilizers;
(d) one or more binders;
(e) optionally, one or more glidants;
(f) optionally, one or more lubricants; and
(g) optionally, one or more surfactants.

In one aspect, the present disclosure provides pharmaceutical compositions, formulations, and unit dosage forms comprising:
(a) Compound 1:

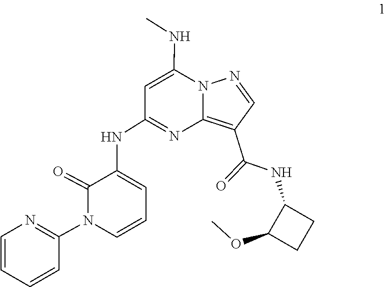 [SMILES: CNc1cc(Nc2cccn(-c3ccccn3)c2=O)nc2c(C(=O)N[C@@H]3CC[C@H]3OC)cnn12]

or a solvate thereof, or a pharmaceutically acceptable salt thereof;
(b) one or more fillers or diluents;
(c) one or more solubilizers; and
(d) one or more binders.

In one aspect, the present disclosure provides pharmaceutical compositions, formulations, and unit dosage forms consisting essentially of:
(a) Compound 1:

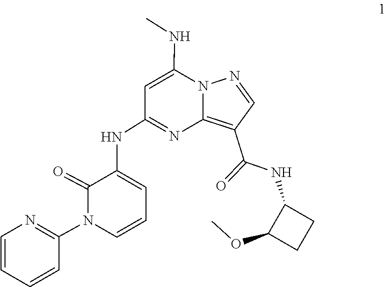 [SMILES: CNc1cc(Nc2cccn(-c3ccccn3)c2=O)nc2c(C(=O)N[C@@H]3CC[C@H]3OC)cnn12]

or a solvate thereof, or a pharmaceutically acceptable salt thereof;
(b) one or more fillers or diluents;
(c) one or more solubilizers; and
(d) one or more binders.

In one aspect, the present disclosure provides pharmaceutical compositions, formulations, and unit dosage forms consisting of:
(a) Compound 1:

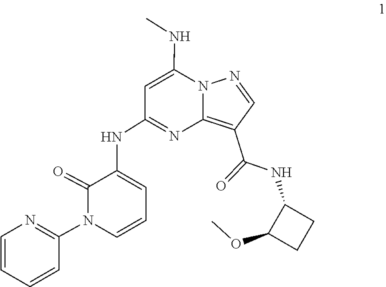 [SMILES: CNc1cc(Nc2cccn(-c3ccccn3)c2=O)nc2c(C(=O)N[C@@H]3CC[C@H]3OC)cnn12]

or a solvate thereof, or a pharmaceutically acceptable salt thereof;
(b) one or more fillers or diluents;
(c) one or more solubilizers; and
(d) one or more binders.

In one aspect, the present disclosure provides pharmaceutical compositions, formulations, and unit dosage forms comprising:
(a) Compound 1:

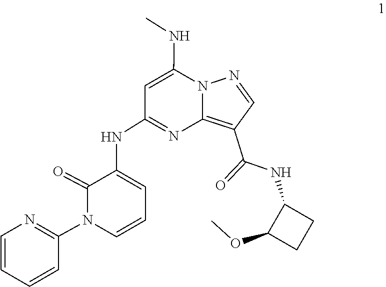 [SMILES: CNc1cc(Nc2cccn(-c3ccccn3)c2=O)nc2c(C(=O)N[C@@H]3CC[C@H]3OC)cnn12]

or a solvate thereof, or a pharmaceutically acceptable salt thereof;
(b) one or more fillers or diluents;
(c) one or more solubilizers of formula I:

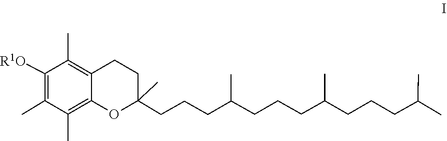

or a pharmaceutically acceptable salt thereof, wherein R¹ is selected from an optionally substituted aliphatic group having a mass between about 100 Da to about 500 Da, about 500 Da to about 1,000 Da, about 1,000 Da to about 2,000 Da, about 2,000 Da to about 5,000 Da, about 5,000 Da to about 10,000 Da, about 10,000 Da to about 20,000 Da, about 20,000 Da to about 50,000 Da. or about 50,000 Da to about 100,000 Da; and
(d) one or more binders.

In one aspect, the present disclosure provides pharmaceutical compositions, formulations, and unit dosage forms comprising:
(a) Compound 1:

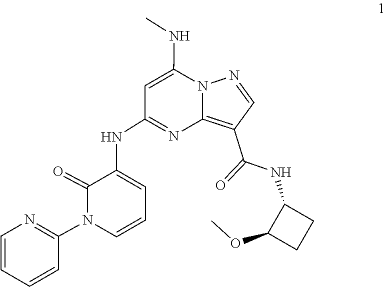 [SMILES: CNc1cc(Nc2cccn(-c3ccccn3)c2=O)nc2c(C(=O)N[C@@H]3CC[C@H]3OC)cnn12]

or a solvate thereof, or a pharmaceutically acceptable salt thereof;
(b) one or more fillers or diluents;
(c) one or more solubilizers of formula I-A:

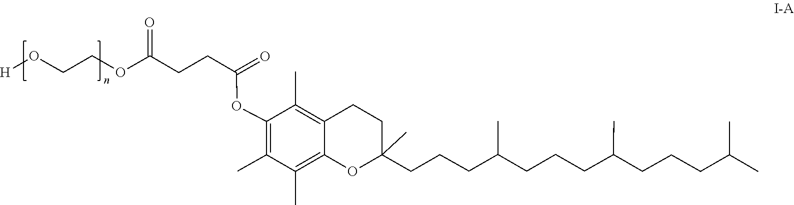

or a pharmaceutically acceptable salt thereof, wherein n is between about 10 to about 50, about 50 to about 100, about 100 to about 200, about 200 to about 500, about 500 to about 1,000, about 1,000 to about 2,000, about 2,000 to about 5,000, or about 5,000 to about 10,000. In some embodiments, n is about 1,000; and
(d) one or more binders.

In one aspect, the present disclosure provides pharmaceutical compositions, formulations, and unit dosage forms comprising:
(a) Compound 1:

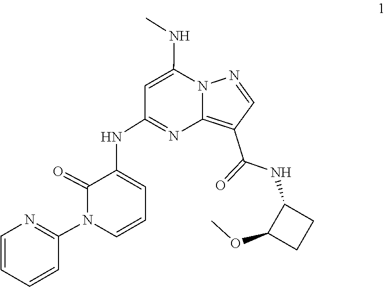 [SMILES: CNc1cc(Nc2cccn(-c3ccccn3)c2=O)nc2c(C(=O)N[C@@H]3CC[C@H]3OC)cnn12]

or a solvate thereof, or a pharmaceutically acceptable salt thereof;
(b) one or more fillers or diluents;
(c) Vitamin E TPGS; and
(d) one or more binders.

In one aspect, the present disclosure provides pharmaceutical compositions, formulations, and unit dosage forms comprising:
(a) Compound 1:

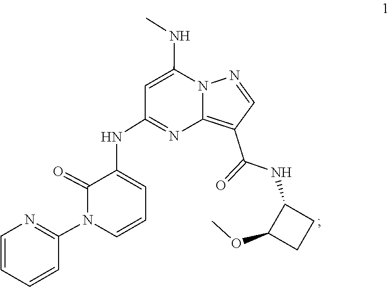 [SMILES: CNc1cc(Nc2cccn(-c3ccccn3)c2=O)nc2c(C(=O)N[C@@H]3CC[C@H]3OC)cnn12]

(b) one or more fillers or diluents;
(c) one or more solubilizers; and
(d) one or more binders.

In some embodiments, the diluent is selected from microcrystalline cellulose, sucrose, lactose, starch, magnesium stearate, sorbitol, xylitol, and mannitol.

In some embodiments, Compound 1 is in the form of a solvate, e.g., a hydrate, or a pharmaceutically acceptable salt of such solvate. In some embodiments, Compound 1 is in the form of a free base. In some embodiments, Compound 1 is in the form of a crystalline solid. In some embodiments, the crystalline solid is Form C.

In one aspect, the present disclosure provides pharmaceutical compositions, formulations, and unit dosage forms comprising:
(a) Compound 1:

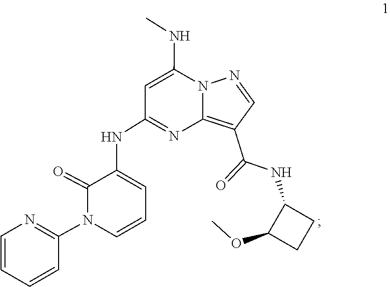 [SMILES: CNc1cc(Nc2cccn(-c3ccccn3)c2=O)nc2c(C(=O)N[C@@H]3CC[C@H]3OC)cnn12]

(b) one or more fillers or diluents;
(c) Vitamin E TPGS; and
(d) one or more binders.

In one aspect, the present disclosure provides pharmaceutical compositions, formulations, and unit dosage forms comprising:
(a) Compound 1:

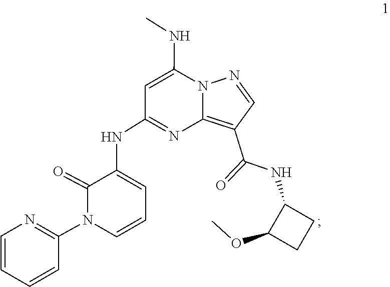 [SMILES: CNc1cc(Nc2cccn(-c3ccccn3)c2=O)nc2c(C(=O)N[C@@H]3CC[C@H]3OC)cnn12]

(b) microcrystalline cellulose;
(c) Vitamin E TPGS; and
(d) one or more binders.

In one aspect, the present disclosure provides pharmaceutical compositions, formulations, and unit dosage forms comprising:
(a) Compound 1:

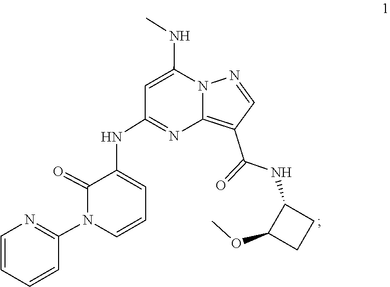 [SMILES: CNc1cc(Nc2cccn(-c3ccccn3)c2=O)nc2c(C(=O)N[C@@H]3CC[C@H]3OC)cnn12]

(b) Avicel PH101;
(c) Vitamin E TPGS; and
(d) one or more binders.

In one aspect, the present disclosure provides pharmaceutical compositions, formulations, and unit dosage forms comprising:
(a) Compound 1:

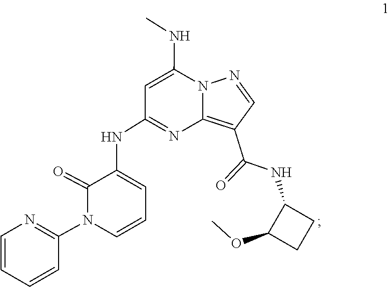 [SMILES: CNc1cc(Nc2cccn(-c3ccccn3)c2=O)nc2c(C(=O)N[C@@H]3CC[C@H]3OC)cnn12]

(b) microcrystalline cellulose;
(c) Vitamin E TPGS; and
(d) Povidone.

In one aspect, the present disclosure provides pharmaceutical compositions, formulations, and unit dosage forms comprising:
(a) Compound 1:

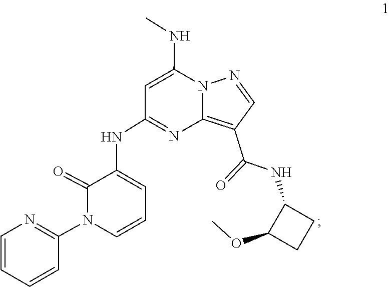 [SMILES: CNc1cc(Nc2cccn(-c3ccccn3)c2=O)nc2c(C(=O)N[C@@H]3CC[C@H]3OC)cnn12]

(b) Avicel PH101;
(c) Vitamin E TPGS; and
(d) PVP K30.

In one aspect, the present disclosure provides pharmaceutical compositions, formulations, and unit dosage forms consisting essentially of:
(a) Compound 1:

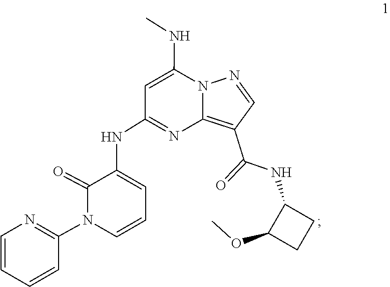 [SMILES: CNc1cc(Nc2cccn(-c3ccccn3)c2=O)nc2c(C(=O)N[C@@H]3CC[C@H]3OC)cnn12]

(b) Avicel PH101;
(c) Vitamin E TPGS; and
(d) PVP K30.

In one aspect, the present disclosure provides pharmaceutical compositions, formulations, and unit dosage forms consisting of:
(a) Compound 1:

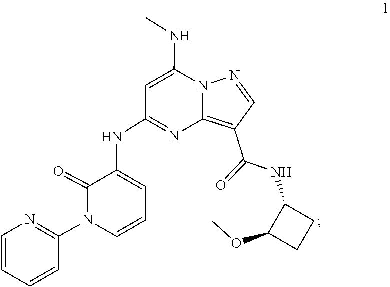 [SMILES: CNc1cc(Nc2cccn(-c3ccccn3)c2=O)nc2c(C(=O)N[C@@H]3CC[C@H]3OC)cnn12]

(b) Avicel PH101;
(c) Vitamin E TPGS; and
(d) PVP K30.

In some embodiments, the solubilizer is selected from a vitamin E conjugate. In some embodiments, the solubilizer can include one or more solubilizers of formula I:

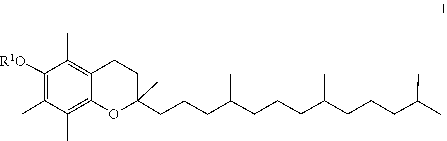

or a pharmaceutically acceptable salt thereof, wherein R¹ is selected from an optionally substituted aliphatic group having a mass between about 100 Da to about 500 Da, about 500 Da to about 1,000 Da, about 1,000 Da to about 2,000 Da, about 2,000 Da to about 5,000 Da, about 5,000 Da to about 10,000 Da, about 10,000 Da to about 20,000 Da, about 20,000 Da to about 50,000 Da, or about 50,000 Da to about 100,000 Da.

In some embodiments, the solubilizer can include one or more solubilizers of formula I-A:

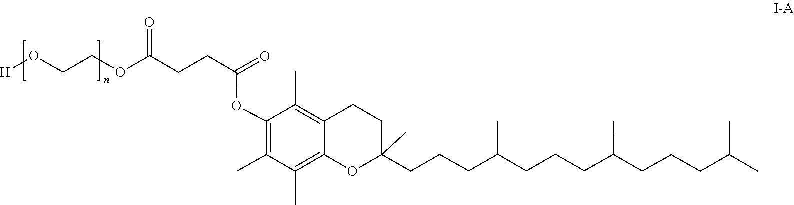

or a pharmaceutically acceptable salt thereof, wherein n is between about 10 to about 50, about 50 to about 100, about 100 to about 200, about 200 to about 500, about 500 to about 1,000, about 1,000 to about 2,000, about 2,000 to about 5,000, or about 5,000 to about 10,000. In some embodiments, n is between about 1 to about 5, about 5 to about 10, about 10 to about 20, about 20 to about 50, about 50 to about 100, about 100 to about 200, about 200 to about 500, or about 500 to about 1,000. In some embodiments, n is about 88. In some embodiments, n is about 100. In some embodiments, n is about 1,000. In some embodiments, the solubilizer can include Vitamin E polyethylene glycol succinate (Vitamin E TPGS).

In another aspect, pharmaceutical compositions, formulations, and unit dosage forms can include:
(a) Compound 1:

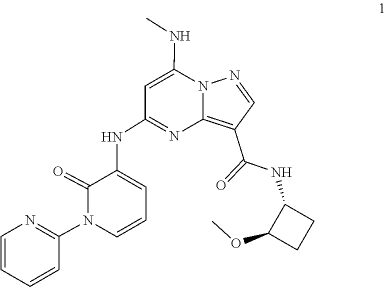 [SMILES: CNc1cc(Nc2cccn(-c3ccccn3)c2=O)nc2c(C(=O)N[C@@H]3CC[C@H]3OC)cnn12]

or a solvate thereof, or a pharmaceutically acceptable salt thereof;
   wherein Compound 1 is in the form of a spray-dried dispersion (SDD).

In some embodiments, the SDD can include a cellulosic polymer such as hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate, or carboxymethyl ethyl cellulose. In some embodiments, the SDD can include a cellulosic polymer that is at least partially ionized at physiologically relevant pHs, for example, hydroxypropyl methyl cellulose acetate succinate, hydroxypropyl methyl cellulose phthalate, cellulose acetate phthalate, cellulose acetate trimellitate, or carboxymethyl ethyl cellulose. In some embodiments, the SDD can include hydroxypropyl methyl cellulose acetate succinate (HPMCAS). In some embodiments, the SDD can include HPMC-AS-M (hydroxypropylmethylcellulose-acetate, succinate, Grade M).

The SDD can include a plurality of particles. The plurality of particles can be a powder or granules. The powder or granules can have an average particle size of less than 200 microns, less than 175 microns, less than 150 microns, less than 125 microns, less than 100 microns, less than 75 microns, less than 50 microns, less than 25 microns, less than 10 microns, or less than 5 microns. The powder or granules can have an average particle size of greater than 1 microns, greater than 3 microns, greater than 5 microns, greater than 10 microns, greater than 15 microns, greater than 20 microns, greater than 25 microns, greater than 30 microns, greater than 35 microns, or greater than 40 microns.

In some embodiments, the SDD can include Compound 1 or a solvate or a pharmaceutically acceptable salt thereof in a particular ratio to the cellulosic polymer, for example, about 10:1, 9:1, 8:1, 7:1, 6:1, 5:1, 4:1, 3:1, 2.5:1 2:1, 1.5:1, 1:1, 1:1.5, 1:2, 1:2.5, 1:3, 1:4, 1:5, 1:6, 1:7, 1:8, 1:9, or about 1:10 (as a w/w ratio). In some embodiments, the ratio is about 5:1 to about 1:5. In some embodiments, the ratio is about 10:1 to about 1:10, about 2.5:1 to about 1:2.5, or about 1.5:1 to about 1:1.5. In some embodiments, the ratio is about 1:1 (alternately expressed as 50:50).

In some embodiments, the SDD can be a spray-dried about 2:1 to about 1:2 (w/w) mixture of Compound 1 with HPMC-AS-M. In some embodiments, the SDD can be a spray-dried about 1:1 (w/w) mixture of Compound 1 with HPMC-AS-M.

In some embodiments, the pharmaceutical composition, formulation, or unit dosage form comprises:
a) about 1-4 mg of Compound 1 or a solvate or a pharmaceutically acceptable salt thereof, optionally as an SDD such as one of those described herein;
b) about 35-140 mg of a first filler such as mannitol;
c) optionally, about 35-140 mg of a second filler such as microcrystalline cellulose;
d) about 4-18 mg of a disintegrant such as croscarmellose sodium;
e) about 0.5-3.0 mg of a glidant such as colloidal silicon dioxide;
f) about 0.4-2.0 mg of a lubricant such as sodium stearyl fumarate; and
g) optionally, about 0.5-2 mg of a wetting agent such as sodium lauryl sulfate.

In some embodiments, each of b), c), d), and f) can be distributed between granules comprising a) and an extragranular portion of the pharmaceutical composition, formulation, or unit dosage form.

In some embodiments, the pharmaceutical composition, formulation, or unit dosage form comprises:
a) about 2.5-10 mg of Compound 1 or a solvate or a pharmaceutically acceptable salt thereof, optionally as an SDD such as one of those described herein;
b) about 30-130 mg of a first filler such as mannitol;
c) optionally, about 30-130 mg of a second filler such as microcrystalline cellulose;
d) about 4-20 mg of a disintegrant such as croscarmellose sodium;
e) about 0.5-4.0 mg of a glidant such as colloidal silicon dioxide;
f) optionally, about 0.5-4.0 mg of a wetting agent such as sodium lauryl sulfate; and
g) about 0.4-2.5 mg of a lubricant such as sodium stearyl fumarate.

In some embodiments, each of b), c), d), and g) can be distributed between granules comprising a) and an extragranular portion of the pharmaceutical composition, formulation, or unit dosage form.

In some embodiments, the pharmaceutical composition, formulation, or unit dosage form comprises:
a) about 7.5-30 mg of Compound 1 or a solvate or a pharmaceutically acceptable salt thereof, optionally as an SDD such as one of those described herein;
b) about 25-130 mg of a first filler such as mannitol;
c) optionally, about 25-130 mg of a second filler such as microcrystalline cellulose;
d) about 4-20 mg of a disintegrant such as croscarmellose sodium;
e) about 0.5-4.0 mg of a glidant such as colloidal silicon dioxide;
f) optionally, about 0.5-4.0 mg of a wetting agent such as sodium lauryl sulfate; and
g) about 0.4-4.0 mg of a lubricant such as sodium stearyl fumarate.

In some embodiments, each of b), c), d), and g) can be distributed between granules comprising a) and an extragranular portion of the pharmaceutical composition, formulation, or unit dosage form.

In some embodiments, the pharmaceutical composition, formulation, or unit dosage form comprises:
a) about 12.5-50 mg of Compound 1 or a solvate or a pharmaceutically acceptable salt thereof, optionally as an SDD such as one of those described herein;
b) about 20-95 mg of a first filler such as mannitol;
c) optionally, about 20-95 mg of a second filler such as microcrystalline cellulose;
d) about 4-20 mg of a disintegrant such as croscarmellose sodium;
e) about 0.5-4.0 mg of a glidant such as colloidal silicon dioxide;
f) optionally, about 0.5-4.0 mg of a wetting agent such as sodium lauryl sulfate; and
g) about 0.4-4.0 mg of a lubricant such as sodium stearyl fumarate.

In some embodiments, each of b), c), d), and g) can be distributed between granules comprising a) and an extragranular portion of the pharmaceutical composition, formulation, or unit dosage form.

In some embodiments, the pharmaceutical composition, formulation, or unit dosage form comprises:
a) about 25-75 mg of Compound 1 or a solvate or a pharmaceutically acceptable salt thereof, optionally as an SDD such as one of those described herein;
b) about 24-96 mg of a first filler such as mannitol;
c) optionally, about 24-96 mg of a second filler such as microcrystalline cellulose;
d) about 6-25 mg of a disintegrant such as croscarmellose sodium;
e) about 1.0-5.0 mg of a glidant such as colloidal silicon dioxide;
f) optionally, about 1.0-5.0 mg of a wetting agent such as sodium lauryl sulfate; and
g) about 0.7-2.6 mg of a lubricant such as sodium stearyl fumarate.

In some embodiments, each of b), c), d), and g) can be distributed between granules comprising a) and an extragranular portion of the pharmaceutical composition, formulation, or unit dosage form.

In some embodiments, the pharmaceutical composition, formulation, or unit dosage form comprises one of the following amounts or % w/w of ingredients shown below.

| | 2 mg | 5 mg | | 15 mg |
|---|---|---|---|---|
| Ingredients | w/w % | w/w % | w/w % | w/w % |
| Spray Dried | 1-5 | 2.5-10.0 | 2.5-10.0 | 7.0-40.0 |
| Intermediate | | | | |
| Microcrystalline | 0-80 or 18.0- | 0-75.0 or 18.0- | 0-75.0 or 18.0- | 0-70.0 or 18.0- |
| Cellulose | 80.0 | 75.0 | 75.0 | 70.0 |
| Mannitol | 18.0-80.0 | 18.0-75.0 | 18.0-75.0 | 18.0-70.0 |
| Croscarmellose | 4.5-26.0 | 5.0-18.0 | 6.5-27.0 | 4.6-20.0 |
| Sodium | | | | |
| Colloidal Silicon | 0.25-2.0 | 0.25-2.0 | 0.25-2.0 | 0.5-2.2 |
| Dioxide | | | | |
| Sodium Lauryl | 0.0-2 | 0.5-2 | 0.0-2.0 | 0.0-2.0 |
| Sulfate | | | | |
| Sodium Stearyl | 0.2-1.5 | 0.1-0.5 | 0.25-1.0 | 0.1-0.5 |
| Fumarate | | | | |

The Spray Dried Intermediate can include a spray-dried solid of Compound 1 and a cellulosic polymer, such as a 1.5:1 to 1:1.5 w/w ratio of Compound 1: HPMC-AS (hydroxypropylmethylcellulose-acetate, succinate).

| | 25 mg | | 50 mg |
|---|---|---|---|
| Ingredients | w/w % | w/w % | w/w % |
| Spray Dried | 23.0-52.0 | 16.0-66.0 | 20.0-80.0 |
| intermediate | | | |
| Microcrystalline | 0-70.0 or | 0-60.0 or | 0-58.0 or |
| Cellulose | 17.0-70.0 | 16.5-60.0 | 12.5-58.0 |
| Mannitol | 12.5-51.0 | 12.0-48.0 | 8.0-38.0 |
| Croscarmellose | 4.9-20.0 | 4.0-16.0 | 4.9-20.0 |
| Sodium | | | |
| Colloidal Silicon | 0.5-2.0 | 0.5-2.0 | 0.5-2.0 |
| Dioxide | | | |
| Sodium Lauryl | 0.5-2.0 | 0.0-2.0 | 0.5-2.0 |
| Sulfate | | | |
| Sodium Stearyl | 0.3-1.5 | 0.3-1.5 | 0.3-1.5 |
| Fumarate | | | |

The Spray Dried Intermediate can include a spray-dried solid of Compound 1 and a cellulosic polymer, such as a 1.5:1 to 1:1.5 w/w ratio of Compound 1: HPMC-AS (hydroxypropylmethylcellulose-acetate, succinate).

In some embodiments, the pharmaceutical composition, formulation, or unit dosage form comprises one of the following amounts or % w/w of ingredients shown below.

| | 2 mg | | 5 mg | | | | 15 mg | |
|---|---|---|---|---|---|---|---|---|
| Ingredients | mg | w/w % | mg | w/w % | mg | w/w % | mg | w/w % |
| Spray Dried | 4.0 | 2.2 | 10 | 5.6 | 10 | 5.6 | 30.0 | 17.9 |
| Intermediate | | | | | | | | |
| Microcrystalline | 75.5 | 47.5 | 81.7 | 45.4 | 72.5 | 45.8 | 61.5 | 36.6 |
| Cellulose | | | | | | | | |
| Mannitol | 69.3 | 38.5 | 65.6 | 36.4 | 66.3 | 36.8 | 56.5 | 33.6 |
| Croscarmellose | 18.0 | 10.0 | 17.7 | 9.8 | 18.0 | 10.0 | 17.0 | 10.2 |
| Sodium | | | | | | | | |
| Colloidal Silicon | 1.8 | 1.0 | 1.8 | 1.0 | 1.8 | 1.0 | 1.8 | 1.1 |
| Dioxide | | | | | | | | |
| Sodium Lauryl | 0.0 | 0.0 | 1.8 | 1.0 | 0.0 | 0.0 | 0.0 | 0.0 |
| Sulfate | | | | | | | | |
| Sodium Stearyl | 1.4 | 0.75 | 1.4 | 0.75 | 1.4 | 0.75 | 1.4 | 0.83 |
| Fumarate | | | | | | | | |
| Total Weight or % | 180.0 | 100.0 | 180.1 | 100.0 | 180.0 | 100.0 | 168.0 | 100.0 |

The Spray Dried Intermediate can include a spray-dried solid of Compound 1 and a cellulosic polymer, such as a 1.5:1 to 1:1.5 w/w ratio of Compound 1: HPMC-AS (hydroxypropylmethylcellulose-acetate, succinate).

| | 25 mg | | | | 50 mg | |
|---|---|---|---|---|---|---|
| Ingredients | mg | w/w % | mg | w/w % | mg | w/w % |
| Spray Dried | 50.0 | 27.8 | 50.0 | 33.3 | 100 | 40 |
| intermediate | | | | | | |
| Microcrystalline | 61.8 | 34.3 | 49.4 | 33.0 | 70.5 | 28.2 |
| Cellulose | | | | | | |
| Mannitol | 45.6 | 25.3 | 35.9 | 24.0 | 48 | 19.2 |
| Croscarmellose | 17.7 | 9.8 | 12.0 | 8.0 | 24.6 | 9.8 |
| Sodium | | | | | | |
| Colloidal Silicon | 1.8 | 1.0 | 1.5 | 1.0 | 2.5 | 1 |
| Dioxide | | | | | | |
| Sodium Lauryl Sulfate | 1.8 | 1.0 | 0.0 | 0.0 | 2.5 | 1 |
| Sodium Stearyl | 1.4 | 0.75 | 1.2 | 0.75 | 1.9 | 0.75 |
| Fumarate | | | | | | |
| Total Weight or % | 180.1 | 100.0 | 150.0 | 100.0 | 250.0 | 100.0 |

The Spray Dried Intermediate can include a spray-dried solid of Compound 1 and a cellulosic polymer, such as a 1.5:1 to 1:1.5 w/w ratio of Compound 1: HPMC-AS (hydroxypropylmethylcellulose-acetate, succinate).

In another aspect, a method of manufacturing a formulation described above, can include the steps of:
(1) providing an SDD comprising Compound 1 or a solvate or a pharmaceutically acceptable salt thereof and a cellulosic polymer such as HPMC-AS;
(2) blending the SDD with intragranular excipients;
(3) delumping the mixture;
(4) blending the mixture;
(5) dry granulating the mixture, e.g., with a roller compactor against a 1.00 mm screen, to produce a dry granulate;
(6) blending the dry granulate with extragranular excipients; and
(7) optionally, filling the formulation into an appropriate unit container such as capsules. Different amounts of intra- and extra-granular excipients may be used to produce the blends that comprise capsules such as 2 mg, 5 mg, 15 mg, 25 mg, and 50 mg strength capsules such that the 2 mg, 5 mg, 15 mg, and 25 mg blends can fit into size #3 HPMC capsules and the 50 mg blend into size #2 HPMC capsules.

In some embodiments, the SDD is prepared as follows:
(1) providing a solution of Compound 1 or a pharmaceutically acceptable salt thereof and HPMC-AS;
(2) filtering the solution, e.g., through a 100-mesh screen;
(3) spray drying the solution to produce an SDI (spray dried intermediate);
(4) optionally, drying the SDI, e.g., by secondary tray drying;
   thus providing the SDD.

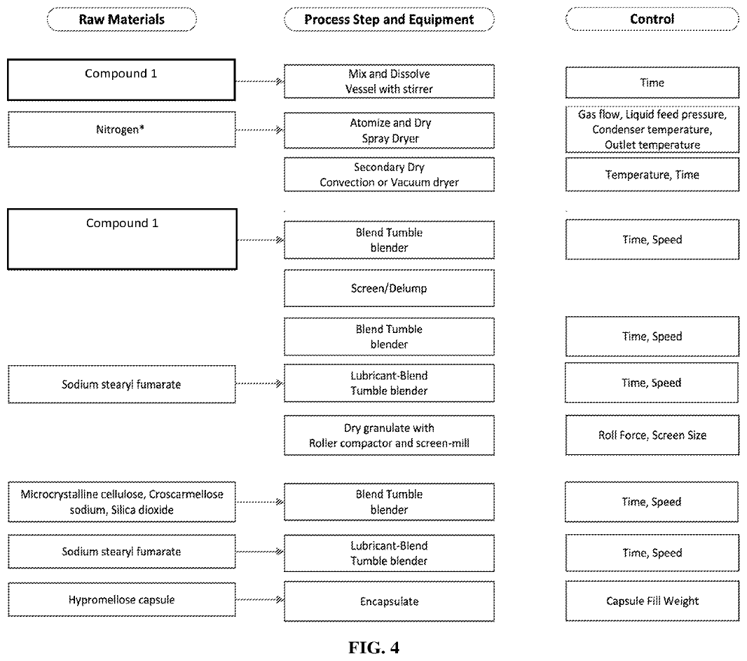

In some embodiments, the manufacturing process is substantially as described in the flow diagram shown in FIG. 4.

In some embodiments, the binder can include one or more binders of formula II:

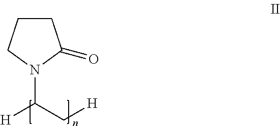

or a pharmaceutically acceptable salt thereof, wherein n is between about 10 to about 50, about 50 to about 100, about 100 to about 200, about 200 to about 500, about 500 to about 1,000, about 1,000 to about 2,000, about 2,000 to about 5,000, or about 5,000 to about 10,000. In some embodiments, n is about 1,000. In some embodiments, the binder can include a molecular weight between about 10,000 Da and about 25,000 Da, about 25,000 and about 50,000 Da, about 50,000 Da and about 60,000 Da, or about 60,000 Da and about 70,000 Da. In some embodiments, the binder can include povidone. In some embodiments, the binder can include PVP30.

In some embodiments, the diluent can include a cellulose. In some embodiments, the cellulose is microcrystalline cellulose. In some embodiments, the diluent can include Avicel® PH-101. In some embodiment the diluent can have a nominal particle size between about 10 mm to about 20 mm, about 20 mm to about 30 mm, about 30 mm to about 40 mm, about 40 mm to about 50 mm, about 50 mm to about 60 mm, about 60 mm to about 70 mm, or about 70 mm to about 80 mm. In some embodiments, the diluent can have a nominal particle size of about 50 mm. In some embodiments, the diluent can have a moisture content between about 0.5% w/w to about 1% w/w, about 1% w/w to about 1.5% w/w, about 1.5% w/w to about 2% w/w, about 2% w/w to about 2.5% w/w, about 2.5% w/w to about 3% w/w, about 3% w/w to about 3.5% w/w, about 3.5% w/w to about 4% w/w, about 4% w/w to about 4.5% w/w, or about 4.5% w/w to about 5% w/w. In some embodiments, the diluent can have a moisture content between about 3% w/w to about 5% w/w. In some embodiments, the diluent can have a loose bulk density between about 0.1 g/cm³ to about 0.15 g/cm³, about 0.15 g/cm³ to about 0.2 g/cm³, about 0.2 g/cm³ to about 0.25 g/cm³, about 0.25 g/cm³ to about 0.3 g/cm³, about 0.3 g/cm³ to about 0.35 g/cm³, about 0.35 g/cm³ to about 0.4 g/cm³, about 0.4 g/cm³ to about 0.45 g/cm³, or about 0.45 g/cm³ to about 0.5 g/cm³. In some embodiments, the diluent can have a loose bulk density between about 0.26 g/cm³ to about 0.31 g/cm³.

In some embodiments, the microcrystalline cellulose can have an average molecular weight between about 10,000 Da and about 25,000 Da, about 25,000 Da and about 50,000 Da, about 50,000 Da and about 60,000 Da, or about 60,000 Da and about 80,000 Da.

### Capsule Unit Dosage Forms

In certain embodiments, the unit dosage form can include a capsule or tablet. Capsules, tablets, and other unit dosage forms are described in additional detail infra. As noted above, pharmaceutical composition, formulations, and unit dosage forms may comprise:
(a) Compound 1, or a solvate thereof, or a pharmaceutically acceptable salt thereof;
(b) one or more fillers or diluents;
(c) one or more solubilizers;
(d) one or more binders;
(e) optionally, one or more glidants;
(f) optionally, one or more lubricants; and
(g) optionally, one or more surfactants.

In some embodiments, the unit dosage form can include (a), (b), (c), (d), optionally (e), optionally (f), and optionally (g) as described supra. In some embodiments, the unit dosage form comprises (a), (b), (c), and (d). In some embodiments, the unit dosage form consists essentially of (a), (b), (c), and (d). In some embodiments, the unit dosage form consists of (a), (b), (c), and (d).

In some embodiments, the unit dosage form can include an amount of Compound 1 or a solvate or pharmaceutically acceptable salt thereof between about 1 mg to about 2.5 mg, about 2.5 mg to about 5 mg, about 5 mg to about 7.5 mg, about 7.5 mg to about 10 mg, about 10 mg to about 12.5 mg, about 12.5 mg to about 15 mg, about 15 mg to about 17.5 mg, about 17.5 mg to about 20 mg, about 20 mg to about 22.5 mg, about 22.5 mg to about 25 mg, about 25 mg to about 27.5 mg, about 27.5 mg to about 30 mg, about 30 mg to about 32.5 mg, about 32.5 mg to about 35 mg, about 35 mg to about 37.5 mg, about 37.5 mg to about 40 mg, about 40 mg to about 45 mg, or about 45 mg to about 50 mg. In some embodiments, the unit dosage form can include an amount of about 5 mg. In some embodiments, the unit dosage form can include an amount of about 25 mg.

In some embodiments, the unit dosage form can include an amount of Compound 1 or a solvate or pharmaceutically acceptable salt thereof between about 1% w/w to about 2.5% w/w, about 2.5% w/w to about 5% w/w, about 5% w/w to about 7.5% w/w, about 7.5% w/w to about 10% w/w, about 10% w/w to about 12.5% w/w, about 12.5% w/w to about 15% w/w, about 15% w/w to about 17.5% w/w, or about 17.5% w/w to about 20% w/w. In some embodiments, the unit dosage form can include an amount of about 10% w/w.

In some embodiments, the unit dosage form can include an amount of (b) between about 15 mg to about 17.5 mg, about 17.5 mg to about 20 mg, about 20 mg to about 22.5 mg, about 22.5 mg to about 25 mg, about 25 mg to about 27.5 mg, about 27.5 mg to about 30 mg, about 30 mg to about 32.5 mg, about 32.5 mg to about 35 mg, about 35 mg to about 37.5 mg, about 37.5 mg to about 40 mg, about 40 mg to about 45 mg, about 45 mg to about 50 mg, about 50 mg to about 75 mg, about 75 mg to about 100 mg, about 100 mg to about 125 mg, about 125 mg to about 150 mg, about 150 mg to about 175 mg, about 175 mg to about 200 mg, or about 200 mg to about 225 mg. In some embodiments, the unit dosage form can include an amount of (b) of about 34 mg. In some embodiments, the unit dosage form can include an amount of (b) of about 170 mg.

In some embodiments, the unit dosage form can include an amount of (b) between about 20% w/w to about 30% w/w, about 30% w/w to about 40% w/w, about 40% w/w to about 50% w/w, about 50% w/w to about 60% w/w, about 60% w/w to about 70% w/w, about 70% w/w to about 80% w/w, or about 80% w/w to about 90% w/w. In some embodiments, the unit dosage form comprises an amount of (b) of about 68% w/w.

In some embodiments, the unit dosage form comprises an amount of (c) between about 1 mg to about 2.5 mg, about 2.5 mg to about 5 mg, about 5 mg to about 7.5 mg, about 7.5 mg to about 10 mg, about 10 mg to about 12.5 mg, about 12.5 mg to about 15 mg, about 15 mg to about 17.5 mg, about 17.5 mg to about 20 mg, about 20 mg to about 22.5 mg, about 22.5 mg to about 25 mg, about 25 mg to about 27.5 mg, about 27.5 mg to about 30 mg, about 30 mg to about 32.5 mg, about 32.5 mg to about 35 mg, about 35 mg to about 37.5 mg, about 37.5 mg to about 40 mg, about 40 mg to about 45 mg, about 45 mg to about 50 mg, about 50 mg to about 75 mg, about 75 mg to about 100 mg, about 100 mg to about 125 mg, or about 125 mg to about 150 mg. In some embodiments, the unit dosage form comprises an amount of (c) of about 10 mg. In some embodiments, the unit dosage form comprises an amount of (c) of about 50 mg.

In some embodiments, the unit dosage form comprises an amount of (c) between about 2% w/w to about 5% w/w, about 5% w/w to about 10% w/w, about 10% w/w to about 15% w/w, about 15% w/w to about 20% w/w, about 20% w/w to about 25% w/w, about 25% w/w to about 30% w/w, about 30% w/w to about 35% w/w, or about 35% w/w to about 40% w/w. In some embodiments, the unit dosage form comprises an amount of (c) of about 20% w/w.

In some embodiments, the unit dosage form comprises an amount of (d) between about 0.25 mg to about 0.5 mg, about 0.5 mg to about 0.75 mg, about 0.75 mg to about 1 mg, about 1 mg to about 2 mg, about 2 mg to about 3 mg, about 3 mg to about 4 mg, about 4 mg to about 5 mg, about 5 mg to about 6 mg, about 6 mg to about 7 mg, about 7 mg to about 8 mg, about 8 mg to about 9 mg, or about 9 mg to about 10 mg.

In some embodiments, the unit dosage form comprises an amount of (d) between about 0.25% w/w to about 0.5% w/w, about 0.5% w/w to about 0.75% w/w, about 0.75% w/w to about 1% w/w, about 1% w/w to about 1.25% w/w, about 1.25% w/w to about 1.5% w/w, about 1.5% w/w to about 1.75% w/w, about 1.75% w/w to about 2% w/w, about 2% w/w to about 2.5% w/w, about 2.5% w/w to about 3% w/w, about 3% w/w to about 3.5% w/w, or about 3.5% w/w to about 4% w/w. In some embodiments, the unit dosage form comprises an amount of (d) of about 2% w/w.

In some embodiments, the unit dosage form comprises amounts of (a), (b), (c), and (d) of:
(a) between about 2.5 mg to about 7.5 mg;
(b) between about 17 mg to about 51 mg;
(c) between about 5 mg to about 15 mg; and
(d) between about 0.5 mg to about 1.5 mg.

In some embodiments, the unit dosage form comprises amounts of (a), (b), (c), and (d) of:
(a) about 5 mg;
(b) about 34 mg;
(c) about 10 mg; and
(d) about 1 mg.

In some embodiments, the unit dosage form comprises amounts of (a), (b), (c), and (d) of:
(a) between about 12.5 mg to about 37.5 mg;
(b) between about 85 mg to about 255 mg;
(c) between about 25 mg to about 75 mg; and
(d) between about 2.5 mg to about 7.5 mg.

In some embodiments, the unit dosage form comprises amounts of (a), (b), (c), and (d) of:
(a) about 25 mg;
(b) about 170 mg;
(c) about 50 mg; and
(d) about 5 mg.

In some embodiments, the pharmaceutical composition, formulation, or unit dosage form of any embodiment herein can include granules, wherein the pharmaceutical composition, formulation, or unit dosage form can include intragranular and extragranular components as follows:
a) about 4 mg of an intragranular spray-dried 50:50 (w/w) mixture of Compound 1 with HPMC-AS-M (hydroxypropylmethylcellulose-acetate, succinate, Grade M);
b) about 69.3 mg intragranular mannitol;
c) about 69.3 mg intragranular microcrystalline cellulose;
d) about 9 mg intragranular croscarmellose sodium;
e) about 1.8 mg intragranular colloidal silicon dioxide;
f) about 0.9 mg intragranular sodium stearyl fumarate;
g) about 16.2 mg extragranular microcrystalline cellulose;
h) about 9 mg extragranular croscarmellose sodium; and
i) about 0.5 mg extragranular sodium stearyl fumarate.

In some embodiments, the pharmaceutical composition, formulation, or unit dosage form of any embodiment herein can include granules, wherein the pharmaceutical composition, formulation, or unit dosage form can include intragranular and extragranular components as follows:
a) about 10 mg of an intragranular spray-dried 50:50 (w/w) mixture of Compound 1 with HPMC-AS-M (hydroxypropylmethylcellulose-acetate, succinate, Grade M);
b) about 65.6 mg intragranular mannitol;
c) about 65.6 mg intragranular microcrystalline cellulose;
d) about 9 mg intragranular croscarmellose sodium;
e) about 1.8 mg intragranular colloidal silicon dioxide;
f) about 1.8 mg intragranular sodium lauryl sulfate;
g) about 0.9 mg intragranular sodium stearyl fumarate;
h) about 16.2 mg extragranular microcrystalline cellulose;
i) about 8.7 mg extragranular croscarmellose sodium; and
j) about 0.5 mg extragranular sodium stearyl fumarate.

In some embodiments, the pharmaceutical composition, formulation, or unit dosage form of any embodiment herein can include granules, wherein the pharmaceutical composition, formulation, or unit dosage form can include intragranular and extragranular components as follows:
a) about 30 mg of an intragranular spray-dried 50:50 (w/w) mixture of Compound 1 with HPMC-AS-M (hydroxypropylmethylcellulose-acetate, succinate, Grade M);
b) about 56.46 mg intragranular mannitol;
c) about 56.46 mg intragranular microcrystalline cellulose;
d) about 9 mg intragranular croscarmellose sodium;
e) about 1.8 mg intragranular colloidal silicon dioxide;
f) about 0.9 mg intragranular sodium stearyl fumarate;
g) about 5 mg extragranular microcrystalline cellulose;
h) about 8 mg extragranular croscarmellose sodium; and
i) about 0.38 mg extragranular sodium stearyl fumarate.

In some embodiments, the pharmaceutical composition, formulation, or unit dosage form of any embodiment herein can include granules, wherein the pharmaceutical composition, formulation, or unit dosage form can include intragranular and extragranular components as follows:
a) about 50 mg of an intragranular spray-dried 50:50 (w/w) mixture of Compound 1 with HPMC-AS-M (hydroxypropylmethylcellulose-acetate, succinate, Grade M);
b) about 45.6 mg intragranular mannitol;
c) about 45.6 mg intragranular microcrystalline cellulose;
d) about 9 mg intragranular croscarmellose sodium;
e) about 1.8 mg intragranular colloidal silicon dioxide;
f) about 1.8 mg intragranular sodium lauryl sulfate;
g) about 0.9 mg intragranular sodium stearyl fumarate;
h) about 16.2 mg extragranular microcrystalline cellulose;
i) about 8.7 mg extragranular croscarmellose sodium; and
j) about 0.5 mg extragranular sodium stearyl fumarate.

In some embodiments, the pharmaceutical composition, formulation, or unit dosage form of any embodiment herein can include granules, wherein the pharmaceutical composition, formulation, or unit dosage form can include intragranular and extragranular components as follows:
a) about 100 mg of an intragranular spray-dried 50:50 (w/w) mixture of Compound 1 with HPMC-AS-M (hydroxypropylmethylcellulose-acetate, succinate, Grade M);
b) about 48 mg intragranular mannitol;
c) about 48 mg intragranular microcrystalline cellulose;
d) about 12.5 mg intragranular croscarmellose sodium;
e) about 2.5 mg intragranular colloidal silicon dioxide;
f) about 2.5 mg intragranular sodium lauryl sulfate;
g) about 1.3 mg intragranular sodium stearyl fumarate;
h) about 22.5 mg extragranular microcrystalline cellulose;
i) about 12.1 mg extragranular croscarmellose sodium; and
j) about 0.6 mg extragranular sodium stearyl fumarate.

In some embodiments, Compound 1 can be in the form of a solvate, e.g., a hydrate, or a pharmaceutically acceptable salt of such solvate. In some embodiments, Compound 1 can be in the form of a free base. In some embodiments, Compound 1 can be in the form of a crystalline solid. In some embodiments, the crystalline solid can be Form C. In some embodiments, the crystalline solid can be Form A.

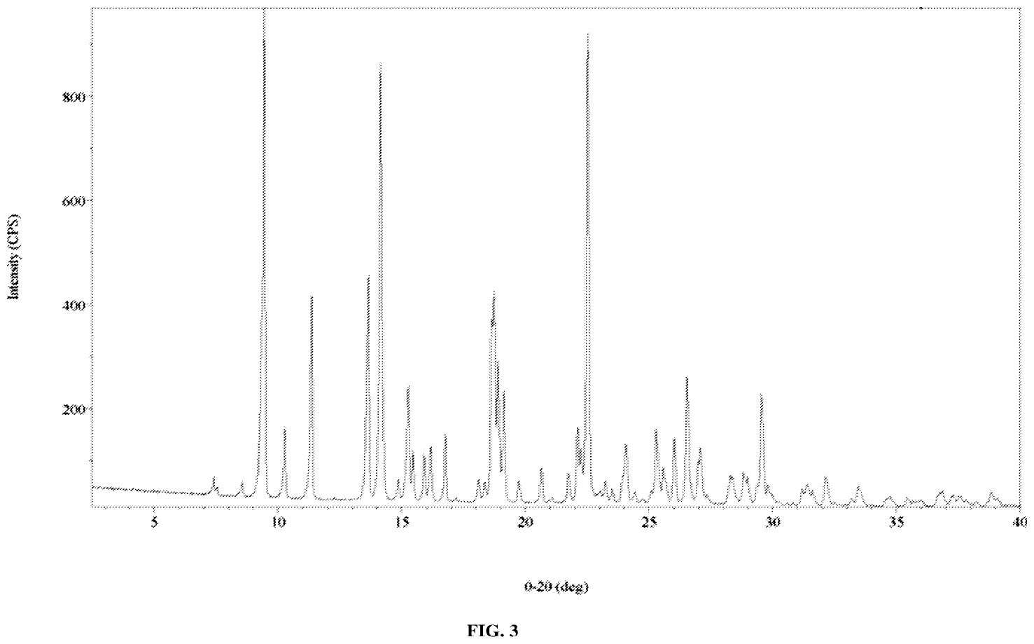

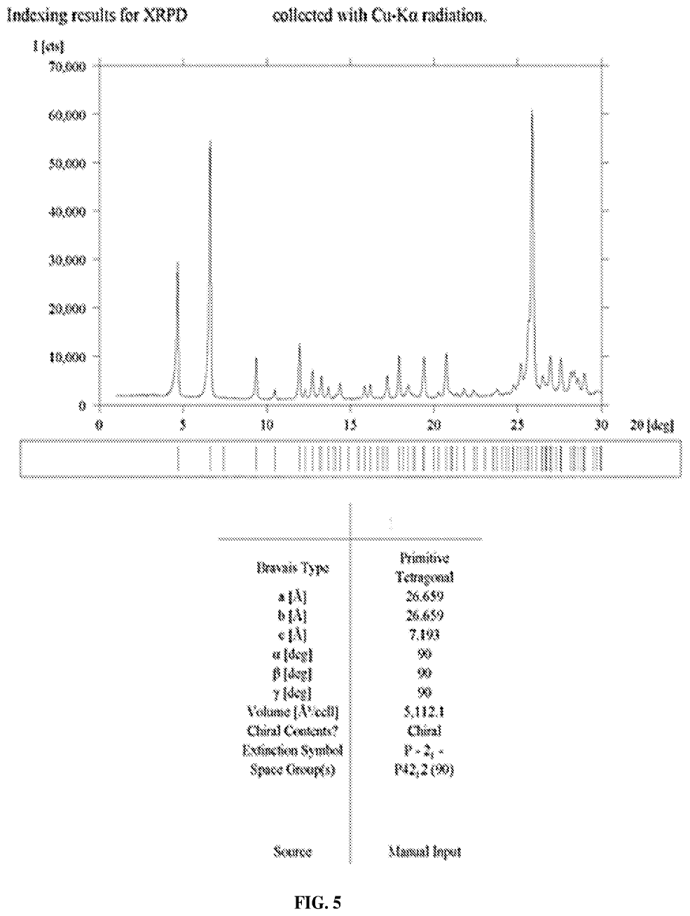

In certain embodiments, Compound 1 can be a crystalline compound of polymorphic Form C, characterized in that it exhibits an X-ray powder diffraction (XRPD) pattern comprising peaks substantially as shown in FIG. 3. In certain embodiments, Compound 1 can be a crystalline compound of polymorphic Form A, characterized in that it exhibits an X-ray powder diffraction (XRPD) pattern comprising peaks substantially as shown in FIG. 5. Any alternative solid form may be used in the formulations described herein; for example, amorphous forms, crystalline forms other than Form C or A, or mixtures thereof.

In certain embodiments, Compound 1 amorphous. In some embodiments, Compound 1 can be as a hydrate. In some embodiments, Compound 1 can be as a monohydrate.

### Methods of Formulation Production

In one aspect, the present disclosure provides a method of producing a pharmaceutical composition, formulation, or unit dosage form, the method comprising wet granulation of a mixture comprising Compound 1:

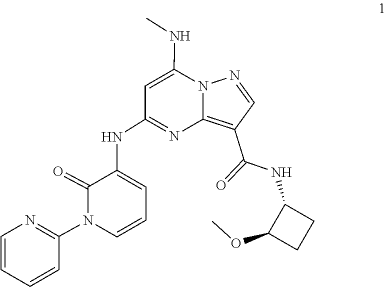 [SMILES: CNc1cc(Nc2cccn(-c3ccccn3)c2=O)nc2c(C(=O)N[C@@H]3CC[C@H]3OC)cnn12]

or a solvate thereof, or a pharmaceutically acceptable salt thereof; and one or more solubilizers.

In some embodiments, the method can include:
(1) wet granulation of the mixture; and
(2) drying the mixture.

In some embodiments, the method can include:
(1) wet granulation of the mixture;
(2) drying the mixture; and
(3) dry milling the mixture.

In some embodiments, the method can include:
(1) wet granulation of the mixture;
(2) drying the mixture;
(3) dry milling the mixture; and
(4) blending the mixture.

In some embodiments, the method can include:
(1) wet granulation of the mixture;
(2) drying the mixture;
(3) dry milling the mixture;
(4) blending the mixture; and
(5) encapsulating the mixture.

In some embodiments, the mixture can be de-lumped prior to wet granulation.

In some embodiments, the mixture can include:
(a) Compound 1;
(b) one or more diluents;
(c) one or more solubilizers;
(d) one or more binders;
(e) optionally, one or more glidants;
(f) optionally, one or more lubricants; and
(g) optionally, one or more surfactants.

In some embodiments, the mixture can include:
(a) Compound 1;
(b) one or more diluents;
(c) one or more solubilizers; and
(d) one or more binders.

In some embodiments, the mixture can include:
(a) Compound 1;
(b) one or more diluents;
(c) one or more solubilizers of formula I:

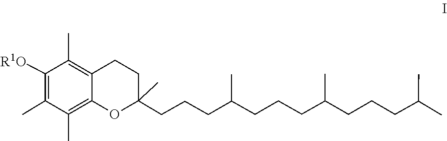

or a pharmaceutically acceptable salt thereof, wherein R¹ is selected from an optionally substituted aliphatic group having a mass between about 100 Da to about 500 Da, about 500 Da to about 1,000 Da, about 1,000 Da to about 2,000 Da, about 2,000 Da to about 5,000 Da, about 5,000 Da to about 10,000 Da, about 10,000 Da to about 20,000 Da, about 20,000 Da to about 50,000 Da, or about 50,000 Da to about 100,000 Da; and
(d) one or more binders.

In some embodiments, the mixture can include:
(a) Compound 1;
(b) one or more diluents;
(c) one or more solubilizers of formula I-A:

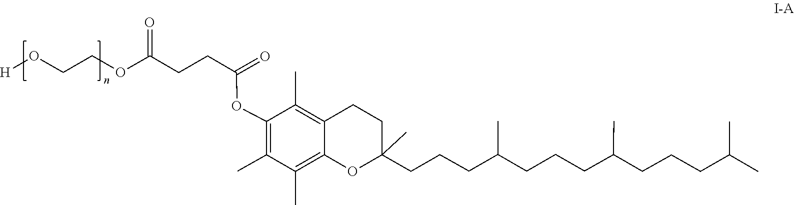

or a pharmaceutically acceptable salt thereof, wherein n is between about 10 to about 50, about 50 to about 100, about 100 to about 200, about 200 to about 500, about 500 to about 1,000, about 1,000 to about 2,000, about 2,000 to about 5,000, or about 5,000 to about 10,000; and
(d) one or more binders.

In some embodiments, n is between about 1 to about 5, about 5 to about 10, about 10 to about 20, about 20 to about 50, about 50 to about 100, about 100 to about 200, about 200 to about 500, or about 500 to about 1,000. In some embodiments, n is about 88. In some embodiments, n is about 100. In some embodiments, n is about 1,000.

In some embodiments, the mixture can include:
(a) Compound 1;
(b) one or more diluents;
(c) Vitamin E TPGS; and
(d) one or more binders.

In some embodiments, the mixture comprises:
(a) Compound 1;
(b) microcrystalline cellulose;
(c) Vitamin E TPGS; and
(d) one or more binders.

In some embodiments, the mixture can include:
(a) Compound 1;
(b) one or more diluents;
(c) one or more solubilizers; and
(d) one or more binders.

In some embodiments, the pharmaceutical composition, formulation, or unit dosage form can include:
a) about 5% about 15% (w/w) of Compound 1;
b) about 32% to about 100% (w/w) of a diluent;
c) about 10% about 30% (w/w) of a solubilizer; and
d) about 1% to about 3% (w/w) of a binder.

In some embodiments, the pharmaceutical composition, formulation, or unit dosage form can include:
a) about 10% (w/w) of Compound 1;
b) about 68% (w/w) of a diluent;
c) about 20% (w/w) of a solubilizer; and
d) about 2% (w/w) of a binder.

In some embodiments, the pharmaceutical composition, formulation, or unit dosage form can include:
a) about 20% (w/w) of Compound 1;
b) about 58% (w/w) of a diluent;
c) about 20% (w/w) of a solubilizer; and
d) about 2% (w/w) of a binder.

In some embodiments, the pharmaceutical composition, formulation, or unit dosage form can include:
a) about 30% (w/w) of Compound 1;
b) about 48% (w/w) of a diluent;
c) about 20% (w/w) of a solubilizer; and
d) about 2% (w/w) of a binder.

In some embodiments, the pharmaceutical composition, formulation, or unit dosage form can include:
a) about 40% (w/w) of Compound 1;
b) about 38% (w/w) of a diluent;
c) about 20% (w/w) of a solubilizer; and
d) about 2% (w/w) of a binder.

In some embodiments, the diluent can be microcrystalline cellulose, the solubilizer can be Vitamin G TPGS, and the binder can be povidone.

In some embodiments, the mixture can include:
(a) Compound 1;
(b) Avicel PH101;
(c) Vitamin E TPGS; and
(d) one or more binders.

In some embodiments, the mixture can include:
(a) Compound 1;
(b) microcrystalline cellulose;
(c) Vitamin E TPGS; and
(d) Povidone.

In some embodiments, the mixture can include:
(a) Compound 1;
(b) Avicel PH101;
(c) Vitamin E TPGS; and
(d) PVP K30.

In some embodiments, the mixture consists essentially of:
(a) Compound 1;
(b) Avicel PH101;
(c) Vitamin E TPGS; and
(d) PVP K30.

In some embodiments, the mixture consists of:
(a) Compound 1;
(b) Avicel PH101;
(c) Vitamin E TPGS; and
(d) PVP K30.

In some embodiments, the solubilizer can be selected from a vitamin E conjugate. In some embodiments, the solubilizer can include one or more solubilizers of formula I:

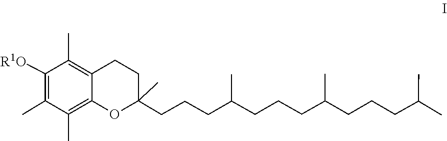

or a pharmaceutically acceptable salt thereof, wherein R¹ is selected from an optionally substituted aliphatic group having a mass between about 100 Da to about 500 Da, about 500 Da to about 1,000 Da, about 1,000 Da to about 2,000 Da, about 2,000 Da to about 5,000 Da, about 5,000 Da to about 10,000 Da, about 10,000 Da to about 20,000 Da, about 20,000 Da to about 50,000 Da, or about 50,000 Da to about 100,000 Da.

In some embodiments, the solubilizer can include one or more solubilizers of formula I-A:

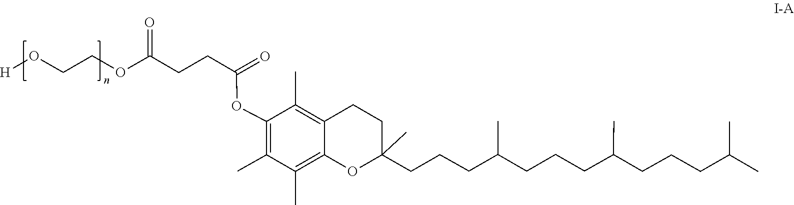

or a pharmaceutically acceptable salt thereof, wherein n is between about 10 to about 50, about 50 to about 100, about 100 to about 200, about 200 to about 500, about 500 to about 1,000, about 1,000 to about 2,000, about 2,000 to about 5,000, or about 5,000 to about 10,000. In some embodiments, n is about 1,000. In some embodiments, the solubilizer can include Vitamin E polyethylene glycol succinate (Vitamin E TPGS).

In some embodiments, the binder can include one or more binders of formula II:

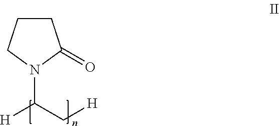

or a pharmaceutically acceptable salt thereof, wherein n is between about 10 to about 50, about 50 to about 100, about 100 to about 200, about 200 to about 500, about 500 to about 1,000, about 1,000 to about 2,000, about 2,000 to about 5,000, or about 5,000 to about 10,000. In some embodiments, n is about 1,000. In some embodiments, the binder can include povidone. In some embodiments, the binder can include PVP30.

In some embodiments, the diluent can include a cellulose. In some embodiments, the cellulose can be microcrystalline cellulose. In some embodiments, the diluent can include Avicel PH-101. In some embodiment the diluent can have a nominal particle size between about 10 mm to about 20 mm, about 20 mm to about 30 mm, about 30 mm to about 40 mm, about 40 mm to about 50 mm, about 50 mm to about 60 mm, about 60 mm to about 70 mm, or about 70 mm to about 80 mm. In some embodiments, the diluent can have a nominal particle size of about 50 mm. In some embodiments, the diluent can have a moisture content between about 0.5% w/w to about 1% w/w, about 1% w/w to about 1.5% w/w, about 1.5% w/w to about 2% w/w, about 2% w/w to about 2.5% w/w, about 2.5% w/w to about 3% w/w, about 3.5% w/w to about 4% w/w, about 4% w/w to about 4.5% w/w, or about 4.5% w/w to about 5% w/w. In some embodiments, the diluent can have a moisture content between about 3% w/w to about 5% w/w. In some embodiments, the diluent can have a loose bulk density between about 0.1 g/cm³ to about 0.15 g/cm³, about 0.15 g/cm³ to about 0.2 g/cm³, about 0.2 g/cm³ to about 0.25 g/cm³, about 0.25 g/cm³ to about 0.3 g/cm³, about 0.3 g/cm³ to about 0.35 g/cm³, about 0.35 g/cm³ to about 0.4 g/cm³, about 0.4 g/cm³ to about 0.45 g/cm³, or about 0.45 g/cm³ to about 0.5 g/cm³. In some embodiments, the diluent can have a loose bulk density between about 0.26 g/cm³ to about 0.31 g/cm³.

In certain embodiments, Compound 1 a crystalline compound of formula:

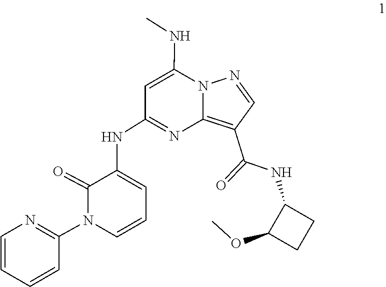 [SMILES: CNc1cc(Nc2cccn(-c3ccccn3)c2=O)nc2c(C(=O)N[C@@H]3CC[C@H]3OC)cnn12]

of polymorphic Form C, comprising an X-ray powder diffraction (XRPD) pattern comprising peaks at diffraction angles substantially as shown in FIG. 3.

In certain embodiments, Compound 1 can be amorphous. In some embodiments, Compound 1 have be as a hydrate. In some embodiments, Compound 1 can be as a monohydrate.

In some embodiments, Compound 1 can be in the form of a racemate:

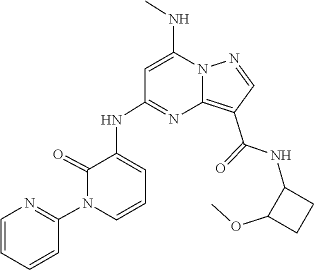 [SMILES: CNc1cc(Nc2cccn(-c3ccccn3)c2=O)nc2c(C(=O)NC3CCC3OC)cnn12]

or mixture of stereoisomers, or a solvate or a pharmaceutically acceptable salt thereof.

### Uses of Pharmaceutical Compositions

Compound 1 and compositions described herein are generally useful for the inhibition of kinase activity of one or more enzymes. In some embodiments the kinase inhibited by Compound 1 and methods described herein is TYK2.

TYK2 is a non-receptor tyrosine kinase member of the Janus kinase (JAKs) family of protein kinases. The mammalian JAK family consists of four members, TYK2, JAK1, JAK2, and JAK3. JAK proteins, including TYK2, are integral to cytokine signalling. TYK2 associates with the cytoplasmic domain of type I and type II cytokine receptors, as well as interferon types I and IT receptors, and is activated by those receptors upon cytokine binding. Cytokines implicated in TYK2 activation include interferons (e.g. IFN-α, IFN-β, IFN-κ, IFN-δ, IFN-ε, IFN-τ, IFN-ω, and IFN-ζ (also known as limitin), and interleukins (e.g. IL-4, IL-6, IL-10, IL-11, IL-12, IL-13, IL-22, IL-23, IL-27, IL-31, oncostatin M, ciliary neurotrophic factor, cardiotrophin 1, cardiotrophin-like cytokine, and LIF). Velasquez et al., “A protein kinase in the interferon α/β signalling pathway,” Cell (1992) 70:313; Stahl et al., “Association and activation of Jak-Tyk kinases by CNTF-LIF-OSM-IL-6β receptor components,” Science (1994) 263:92; Finbloom et al., “IL-10 induces the tyrosine phosphorylation of Tyk2 and Jak1 and the differential assembly of Stat1 and Stat3 complexes in human T cells and monocytes,” J. Immunol. (1995) 155:1079; Bacon et al., “Interleukin 12 (IL-12) induces tyrosine phosphorylation of Jak2 and Tyk2: differential use of Janus family kinases by IL-2 and IL-12,” J. Exp. Med. (1995) 181:399; Welham et al., “Interleukin-13 signal transduction in lymphohemopoietic cells: similarities and differences in signal transduction with interleukin-4 and insulin,” J. Biol. Chem. (1995) 270:12286; Parham et al., “A receptor for the heterodimeric cytokine IL-23 is composed of IL-12Rβ1 and a novel cytokine receptor subunit, IL-23R,” J. Immunol. (2002) 168:5699. The activated TYK2 then goes on to phosphorylate further signalling proteins such as members of the STAT family, including STAT1, STAT2, STAT4, and STAT6.

TYK2 activation by IL-23, has been linked to inflammatory bowel disease (IBD), Crohn's disease, and ulcerative colitis. Duerr et al., “A Genome-Wide Association Study Identifies IL23R as an Inflammatory Bowel Disease Gene,” Science (2006) 314:1461-1463. As the downstream effector of IL-23, TYK2 also plays a role in psoriasis, ankylosing spondylitis, and Behget's disease. Cho et al., “Genomics and the multifactorial nature of human auto-immune disease,” N. Engl. J. Med (2011) 365:1612-1623; Cortes et al., “Identification of multiple risk variants for ankylosing spondylitis through high-density genotyping of immune-related loci,” Nat. Genet. (2013) 45(7):730-738; Remmers et al., “Genome-wide association study identifies variants in the MHC class I, IL10, and IL23R-IL12RB2 regions associated with Behget's disease,” Nat. Genet. (2010) 42:698-702. A genome-wide association study of 2,622 individuals with psoriasis identified associations between disease susceptibility and TYK2. Strange et al., “A genome-wide association study identifies new psoriasis susceptibility loci and an interaction between HLA-C and ERAP1,” Nat. Genet. (2010) 42:985-992. Knockout or tyrphostin inhibition of TYK2 significantly reduces both IL-23 and IL-22-induced dermatitis. Ishizaki et al., “Tyk2 is a therapeutic target for psoriasis-like skin inflammation,” Intl. Immunol. (2013), doi: 10.1093/intimm/dxt062.

TYK2 also plays a role in respiratory diseases such as asthma, chronic obstructive pulmonary disease (COPD), lung cancer, and cystic fibrosis. Goblet cell hyperplasia (GCH) and mucous hypersecretion is mediated by IL-13-induced activation of TYK2, which in turn activates STAT6. Zhang et al., “Docking protein Gab2 regulates mucin expression and goblet cell hyperplasia through TYK2/STAT6 pathway,” FASEB J. (2012) 26:1-11. Decreased TYK2 activity leads to protection of joints from collagen antibody-induced arthritis, a model of human rheumatoid arthritis. Mechanistically, decreased Tyk2 activity reduced the production of Tₕ1/Tₕ17-related cytokines and matrix metalloproteases, and other key markers of inflammation. Ishizaki et al., “Tyk2 deficiency protects joints against destruction in anti-type II collagen antibody-induced arthritis in mice,” Intl. Immunol. (2011) 23(9):575-582.

TYK2 knockout mice showed complete resistance in experimental autoimmune encephalomyelitis (EAE, an animal model of multiple sclerosis (MS)), with no infiltration of CD4 T cells in the spinal cord, as compared to controls, suggesting that TYK2 is essential to pathogenic CD4-mediated disease development in MS. Oyamada et al., “Tyrosine Kinase 2 Plays Critical Roles in the Pathogenic CD4 T Cell Responses for the Development of Experimental Autoimmune Encephalomyelitis,” J. Immunol. (2009) 183:7539-7546. This corroborates earlier studies linking increased TYK2 expression with MS susceptibility. Ban et al., “Replication analysis identifies TYK2 as a multiple sclerosis susceptibility factor,” Eur J. Hum. Genet. (2009) 17:1309-1313. Loss of function mutation in TYK2, leads to decreased demyelination and increased remyelination of neurons, further suggesting a role for TYK2 inhibitors in the treatment of MS and other CNS demyelination disorders.

TYK2 is the sole signalling messenger common to both IL-12 and IL-23. TYK2 knockout reduced methylated BSA injection-induced footpad thickness, imiquimod-induced psoriasis-like skin inflammation, and dextran sulfate sodium or 2,4,6-trinitrobenzene sulfonic acid-induced colitis in mice.

Joint linkage and association studies of various type I IFN signalling genes with systemic lupus erythematosus (SLE, an autoimmune disorder), showed a strong, and significant correlation between loss of function mutations to TYK2 and decreased prevalence of SLE in families with affected members. Sigurdsson et al., “Polymorphisms in the Tyrosine Kinase 2 and Interferon Regulatory Factor 5 Genes Are Associated with Systemic Lupus Erythematosus,” Am. J. Hum. Genet. (2005) 76:528-537. Genome-wide association studies of individuals with SLE versus an unaffected cohort showed highly significant correlation between the TYK2 locus and SLE. Graham et al., “Association of NCF2, IKZF1, IRF8, IFIH1, and TYK2 with Systemic Lupus Erythematosus,” PLoS Genetics (2011) 7(10):e1002341.

TYK2 has been shown to play an important role in maintaining tumour surveillance and TYK2 knockout mice showed compromised cytotoxic T cell response, and accelerated tumour development. However, these effects were linked to the efficient suppression of natural killer (NK) and cytotoxic T lymphocytes, suggesting that TYK2 inhibitors would be highly suitable for the treatment of autoimmune disorders or transplant rejection. Although other JAK family members such as JAK3 have similar roles in the immune system, TYK2 has been suggested as a superior target because of its involvement in fewer and more closely related signalling pathways, leading to fewer off-target effects. Simma et al. “Identification of an Indispensable Role for Tyrosine Kinase 2 in CTL-Mediated Tumour Surveillance,” Cancer Res. (2009) 69:203-211.

However, paradoxically to the decreased tumour surveillance observed by Simma et al., studies in T-cell acute lymphoblastic leukaemia (T-ALL) indicate that T-ALL is highly dependent on IL-10 via TYK2 via STAT1-mediated signal transduction to maintain cancer cell survival through upregulation of anti-apoptotic protein BCL2. Knockdown of TYK2, but not other JAK family members, reduced cell growth. Specific activating mutations to TYK2 that promote cancer cell survival include those to the FERM domain (G36D, S47N, and R425H), the JH2 domain (V731I), and the kinase domain (E957D and R1027H). However, it was also identified that the kinase function of TYK2 is required for increased cancer cell survival, as TYK2 enzymes featuring kinase-dead mutations (M978Y or M978F) in addition to an activating mutation (E957D) resulted in failure to transform. Sanda et al. “TYK2-STAT1-BCL2 Pathway Dependence in T-Cell Acute Lymphoblastic Leukemia,” Cancer Disc. (2013) 3(5):564-577.

Thus, selective inhibition of TYK2 has been suggested as a suitable target for patients with IL-10 and/or BCL2-addicted tumours, such as 70% of adult T-cell leukaemia cases. Fontan et al. “Discovering What Makes STAT Signalling TYK in T-ALL,” Cancer Disc. (2013) 3:494-496.

TYK2 mediated STAT3 signalling has also been shown to mediate neuronal cell death caused by amyloid-β (Aβ) peptide. Decreased TYK2 phosphorylation of STAT3 following Aβ administration lead to decreased neuronal cell death, and increased phosphorylation of STAT3 has been observed in post-mortem brains of Alzheimer's patients. Wan et al. “Tyk/STAT3 Signalling Mediates β-Amyloid-Induced Neuronal Cell Death: Implications in Alzheimer's Disease,” J. Neurosci. (2010) 30(20):6873-6881.

Inhibition of JAK-STAT signalling pathways is also implicated in hair growth, and the reversal of the hair loss associated with alopecia areata. Xing et al., “Alopecia areata is driven by cytotoxic T lymphocytes and is reversed by JAK inhibition,” Nat. Med. (2014) 20: 1043-1049; Harel et al., “Pharmacologic inhibition of JAK-STAT signalling promotes hair growth,” Sci. Adv. (2015) 1(9):e1500973.

Accordingly, compounds that inhibit the activity of TYK2 are beneficial, especially those with selectivity over JAK2. Such compounds should deliver a pharmacological response that favorably treats one or more of the conditions described herein without the side-effects associated with the inhibition of JAK2.

Even though TYK2 inhibitors are known in the art, there is a continuing need to provide novel inhibitors having more effective or advantageous pharmaceutically relevant properties. For example, compounds with increased activity, selectivity over other JAK kinases (especially JAK2), and ADMET (absorption, distribution, metabolism, excretion, and/or toxicity) properties. Thus, in some embodiments, a pharmaceutical composition, formulation, or unit dosage form can include the Compound 1 inhibitor of TYK2 which shows selectivity over JAK2.

The activity of Compound 1 utilized as described herein as an inhibitor of TYK2, or a mutant thereof, may be assayed in vitro, in vivo or in a cell line. In vitro assays include assays that determine inhibition of either the phosphorylation activity and/or the subsequent functional consequences, or ATPase activity of activated TYK2, or a mutant thereof. Alternate in vitro assays quantitate the ability of the inhibitor to bind to TYK2. Inhibitor binding may be measured by radiolabeling the inhibitor prior to binding, isolating the inhibitor/TYK2 complex and determining the amount of radiolabel bound. Alternatively, inhibitor binding may be determined by running a competition experiment where new inhibitors are incubated with TYK2 bound to known radioligands. Representative in vitro and in vivo assays useful in assaying a TYK2 inhibitor include those described and disclosed in, e.g., each of which is herein incorporated by reference in its entirety.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Compound 1 is an inhibitor of TYK2 and is therefore useful for treating one or more disorders associated with activity of TYK2 or mutants thereof. Thus, in certain embodiments, a method for treating a TYK2-mediated disorder can include the step of administering to a patient in need thereof a pharmaceutical composition, formulation, or unit dosage form described herein, comprising Compound 1, or pharmaceutically acceptable salt, or hydrate thereof.

As used herein, the term “TYK2-mediated” disorders, diseases, and/or conditions as used herein means any disease or other deleterious condition in which TYK2 or a mutant thereof is known to play a role. Accordingly, another embodiment relates to treating or lessening the severity of one or more diseases in which TYK2, or a mutant thereof, is known to play a role. Such TYK2-mediated disorders include but are not limited to autoimmune disorders, inflammatory disorders, proliferative disorders, endocrine disorders, neurological disorders and disorders associated with transplantation.

In some embodiments, a method for treating one or more disorders is described herein, wherein the disorders are selected from autoimmune disorders, inflammatory disorders, proliferative disorders, endocrine disorders, neurological disorders, and disorders associated with transplantation, said method comprising administering to a patient in need thereof, a pharmaceutical composition comprising an effective amount of a pharmaceutical composition, formulation, or unit dosage form comprising Compound 1 as described herein.

In some embodiments, the disorder is an autoimmune disorder. In some embodiments the disorder is selected from type 1 diabetes, cutaneous lupus erythematosus, systemic lupus erythematosus, multiple sclerosis, psoriasis, Behget's disease, POEMS syndrome, Crohn's disease, ulcerative colitis, and inflammatory bowel disease.

In some embodiments, the disorder is an inflammatory disorder. In some embodiments, the inflammatory disorder is rheumatoid arthritis, asthma, chronic obstructive pulmonary disease, psoriasis, hepatomegaly, Crohn's disease, ulcerative colitis, inflammatory bowel disease.

In some embodiments, the disorder is a proliferative disorder. In some embodiments, the proliferative disorder is a hematological cancer. In some embodiments the proliferative disorder is a leukaemia. In some embodiments, the leukaemia is a T-cell leukaemia. In some embodiments the T-cell leukaemia is T-cell acute lymphoblastic leukaemia (T-ALL). In some embodiments the proliferative disorder is polycythemia vera, myelofibrosis, essential or thrombocytosis.

In some embodiments, the disorder is an endocrine disorder. In some embodiments, the endocrine disorder is polycystic ovary syndrome, Crouzon's syndrome, or type 1 diabetes.

In some embodiments, the disorder is a neurological disorder. In some embodiments, the neurological disorder is Alzheimer's disease.

In some embodiments the proliferative disorder is associated with one or more activating mutations in TYK2. In some embodiments, the activating mutation in TYK2 is a mutation to the FERM domain, the JH2 domain, or the kinase domain. In some embodiments the activating mutation in TYK2 is selected from G36D, S47N, R425H, V731I, E957D, and R1027H.

In some embodiments, the disorder is associated with transplantation. In some embodiments the disorder associated with transplantation is transplant rejection, or graft versus host disease.

In some embodiments the disorder is associated with type I interferon, IL-10, IL-12, or IL-23 signalling. In some embodiments the disorder is associated with type I interferon signalling. In some embodiments the disorder is associated with IL-10 signalling. In some embodiments the disorder is associated with IL-12 signalling. In some embodiments the disorder is associated with IL-23 signalling.

Formulations comprising Compound 1 as described herein are also useful in the treatment of inflammatory or allergic conditions of the skin, for example psoriasis, contact dermatitis, atopic dermatitis, alopecia areata, erythema multiforme, dermatitis herpetiformis, scleroderma, vitiligo, hypersensitivity angiitis, urticaria, bullous pemphigoid, lupus erythematosus, cutaneous lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Formulations comprising Compound 1 as described herein may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), cutaneous lupus erythematosus, systemic lupus erythematosus, rheumatoid arthritis, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine ophthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minimal change nephropathy), chronic granulomatous disease, endometriosis, leptospiriosis renal disease, glaucoma, retinal disease, ageing, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease which can be treated according to the methods described herein is selected from acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), and osteoarthritis.

In some embodiments the inflammatory disease which can be treated according to the methods described herein is a Tₕ1 or Tₕ17 mediated disease. In some embodiments the Tₕ17 mediated disease is selected from cutaneous lupus erythematosus, Systemic lupus erythematosus, Multiple sclerosis, and inflammatory bowel disease (including Crohn's disease or ulcerative colitis).

In some embodiments the inflammatory disease which can be treated according to the methods described herein is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

Furthermore, a formulation can include Compound 1 according to the definitions herein, for the preparation of a medicament for the treatment of an autoimmune disorder, an inflammatory disorder, or a proliferative disorder, or a disorder commonly occurring in connection with transplantation.

In some embodiments, a method of treating a disease selected from psoriasis, psoriatic arthritis, ulcerative colitis, Crohn's disease, and inflammatory bowel disease to a patient in need thereof, can include administering to a patient a therapeutically effective amount of a pharmaceutical composition, formulation, or unit dosage form described herein. In some embodiments, the disease is psoriasis. In some embodiments, the disease is psoriatic arthritis. In some embodiments, the disease is ulcerative colitis. In some embodiments, the disease is Crohn's disease. In some embodiments, the disease is inflammatory bowel disease.

In some embodiments, the administration is daily for a period between about 1 day to about 7 days, about 1 week to about 3 weeks, about 3 weeks to about 6 weeks, about 6 weeks to about 9 weeks, about 9 weeks to 12 weeks, about 12 weeks to about 15 weeks, or about 15 weeks to about 18 weeks.

In some embodiments, the method is directed to treatment of psoriasis and/or psoriatic arthritis.

In some embodiments, a mean decrease in psoriasis area severity index (PASI) of 25% or greater is achieved. In some embodiments, a mean decrease in psoriasis area severity index (PASI) of about 45% or greater is achieved. In some embodiments, a mean decrease in psoriasis area severity index (PASI) of about 25% to 50% is achieved. In some embodiments, a mean decrease in psoriasis area severity index (PASI) of about 50% to 75% is achieved. In some embodiments, a mean decrease in psoriasis area severity index (PASI) of about 75% to 100% is achieved.

In some embodiments, a Physician Global Assessment (PGA) of 0, 1, or 2 is achieved. In some embodiments, a Physician Global Assessment (PGA) of 0 or 1 is achieved. In some embodiments, a Physician Global Assessment (PGA) of 0 is achieved. In some embodiments, a Dermatology Life Quality Index (DLQI) score is reduced by an amount between about 1 to about 3, about 3 to about 6, about 6 to about 9, about 9 to about 12, about 12 to about 15, about 15 to about 18, about 18 to about 21, about 21 to about 24, about 24 to about 27, or about 27 to about 30.

In some embodiments, the patient's Body Surface Area (BSA) is reduced by a number of percentage points between about 1 to about 10, about 10 to about 20, about 20 to about 30, about 30 to about 40, about 40 to about 50, about 50 to about 60, about 60 to about 70, about 70 to about 80, about 80 to about 90, or about 90 to about 100.

In some embodiments, a reduction in pain numeric rating scale (NRS) of about 1, 2, 3, 4, 5, 6, 7, 8, 9, or 10 is achieved.

In some embodiments, a reduction in pruritus numeric rating scale (NRS) of about 1, 2, 3, 4, 5, 6, 7, 8, 9, or 10 is achieved.

In some embodiments, an inhibition of IL-12/18-induced IFNγ production between about 1% to about 10%, about 10% to about 20%, about 20% to about 30%, about 30% to about 40%, about 40% to about 50%, about 50% to about 60%, about 60% to about 70%, about 70% to about 80%, about 80% to about 90%, or about 90% to about 100%, is achieved.

In some embodiments, the patient has both psoriasis and psoriatic arthritis.

In some embodiments, the patient has psoriasis but not psoriatic arthritis.

In some embodiments, the psoriasis is moderate to severe.

In some embodiments, the psoriatic arthritis is moderate to severe.

In some embodiments, the patient has psoriatic arthritis.

In some embodiments, the method achieves at least an American College of Rheumatology (ACR) 20 response.

### Combination Therapies

Depending upon the particular condition, or disease, to be treated, additional therapeutic agents, which are normally administered to treat that condition, may be administered in combination with formulations comprising Compound 1 described herein. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

The formulations and compositions described herein may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinirole, pramipexole, bromocriptine, pergolide, trihexephendyl, and amantadine; agents for treating Multiple Sclerosis (MS) such as beta interferon (e.g., Avonex® and Rebifl), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies described herein, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound described herein in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with methods described herein. For example, a combination described herein may be administered with another therapeutic agent simultaneously or sequentially in separate unit dosage forms or together in a single unit dosage form.

The amount of additional therapeutic agent present in the compositions described herein will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, a composition can include Compound 1 and one or more additional therapeutic agents. The therapeutic agent may be administered together with Compound 1, or may be administered prior to or following administration of Compound 1. Suitable therapeutic agents are described in further detail below. In certain embodiments, Compound 1 may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, Compound 1 may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, a method of treating an inflammatory disease, disorder or condition by administering to a patient in need thereof a formulation can include Compound 1 as described herein and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvise® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin®HFA, Proventil®HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, a method of treating rheumatoid arthritis can include administering to a patient in need thereof a formulation comprising Compound 1 and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, a method of treating osteoarthritis can include administering to a patient in need thereof a formulation comprising Compound 1 and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, a method of treating cutaneous lupus erythematosus or systemic lupus erythematosus can include administering to a patient in need thereof a formulation comprising Compound 1 and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, a method of treating Crohn's disesase, ulcerative colitis, or inflammatory bowel disease can include administering to a patient in need thereof a formulation comprising Compound 1 and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, a method of treating asthma can include administering to a patient in need thereof a formulation comprising Compound 1 and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, a method of treating COPD can include administering to a patient in need thereof a formulation comprising Compound 1 and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In another embodiment, a method of treating a hematological malignancy can include administering to a patient in need thereof a formulation comprising Compound 1 and one or more additional therapeutic agents selected from rituximab (Rituxan®), cyclophosphamide (Cytoxan®), doxorubicin (Hydrodaunorubicin®), vincristine (Oncovin®), prednisone, a hedgehog signalling inhibitor, a BTK inhibitor, a JAK/pan-JAK inhibitor, a PI3K inhibitor, a SYK inhibitor, and combinations thereof.

In another embodiment, a method of treating a solid tumour can include administering to a patient in need thereof a formulation comprising Compound 1 and one or more additional therapeutic agents selected from rituximab (Rituxan®), cyclophosphamide (Cytoxan®), doxorubicin (Hydrodaunorubicin®), vincristine (Oncovin®), prednisone, a hedgehog signalling inhibitor, a BTK inhibitor, a JAK/pan-JAK inhibitor, a PI3K inhibitor, a SYK inhibitor, and combinations thereof.

In another embodiment, a method of treating a hematological malignancy can include administering to a patient in need thereof a formulation comprising Compound 1 and a Hedgehog (Hh) signalling pathway inhibitor. In some embodiments, the hematological malignancy is DLBCL (Ramirez et al “Defining causative factors contributing in the activation of hedgehog signalling in diffuse large B-cell lymphoma” Leuk. Res. (2012), published online July 17, and incorporated herein by reference in its entirety).

In another embodiment, a method of treating diffuse large B-cell lymphoma (DLBCL) can include administering to a patient in need thereof a formulation comprising Compound 1 and one or more additional therapeutic agents selected from rituximab (Rituxan®), cyclophosphamide (Cytoxan®), doxorubicin (Hydrodaunorubicin®), vincristine (Oncovin®), prednisone, a hedgehog signalling inhibitor, and combinations thereof.

In another embodiment, a method of treating multiple myeloma can include administering to a patient in need thereof a compound of a formulation comprising Compound 1 and one or more additional therapeutic agents selected from bortezomib (Velcade®), and dexamethasone (Decadron®), a hedgehog signalling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, a method of treating or lessening the severity of a disease can include administering to a patient in need thereof a formulation comprising Compound 1 and a BTK inhibitor, wherein the disease is selected from inflammatory bowel disease, arthritis, cutaneous lupus erythematosus, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukaemia, acute lymphocytic leukaemia, B-cell prolymphocytic leukaemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukaemia, or lymphomatoid granulomatosis, breast cancer, prostate cancer, or cancer of the mast cells (e.g., mastocytoma, mast cell leukaemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, cutaneous lupus erythematosus, systemic lupus erythematosus (SLE), rheumatoid arthritis, psoriatic arthritis, juvenile arthritis, osteoarthritis, chronic idiopathic thrombocytopenic purpura, myasthenia gravis, Hashimoto's thyroiditis, atopic dermatitis, degenerative joint disease, vitiligo, autoimmune hypopituitarism, scleroderma, mycosis fungoides, and acute inflammatory responses (such as acute respiratory distress syndrome and ischemia/reperfusion injury).

In another embodiment, a method of treating or lessening the severity of a disease can include administering to a patient in need thereof a formulation comprising Compound 1 disclosed herein, and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukaemia (CML), chronic lymphocytic leukaemia (CLL), liver disease, pathologic immune conditions involving T cell activation, a cardiovascular disorder, and a CNS disorder.

In another embodiment, a method of treating or lessening the severity of a disease can include administering to a patient in need thereof a formulation comprising Compound 1 disclosed herein, and a PI3K inhibitor, wherein the disease is selected from benign or malignant tumour, carcinoma or solid tumour of the brain, kidney (e.g., renal cell carcinoma (RCC)), liver, adrenal gland, bladder, breast, stomach, gastric tumours, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumour of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukaemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), cutaneous lupus erythematosus, systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g., ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g., including idiopathic nephrotic syndrome or minimal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, a method of treating or lessening the severity of a disease can include administering to a patient in need thereof a formulation comprising Compound 1 disclosed herein, and a Bcl-2 inhibitor, wherein the disease is an inflammatory disorder, an autoimmune disorder, a proliferative disorder, an endocrine disorder, a neurological disorder, or a disorder associated with transplantation. In some embodiments, the disorder is a proliferative disorder, lupus, or lupus nephritis. In some embodiments, the proliferative disorder is chronic lymphocytic leukaemia, diffuse large B-cell lymphoma, Hodgkin's disease, small-cell lung cancer, non-small-cell lung cancer, myelodysplastic syndrome, lymphoma, a hematological neoplasm, or solid tumour.

In some embodiments, a method of treating or lessening the severity of a disease, can include administering to a patient in need thereof a TYK2 pseudokinase (JH2) domain binding compound and a TYK2 kinase (JH1) domain binding compound. In some embodiments, the disease is an autoimmune disorder, an inflammatory disorder, a proliferative disorder, an endocrine disorder, a neurological disorder, or a disorder associated with transplantation. In some embodiments the JH2 is binding Compound 1. Other suitable JH2 domain binding compounds include those described in WO2014074660A1, WO2014074661A1, WO2015089143A1, the entirety of each of which is incorporated herein by reference. Suitable JH1 domain binding compounds include those described in WO2015131080A1, the entirety of which is incorporated herein by reference.

Compound 1 compositions may be administered using any amount and any route of administration effective for treating or lessening the severity of an autoimmune disorder, an inflammatory disorder, a proliferative disorder, an endocrine disorder, a neurological disorder, or a disorder associated with transplantation. The exact amount required will vary from subject to subject, depending on the species, age, and general condition of the subject, the severity of the infection, the particular agent, its mode of administration, and the like. Compound 1 is preferably formulated in unit dosage form for ease of administration and uniformity of dosage. The expression “unit dosage form” as used herein refers to a physically discrete unit of agent appropriate for the patient to be treated. It will be understood, however, that the total daily usage of the compound and compositions described herein will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient,” as used herein, means an animal, preferably a mammal, and most preferably a human.

Pharmaceutically acceptable compositions described herein can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, the compound described herein may be administered orally or parenterally at dosage levels of about 0.01 mg/kg to about 50 mg/kg and preferably from about 0.01 mg/kg to about 5 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a Compound 1, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of Compound 1 release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing Compound 1 with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compound can also be in micro-encapsulated form with one or more excipients as noted above. In some embodiments, hydroxypropyl methyl cellulose (HMPC) capsules encapsulate the composition or formulation described herein. In some embodiments, the capsules are size 2 hard Swedish orange HPMC capsules. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a formulation comprising Compound 1 described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of the compositions and formulations described herein. Additionally, the formulation and compositions described herein can be used as of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, a method of inhibiting protein kinase activity in a biological sample can include the step of contacting said biological sample with a formulation comprising Compound 1 described herein.

According to another embodiment, a method of inhibiting TYK2, or a mutant thereof, activity in a biological sample can include the step of contacting said biological sample with Compound 1 described herein, or a composition comprising said compound. In certain embodiments, a method of irreversibly inhibiting TYK2, or a mutant thereof, activity in a biological sample can include the step of contacting said biological sample with a formulation comprising Compound 1 described herein.

In another embodiment, a method of selectively inhibiting TYK2 over one or more of JAK1, JAK2, and JAK3 is described herein. In some embodiments, a formulation comprising Compound 1 described herein is more than 2-fold selective over JAK1/2/3. In some embodiments, a compound described herein is more than 5-fold selective over JAK1/2/3. In some embodiments, the compound described herein is more than 10-fold selective over JAK1/2/3. In some embodiments, the compound described herein is more than 50-fold selective over JAK1/2/3. In some embodiments, the compound described herein is more than 100-fold selective over JAK1/2/3.

The term “biological sample,” as used herein, includes, without limitation, cell cultures or extracts thereof; biopsied material obtained from a mammal or extracts thereof; and blood, saliva, urine, feces, semen, tears, or other body fluids or extracts thereof.

Inhibition of TYK2 (or a mutant thereof) activity in a biological sample is useful for a variety of purposes that are known to one of skill in the art. Examples of such purposes include, but are not limited to, blood transfusion, organ-transplantation, biological specimen storage, and biological assays.

Another embodiment relates to a method of inhibiting protein kinase activity in a patient including the step of administering to said patient a formulation comprising Compound 1 described herein.

According to another embodiment, a method of inhibiting activity of TYK2, or a mutant thereof, in a patient can include the step of administering to said patient a formulation comprising Compound 1 described herein. According to certain embodiments, a method of reversibly or irreversibly inhibiting one or more of TYK2, or a mutant thereof, activity in a patient can include the step of administering to said patient a formulation comprising Compound 1 described herein. In other embodiments, a method for treating a disorder mediated by TYK2, or a mutant thereof, in a patient in need thereof, can include the step of administering to said patient a formulation comprising Compound 1 described herein. Such disorders are described in detail herein.

Depending upon the particular condition, or disease, to be treated, additional therapeutic agents that are normally administered to treat that condition, may also be present in the compositions described herein. As used herein, additional therapeutic agents that are normally administered to treat a particular disease, or condition, are known as “appropriate for the disease, or condition, being treated.”

A formulation comprising Compound 1 described herein may also be used in combination with other therapeutic compounds. In some embodiments, the other therapeutic compounds are antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal©); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™ Letrozole is marketed under the trade names Femara™ or Femar™. Aminoglutethimide is marketed under the trade name Orimeten™. A combination of the the composition or formulation described herein comprising a chemotherapeutic agent which is an aromatase inhibitor is particularly useful for the treatment of hormone receptor positive tumours, such as breast tumours.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the composition or formulation described herein comprising a chemotherapeutic agent which is an antiestrogen is particularly useful for the treatment of estrogen receptor positive tumours, such as breast tumours.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g., in the form as it is marketed, e.g., under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g., under the trademark Carboplat™. Oxaliplatin can be administered, e.g., in the form as it is marketed, e.g., under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; 1sis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signalling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this compositions, formulations and methods described herein include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

Further examples of BTK inhibitory compounds, and conditions treatable by such compounds in combination with compounds described herein can be found in WO2008039218 and WO2011090760, the entirety of which are incorporated herein by reference.

Further examples of SYK inhibitory compounds, and conditions treatable by such compounds in combination with compounds described herein can be found in WO2003063794, WO2005007623, and WO2006078846, the entirety of which are incorporated herein by reference.

Further examples of PI3K inhibitory compounds, and conditions treatable by such compounds in combination with compounds described herein can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds described herein can be found in WO2009114512, WO2008109943, WO2007053452, WO2000142246, and WO2007070514, the entirety of which are incorporated herein by reference.

Further anti-angiogenic compounds include compounds having another mechanism for their activity, e.g., unrelated to protein or lipid kinase inhibition e.g., thalidomide (Thalomid™) and TNP-470.

Examples of proteasome inhibitors useful for use in combination with a formulation comprising Compound 1 described herein include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g., inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™ Pamidronic acid is marketed under the trade name Aredia™. Alendronic acid is marketed under the trade name Fosamax™. Ibandronic acid is marketed under the trade name Bondranat™ Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms,” such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g., hydroxamate peptidomimetic inhibitor batimastat and its orally bioavailable analogue marimastat (BB-2516), prinomastat (AG3340), metastat (NSC 683551) BMS-279251. BAY 12-9566, TAA211. MMJ270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; ALK inhibitors, which are compounds which target, decrease or inhibit anaplastic lymphoma kinase, and Bcl-2 inhibitors.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan©), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds described herein can be used in combination with standard leukaemia therapies, especially in combination with therapies used for the treatment of AML. In particular, a formulation comprising Compound 1 described herein can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412. In some embodiments, a method of treating AML associated with an ITD and/or D835Y mutation, can include administering a formulation comprising Compound 1 described herein together with a one or more FLT3 inhibitors. In some embodiments, the FLT3 inhibitors are selected from quizartinib (AC220), a staurosporine derivative (e.g., midostaurin or lestaurtinib), sorafenib, tandutinib, LY-2401401, LS-104, EB-10, famitinib, NOV-110302, NMS-P948, AST-487, G-749, SB-1317, S-209, SC-110219, AKN-028, fedratinib, tozasertib, and sunitinib. In some embodiments, the FLT3 inhibitors are selected from quizartinib, midostaurin, lestaurtinib, sorafenib, and sunitinib.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumour cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The formulation comprising Compound 1 described herein is also useful as a co-therapeutic compound for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A formulation comprising Compound 1 described herein may be mixed with the other drug substance in a fixed pharmaceutical composition or it may be administered separately, before, simultaneously with or after the other drug substance. Accordingly, a combination of a formulation can include Compound 1 described herein with an anti-inflammatory, bronchodilatory, antihistamine or anti-tussive drug substance, said compound described herein and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C. CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden),V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds described herein with anti-inflammatory drugs are those with antagonists of chemokine receptors, e.g., CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g., Patents International (e.g. IMS World Publications).

A formulation comprising Compound 1 described herein may also be used in combination with known therapeutic processes, for example, the administration of hormones or radiation. In certain embodiments, a provided compound is used as a radiosensitizer, especially for the treatment of tumours which exhibit poor sensitivity to radiotherapy.

A formulation comprising Compound 1 described herein can be administered alone or in combination with one or more other therapeutic compounds, possible combination therapy taking the form of fixed combinations or the administration of Compound 1 as a formulation described herein and one or more other therapeutic compounds being staggered or given independently of one another, or the combined administration of fixed combinations and one or more other therapeutic compounds. Compound 1 can besides or in addition be administered especially for tumour therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumour regression, or even chemopreventive therapy, for example in patients at risk.

Those additional agents may be administered separately from an inventive compound-containing composition, as part of a multiple dosage regimen. Alternatively, those agents may be part of a single dosage form, mixed together with Compound 1 in a single formulation or composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with methods described herein. For example, Compound 1 may be administered with another therapeutic agent simultaneously or sequentially in separate unit dosage forms or together in a single unit dosage form. Accordingly, a single unit dosage form can include Compound 1, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions described herein should be formulated so that a dosage of between 0.01-10 mg/kg body weight/day of Compound 1 or a solvate or pharmaceutically acceptable salt thereof can be administered.

In those compositions which comprise an additional therapeutic agent, that additional therapeutic agent and Compound 1 may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions comprising Compound 1 will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Compound 1 and pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound described herein are another embodiment of the compositions and formulations described herein.

In some embodiments, a medicament can include at least Compound 1 formulated as described herein.

### Pharmaceutical Formulation Components

The compositions described herein are administered using any amount and any route of administration effective for treating or lessening the severity of a disease described above. The exact amount required will vary from subject to subject, depending on the species, age, and general condition of the subject, the severity of the infection, the particular agent, its mode of administration, and the like. Compound 1 is preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions described herein will be decided by the attending physician within the scope of sound medical judgment. The specific effective dose level for any particular patient or organism (e.g., and feline, canine, bovine, equine, porcine, or aves) will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions described herein can be administered to humans and other animals orally, rectally, parenterally, intracisternally, intrathecally, transdermally, transmucosally, opthalmically, via inhalation, intravaginally, intraperitoneally, topically (as by powders, ointments, or drops), buccally, intranasally, as an oral or nasal spray, or the like, depending on the severity of the disease being treated. In certain embodiments, the compounds described herein are administered orally or parenterally at dosage levels of about 0.01 mg/kg to about 50 mg/kg and for example from about 1 mg/kg to about 25 mg/kg, of subject body weight per day, one or more times a day, to obtain the desired therapeutic effect.

A unit dosage form described herein can be formulated for oral administration. Pharmaceutical compositions/formulations that are suitable for oral administration can be provided as discrete dosage forms, such as, but not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In some embodiments, such dosage forms contain predetermined amounts of active ingredients, and may be prepared by methods of pharmacy known to those skilled in the art. See generally, Remington's Pharmaceutical Sciences, 18^{th} ed., Mack Publishing, Easton Pa. (1990). As used herein, oral administration also includes buccal, lingual, and sublingual administration.

In some embodiments, the formulation further comprises one or more pharmaceutically acceptable excipients or carriers.

A person of ordinary skill would recognize that pharmaceutical formulation ingredients may serve multiple purposes within a formulation. Accordingly, a person of ordinary skill would recognize that certain formulation components may be classified according to multiple functions (e.g., a component may be both a filler and a binder).

In some embodiments, a unit dosage form provided herein are prepared by combining the active ingredients in an intimate admixture with one or more pharmaceutically acceptable excipients or carriers, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide, according to conventional pharmaceutical compounding techniques. Excipients or carriers can take a wide variety of forms depending on the form of preparation desired for administration. For example, excipients or carriers suitable for use in oral liquid or aerosol dosage forms include, but are not limited to, water, glycols, oils, alcohols, flavoring agents, preservatives, and coloring agents. Examples of excipients or carriers suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In some embodiments, the active ingredient, such as Compound 1 or a pharmaceutically acceptable salt thereof, is incorporated into the pharmaceutical composition as spray-dried powder or granules. The use of spray-drying to produce powders from fluid feed stocks is well known, with applications ranging from powdered milk to bulk chemicals and pharmaceuticals. See U.S. Pat. No. 4,187,617 and Mujumbar et al., 91 Drying, pages 56-73 (1991). The use of spray-drying to form solid amorphous dispersions of drugs and concentration-enhancing polymers is also known. See commonly owned European Patent Applications Nos. 0 901 786, 1 027 886, 1 027 887, 1 027 888, and commonly owned PCT Applications Nos. WO 00/168092 and WO 00/168055, each of which is hereby incorporated by reference. A typical spray-drying apparatus comprises a drying chamber, atomizing means for atomizing a solvent-containing liquid feed into the drying chamber, a source of heated drying gas directed into the drying chamber and dried product collection means for separating the dried product from the cooled drying gas and vaporized solvent stream following its exit from the drying chamber. Examples of such apparatus include Niro Models PSD-1, PSD-2 and PSD-4 (Niro A/S, Soeborg, Denmark).

The spray-dried powder or granules generally include the active compound in combination with a polymer such as a concentration-enhancing polymer. One class of polymers suitable for use with the compositions and formulations described herein comprises non-ionizable (neutral) non-cellulosic polymers. Exemplary polymers include: vinyl polymers and copolymers having at least one substituent selected from the group consisting of hydroxyl, alkylacyloxy, and cyclicamido; polyvinyl alcohols that have at least a portion of their repeat units in the unhydrolyzed (vinyl acetate) form; polyvinyl alcohol polyvinyl acetate copolymers; polyvinyl pyrrolidone; and polyethylene polyvinyl alcohol copolymers; and polyoxyethylene-polyoxypropylene copolymers.

Exemplary neutral non-cellulosic polymers are comprised of vinyl copolymers of at least one hydrophilic, hydroxyl-containing repeat unit and at least one hydrophobic, alkyl- or aryl-containing repeat unit. Such neutral vinyl copolymers are termed “amphiphilic hydroxyl-functional vinyl copolymers.” Amphiphilic hydroxyl-functional vinyl copolymers are believed to provide high concentration enhancements due to the amphiphilicity of these copolymers which provide both sufficient hydrophobic groups to interact with the hydrophobic, low-solubility drugs and also sufficient hydrophilic groups to have sufficient aqueous solubility for good dissolution. The copolymeric structure of the amphiphilic hydroxyl-functional vinyl copolymers also allows their hydrophilicity and hydrophobicity to be adjusted to maximize performance with a specific low-solubility drug.

Another class of polymers suitable for use with the compositions and formulations described herein comprises ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGIT™ series manufactured by Rohm Tech Inc., of Malden, Mass.; amine-functionalized polyacrylates and polymethacrylates; proteins such as gelatin and albumin; and carboxylic acid functionalized starches such as starch glycolate.

Non-cellulosic polymers that are amphiphilic are copolymers of a relatively hydrophilic and a relatively hydrophobic monomer. Examples include acrylate and methacrylate copolymers. Exemplary commercial grades of such copolymers include the EUDRAGIT™ series, which are copolymers of methacrylates and acrylates.

An additional class of polymers comprises ionizable and neutral (or non-ionizable) cellulosic polymers with at least one ester- and/or ether-linked substituent in which the polymer has a degree of substitution of at least 0.05 for each substituent. It should be noted that in the polymer nomenclature used herein, ether-linked substituents are recited prior to “cellulose” as the moiety attached to the ether group; for example, “ethylbenzoic acid cellulose” has ethoxybenzoic acid substituents. Analogously, ester-linked substituents are recited after “cellulose” as the carboxylate; for example, “cellulose phthalate” has one carboxylic acid of each phthalate moiety ester-linked to the polymer and the other carboxylic acid unreacted.

It should also be noted that a polymer name such as “cellulose acetate phthalate” (CAP) refers to any of the family of cellulosic polymers that have acetate and phthalate groups attached via ester linkages to a significant fraction of the cellulosic polymer's hydroxyl groups. Generally, the degree of substitution of each substituent group can range from 0.05 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate-substituted, the phthalate degree of substitution is 3. Also included within each polymer family type are cellulosic polymers that have additional substituents added in relatively small amounts that do not substantially alter the performance of the polymer.

Amphiphilic cellulosics comprise polymers in which the parent cellulosic polymer has been substituted at any or all of the 3 hydroxyl groups present on each saccharide repeat unit with at least one relatively hydrophobic substituent. Hydrophobic substituents may be essentially any substituent that, if substituted to a high enough level or degree of substitution, can render the cellulosic polymer essentially aqueous-insoluble. Examples of hydrophobic substituent include ether-linked alkyl groups such as methyl, ethyl, propyl, butyl, etc.; or ester-linked alkyl groups such as acetate, propionate, butyrate, etc.; and ether- and/or ester-linked aryl groups such as phenyl, benzoate, or phenylate. Hydrophilic regions of the polymer can be either those portions that are relatively unsubstituted, since the unsubstituted hydroxyls are themselves relatively hydrophilic, or those regions that are substituted with hydrophilic substituents. Hydrophilic substituents include ether- or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether- or ester-linked ionizable groups such as carboxylic acids, thiocarboxylic acids, substituted phenoxy groups, amines, phosphates or sulfonates.

One class of cellulosic polymers comprises neutral polymers, meaning that the polymers are substantially non-ionizable in aqueous solution. Such polymers contain non-ionizable substituents, which may be either ether-linked or ester-linked. Exemplary ether-linked non-ionizable substituents include: alkyl groups, such as methyl, ethyl, propyl, butyl, etc.; hydroxy alkyl groups such as hydroxymethyl, hydroxyethyl, hydroxypropyl, etc.; and aryl groups such as phenyl. Exemplary ester-linked non-ionizable substituents include: alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Exemplary nonionizable cellulosic polymers that may be used as the polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

An exemplary class of neutral cellulosic polymers are those that are amphiphilic. Exemplary polymers include hydroxypropyl methyl cellulose and hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

A particular class of cellulosic polymers comprises polymers that are at least partially ionizable at physiologically relevant pH and include at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulphonate ethoxy. Exemplary ester-linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer aqueous soluble at least at pH values where any ionizable groups are ionized. In some cases, the aromatic substituent may itself be ionizable, such as phthalate or trimellitate substituents.

Exemplary cellulosic polymers that are at least partially-ionized at physiologically relevant pHs include: hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, carboxymethyl ethyl cellulose, ethyl carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Exemplary cellulosic polymers that meet the definition of amphiphilic, having hydrophilic and hydrophobic regions include polymers such as cellulose acetate phthalate and cellulose acetate trimellitate where the cellulosic repeat units that have one or more acetate substituents are hydrophobic relative to those that have no acetate substituents or have one or more ionized phthalate or trimellitate substituents.

A further subset of cellulosic ionizable polymers are those that possess both a carboxylic acid functional aromatic substituent and an alkylate substituent and thus are amphiphilic. Exemplary polymers include cellulose acetate phthalate, methyl cellulose acetate phthalate, ethyl cellulose acetate phthalate, hydroxypropyl cellulose acetate phthalate, hydroxylpropyl methyl cellulose phthalate, hydroxypropyl methyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Another subset of cellulosic ionizable polymers are those that possess a non-aromatic carboxylate substituent. Exemplary polymers include hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate and carboxymethyl ethyl cellulose. Of these cellulosic polymers that are at least partially ionized at physiologically relevant pHs, for example, hydroxypropyl methyl cellulose acetate succinate, hydroxypropyl methyl cellulose phthalate, cellulose acetate phthalate, cellulose acetate trimellitate and carboxymethyl ethyl cellulose. In some embodiments, the polymer is hydroxypropyl methyl cellulose acetate succinate (HPMCAS).

Another class of polymers consists of neutralized acidic polymers. By “neutralized acidic polymer” is meant any acidic polymer for which a significant fraction of the “acidic moieties” or “acidic substituents” have been “neutralized”; that is, exist in their deprotonated form. By “neutralized acidic cellulosic polymers” is meant any cellulosic “acidic polymer” in which a significant fraction of the “acidic moieties” or “acidic substituents” have been “neutralized.” By “acidic polymer” is meant any polymer that possesses a significant number of acidic moieties. In general, a significant number of acidic moieties would be greater than or equal to about 0.1 milliequivalents of acidic moieties per gram of polymer. “Acidic moieties” include any functional groups that are sufficiently acidic that, in contact with or dissolved in water, can at least partially donate a hydrogen cation to water and thus increase the hydrogen-ion concentration. This definition includes any functional group or “substituent,” as it is termed when the functional group is covalently attached to a polymer that has a pKₐ of less than about 10. Exemplary classes of functional groups that are included in the above description include carboxylic acids, thiocarboxylic acids, phosphates, phenolic groups, and sulfonates. Such functional groups may make up the primary structure of the polymer such as for polyacrylic acid, but more generally are covalently attached to the backbone of the parent polymer and thus are termed “substituents.”

The amount of concentration-enhancing polymer relative to the amount of drug (Compound 1) present in the spray-dried dispersions depends on the drug and concentration-enhancing polymer and may vary widely from a drug-to-polymer weight ratio of 0.01 to 5. However, in most cases, except when the drug dose is quite low, e.g., 25 mg or less, it is typical that the drug-to-polymer ratio is greater than 0.05 and less than 2.5 and often the enhancement in drug concentration or relative bioavailability is observed at drug-to-polymer ratios of 1 or less or for some drugs even 0.2 or less. In cases where the drug dose is about 25 mg or less, the drug-to-polymer weight ratio may be significantly less than 0.05. In general, regardless of the dose, enhancements in drug concentration or relative bioavailability increase with decreasing drug-to-polymer weight ratio. However, due to the practical limits of keeping the total mass of a tablet, capsule or suspension low, it is often desirable to use a relatively high drug-to-polymer ratio as long as satisfactory results are obtained. The maximum drug:polymer ratio that yields satisfactory results varies from drug to drug and is best determined in the in dissolution tests described below.

A spray-dried solid as described herein can be a solid dispersion that contains a compound described herein and a pharmaceutically acceptable polymer. Certain compounds described herein generally have low aqueous solubility, and their absorption in vivo is dissolution-rate limited. A solid dispersion containing a compound can increase the compound solubility/dissolution, thereby improving the bioavailability of the compound.

The term “solid dispersion” herein refers to the dispersion of a pharmaceutically active ingredient, e.g., the compound described herein, in an inert polymer matrix at solid state. A solid dispersion can be prepared by methods well known in the art, e.g., spray-drying or hot-melt extrusion. The matrix can be either crystalline or amorphous. A solid dispersion contains a co-precipitate of a pharmaceutically active ingredient and one or more water-soluble polymers, in which the pharmaceutically active ingredient is dispersed uniformly within a polymer matrix formed from the polymers. The pharmaceutically active ingredient can be present in an amorphous state, a crystalline dispersed form, or a combination thereof. It can also be finely dispersed or dissolved as single molecules in the polymer matrix. The solid dispersion is typically prepared by a spray-drying method or a hot-melt extrusion method.

The method for preparing the solid dispersion includes steps of (i) mixing a compound described herein and a polymer in an organic solvent to provide a feeder solution and (ii) spray-drying the feeder solution through a nozzle as a fine spray into a chamber where the solvent is evaporated quickly to generate particles containing the compound and polymer. Following formation of a solid dispersion, the resulting spray-dried particle can undergo a secondary drying step to remove residual solvents. The secondary drying step can take place in a static dryer or an agitated dryer. Gas, humidified gas, vacuum can be applied to the secondary drying step and such application is useful in more rapidly removing residual solvents that remain in the spray-dried particle.

Any organic solvent that can easily dissolve or disperse the compound and the polymer described above can be used. Examples of the organic solvent include lower carbon-number alcohols, e.g., methanol, ethanol, propanol, and isopropanol; ketones, e.g., methylethyl ketone and butanone; and a combination thereof.

In some embodiments, the pharmaceutically acceptable excipients and carriers are selected from fillers, binders, diluents, disintegrants, glidants, and lubricants.

In some embodiments, a capsule or tablet can include a provided pharmaceutical composition in the form of a solid dosage form. In some embodiments, the solid dosage form can be a capsule. In some embodiments, the solid dosage form can be a tablet.

In certain embodiments, the dosage form is a tablet, wherein the tablet is manufactured using standard, art-recognized tablet processing procedures and equipment. In certain embodiments, the method for forming the tablets is direct compression of a powdered, crystalline and/or granular composition comprising a solid form provided herein, alone or in combination with one or more excipients or carriers, such as, for example, carriers, additives, polymers, or the like. In certain embodiments, as an alternative to direct compression, the tablets may be prepared using wet granulation or dry granulation processes. In certain embodiments, the tablets are molded rather than compressed, starting with a moist or otherwise tractable material. In certain embodiments, compression and granulation techniques are used.

In certain embodiments, the dosage form is a capsule, wherein the capsules may be manufactured using standard, art-recognized capsule processing procedures and equipment. In certain embodiments, soft gelatin capsules may be prepared in which the capsules contain a mixture comprising a solid form provided herein and vegetable oil or non-aqueous, water miscible materials, such as, for example, polyethylene glycol and the like. In certain embodiments, hard gelatin capsules may be prepared containing granules of solid forms provided herein in combination with a solid pulverulent carrier, such as, for example, lactose, saccharose, sorbitol, mannitol, potato starch, corn starch, amylopectin, cellulose derivatives, or gelatin. In certain embodiments, a hard gelatin capsule shell may be prepared from a capsule composition comprising gelatin and a small amount of plasticizer such as glycerol. In certain embodiments, as an alternative to gelatin, the capsule shell may be made of a carbohydrate material. In certain embodiments, the capsule composition may additionally include polymers, colorings, flavorings and opacifiers as required. In certain embodiments, the capsule comprises HPMC.

In some embodiments, the pharmaceutical composition comprises one or more fillers. In certain embodiments, the filler is selected from ammonium aliginate, calcium carbonate, calcium lactate, calcium phosphate, calcium silicate, calcium sulfate, cellulose acetate, compressible sugar (e.g., lactose, glucose, and sucrose), corn starch, dextrates, erythritol, ethyl cellulose, glyceryl palmitostearate, isomalt, kaolin, magnesium carbonate, magnesium oxide, maltodextrin, medium-chain triglycerides, microcrystalline cellulose, pre-gelatinized starch, polydextrose, polymethacrylates, silicic acid, simethicone, sodium alginate, sodium chloride, sorbitol, starch, sugar spheres, sulfobutylether β-cyclodextrin, talc, tragacanth, trehalose, and xylitol, or a combination thereof.

In some embodiments, the filler is selected from talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

In some embodiments, the filler is microcrystalline cellulose. In some embodiments, the filler is lactose. In some embodiments, the filler is starch. In some embodiments, the filler is a combination of starch and lactose. In some embodiments, the filler is a combination of lactose and microcrystalline cellulose. In some embodiments, the filler is a combination of two or three components recited above. In some embodiments, the filler comprises at least microcrystalline cellulose, lactose, and mannitol.

In certain embodiments, dosage forms provided herein comprise one or more diluents. Diluents may be used, e.g., to increase bulk so that a practical size tablet or capsule is ultimately provided. Suitable diluents include dicalcium phosphate, calcium sulfate, lactose, cellulose, kaolin, mannitol, sodium chloride, dry starch, microcrystalline cellulose (e.g., AVICEL), microfine cellulose, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT), potassium chloride, sodium chloride, sorbitol and talc, among others. Diluents also include, e.g., ammonium alginate, calcium carbonate, calcium phosphate, calcium sulfate, cellulose acetate, compressible sugar, confectioner's sugar, dextrates, dextrin, dextrose, erythritol, ethylcellulose, fructose, fumaric acid, glyceryl palmitostearate, isomalt, kaolin, lacitol, lactose, mannitol, magnesium carbonate, magnesium oxide, maltodextrin, maltose, medium-chain triglycerides, microcrystalline cellulose, microcrystalline silicified cellulose, powered cellulose, polydextrose, polymethylacrylates, simethicone, sodium alginate, sodium chloride, sorbitol, starch, pregelatinized starch, sucrose, sulfobutylether-β-cyclodextrin, talc, tragacanth, trehalose, and xylitol.

In some embodiments, the pharmaceutical composition comprises one or more binders. Binders may be used, e.g., to impart cohesive qualities to a tablet or a capsule, and thus ensure that the formulation remains intact after compression. In some embodiments, the binder is selected from acacia gum, agar, alginic acid, calcium carbonate, calcium lactate, carbomers (e.g., acrylic acid polymer, carboxy polymethylene, polyacrylic acid, carboxyvinyl polymer), carboxymethylcellulose sodium, carrageenan, cellulose acetate phthalate, ceratonia, chitosan, copovidone, corn starch, cottonseed oil, dextrates, dextrin, dextrose, ethylcellulose, gelatin, glyceryl behenate, guar gum, hydrogenated vegetable oil type I, hydroxyethylcellulose, hydroxyethylmethyl cellulose, hydroxypropyl cellulose, hydroxypropyl methylcellulose, hypromellose, inulin, lactose, magnesium aluminum silicate, maltodextrin, maltose, methylcellulose, microcrystalline cellulose, pectin, poloxamer, polycarbohil, polydextrose, polyethylene oxide, polymetharylates, polyvinylpyrrolidone, pre-gelatinized starch, povidone, sodium alginate, starch, stearic acid, sucrose, tricaprylin, vitamin E polyethylene glycol succinate, and zein.

Suitable binders include, but are not limited to, starch (including potato starch, corn starch, and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone (PVP), cellulosic polymers (including hydroxypropyl cellulose (HPC), hydroxypropylmethylcellulose (HPMC), methyl cellulose, ethyl cellulose, hydroxyethyl cellulose (HEC), carboxymethyl cellulose and the like), veegum, carbomer (e.g., carbopol), sodium, dextrin, guar gum, hydrogenated vegetable oil, magnesium aluminum silicate, maltodextrin, polymethacrylates, povidone (e.g., KOLLIDON, PLASDONE), microcrystalline cellulose, among others. Binding agents also include, e.g., acacia, agar, alginic acid, cabomers, carrageenan, cellulose acetate phthalate, ceratonia, chitosan, confectioner's sugar, copovidone, dextrates, dextrin, dextrose, ethylcellulose, gelatin, glyceryl behenate, guar gum, hydroxyethyl cellulose, hydroxyethylmethyl cellulose, hydroxypropyl cellulose, hydroxypropyl starch, hypromellose, inulin, lactose, magnesium aluminum silicate, maltodextrin, maltose, methylcellulose, poloxamer, polycarbophil, polydextrose, polyethylene oxide, polymethylacrylates, povidone, sodium alginate, sodium carboxymethylcellulose, starch, pregelatinized starch, stearic acid, sucrose, and zein.

Suitable forms of microcrystalline cellulose include, but are not limited to, the materials sold as AVICEL-PH-101, AVICEL-PH-103 AVICEL RC-581, AVICEL-PH-105 (FMC Corporation, Marcus Hook, Pa.), and mixtures thereof. In some embodiment, a specific binder is a mixture of microcrystalline cellulose and sodium carboxymethyl cellulose sold as AVICEL RC-581. Suitable anhydrous or low moisture excipients or additives include AVTCEL-PH-103™ and Starch 1500 LM.

In some embodiments, the pharmaceutical composition comprises one or more disintegrants. In certain embodiments, the disintegrant is selected from alginic acid, calcium alginate, carboxymethylcellulose calcium, carboxymethylcellulose sodium, cellulose, chitosan, colloidal silicon dioxide, corn starch, croscarmellose sodium, crospovidone, docusate sodium, glycine, guar gum, hydroxypropyl cellulose, magnesium aluminum silicate, methylcellulose, microcrystalline cellulose, pre-gelatinized starch, polacrilin potassium, povidone, silicates, sodium aliginate, sodium carbonate, and sodium starch glycolate.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof.

In some embodiments, the pharmaceutical composition comprises one or more surfactants. In some embodiments, the surfactant is selected from polyoxyethylene (20) sorbitan monolaurate (e.g., Tween-20), polyoxyethylene (20) sorbitan monooleate (e.g., Tween-80), sodium lauryl sulfate, and sodium dodecyl sulfate.

In some embodiments, the pharmaceutical composition comprises one or more pore formers. In some embodiments, the pore former is selected from hydroxypropylcellulose, hydroxypropylmethylcellulose, polyethyleneglycol, poloaxamer 188, povidone (e.g., Kollidon K25/K30), or sugar (e.g., glucose, mannose, fructose, and sucrose).

In some embodiments, the pharmaceutical composition comprises one or more glidants. In some embodiments, the glidant is selected from calcium phosphate, cellulose, colloidal silicon dioxide, fumed silica, magnesium oxide, magnesium silicate, magnesium stearate, magnesium trisilicate, and talc. Suitable glidants include, but are not limited to, colloidal silicon dioxide, CAB—O-SIL™ (Cabot Co. of Boston, MA), and asbestos-free talc.

In some embodiments, the pharmaceutical composition comprises one or more lubricants. In some embodiments, the lubricant is selected from calcium stearate, glycerin monosterate, glyceryl behenate, glyceryl palmitostearate, hydrogenated castor oil, hydrogenated vegetable oil, light mineral oil, myristic acid, poloxamer, polyethylene glycol, sodium benzoate, sodium chloride, sodium lauryl sulfate, sodium stearyl fumarate, solid polyethylene glycols, stearic acid, and talc.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof.

In some embodiments, the pharmaceutical composition comprises one or more film coating agents. In some embodiments, the film coating comprises a poly(vinyl alcohol) base. In some embodiments, the film coating includes a coloring agent or pigment. In some embodiments, the film coating is Opadry II® such as Opadry II® yellow.

Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

Suitable flavoring agents include, but are not limited to, natural flavors extracted from plants, such as fruits, and synthetic blends of compounds which produce a pleasant taste sensation, such as peppermint and methyl salicylate.

Suitable sweetening agents include, but are not limited to, sucrose, lactose, mannitol, syrups, glycerin, and artificial sweeteners, such as saccharin and aspartame.

Suitable emulsifying agents include, but are not limited to, gelatin, acacia, tragacanth, bentonite, and surfactants, such as polyoxyethylene sorbitan monooleate (Tween-20), polyoxyethylene sorbitan monooleate 80 (Tween-80), and triethanolamine oleate.

Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone.

Suitable preservatives include, but are not limited to, glycerin, methyl and propylparaben, benzoic acid, sodium benzoate, and alcohol.

Suitable wetting agents include, but are not limited to, propylene glycol monostearate, sorbitan monooleate, diethylene glycol monolaurate, and polyoxyethylene lauryl ether.

Suitable solvents include, but are not limited to, glycerin, sorbitol, ethyl alcohol, and syrup.

Suitable non-aqueous liquids utilized in emulsions include, but are not limited to, mineral oil and cottonseed oil.

Suitable organic acids include, but are not limited to, citric and tartaric acid.

Suitable sources of carbon dioxide include, but are not limited to, sodium bicarbonate and sodium carbonate.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

A tablet dosage form can be prepared from the active ingredient in powdered, crystalline, or granular forms, alone or in combination with one or more carriers or excipients described herein, including binders, disintegrants, controlled-release polymers, lubricants, diluents, and/or colorants.

A tablet of the present disclosure can be formulated for rapid, sustained, extended, or modified release.

In some embodiments, a unit dosage form described herein comprises one or more pharmaceutically acceptable excipients selected from microcrystalline cellulose, lactose monohydrate (modified), croscarmellose sodium, hydroxypropyl cellulose, and magnesium stearate.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compound described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

The active compound can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein can include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated. Additionally, transdermal patches can be used, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, a composition, as described herein, can include a prodrug of Compound 1. The term “prodrug,” as used herein, means a compound that is convertible in vivo by metabolic means (e.g., by hydrolysis) to a compound. Various general forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). Design and Application of Prodrugs, Textbook of Drug Design and Development, Chapter 5, 113-191 (1991), Bundgaard, 9 9=., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is hereby incorporated by reference in its entirety.

For oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrants include, without limitation, starch, methyl cellulose, agar, bentonite, xanthan gum starches, agar, alginic acid or its sodium salt, or effervescent mixtures, croscarmellose or its sodium salt, and the like. Diluents include, e.g., lactose, dextrose, sucrose, mannitol, sorbitol, cellulose and/or glycine.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A therapeutically effective dose, of a compound described herein in an oral formulation, may vary from 0.15 mg/kg to 20 mg/kg patient body weight per day, more particularly 0.015 to 1.0 mg/kg, which can be administered in single or multiple doses per day. For oral administration, the drug can be delivered in the form of tablets or capsules containing 1 mg to 100 mg of the active ingredient specifically, 1 mg, 5 mg, 10 mg, 20 mg, 50 mg, or 100 mg, or in the forms of tables or capsules containing at least 1%, 2%, 5%, 10%, 15%, 20%, 25%, 30%, 40%, 50% (w/w) of the active ingredient. For example, the capsules may contain 50 mg of the active ingredient, or 5-10% (w/w) of the active ingredient. For example, the tablets may contain 100 mg of the active ingredient, or 20-50% (w/w) of the active ingredient. For example, the tablet may contain, in addition to the active ingredient, a disintegrant or emollient (e.g., croscarmellose or its sodium salt and methyl cellulose), a diluent (e.g., microcrystalline cellulose), and a lubricant (e.g., sodium stearate and magnesium stearate). The drug can be administered on a daily basis either once, twice or more per day.

For administration by inhalation, the compounds can be delivered in the form of an aerosol spray from pressured container or dispenser, which contains a suitable propellant, e.g., a gas such as carbon dioxide, or a nebulizer.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Penetration enhancers promote the penetration of drugs through the corneal barrier and change the integrity of the epithelial cell layer. Frequently used penetration enhancers in ocular formulations include cyclodextrin, dimethylsulphoxide (DMSO), ethylenediaminetetraacetic acid (EDTA), sodium glycocholate and related cholates, Tween 20 (a non-ionic polysorbate surfactant), Brij 35 (polyoxyethylene lauryl ether), saponins and bile salts. Generally, penetration enhancers such as EDTA and cholates transiently loosen the tight junctions between adjacent cells of the corneal epithelium. Thus, penetration enhancers, when applied topically to the eye, have been successfully applied to the delivery of protein and peptides through the corneal epithelium. In some embodiments, a formulation described herein includes a penetration enhancer such as polyoxyethylene-9-lauryl ether, sodium deoxycholate, sodium glycocholate, or sodium taurocholate.

In some embodiments, a pharmaceutically acceptable excipient in an ophthalmic solution or transmucosal formulation described herein is a cyclodextrin. Cyclodextrins are known to act as permeation enhancers and mucoadhesive agents. In some embodiments, the cyclodextrin is α-, β- or γ-cyclodextrin. In some embodiments, the cyclodextrin is a pharmaceutically acceptable derivative of a cyclodextrin, including, but not limited to, the hydroxyalkyl derivatives of α-, β- and γ-cyclodextrin (especially the hydroxyethyl and hydroxypropyl derivatives of β-cyclodextrin and γ-cyclodextrin), randomly methylated β-cyclodextrin, sulfobutylether β-cyclodextrin, sulfobutylether γ-cyclodextrin, and the so-called branched β- and γ-cyclodextrin derivatives such as glucosyl-β-cyclodextrin and glucosyl-γ-cyclodextrin. The natural cyclodextrins are either used alone or in a mixture of two or more cyclodextrins, by way of non-limiting example, a mixture of the γ-cyclodextrin and the more water-soluble hydroxypropyl γ-cyclodextrin, or γ-cyclodextrin and sulfobutylether γ-cyclodextrin, or β-cyclodextrin and hydroxypropyl-β-cyclodextrin, or β-cyclodextrin and sulfobutylether β-cyclodextrin.

In some embodiments, a cyclodextrin in an ophthalmic solution or transmucosal formulation described herein is at a concentration of 0 to 20% w/v. In some embodiments, a cyclodextrin in an ophthalmic solution described herein is at a concentration of 1 to 18% w/v, 1 to 16% w/v, 1 to 14% w/v, 2 to 12% w/v, 4 to 10% w/v, 5 to 9% w/v, or 6 to 8% w/v. In some embodiments, the cyclodextrin in an ophthalmic solution described herein is at a concentration of 7% to 11% w/v. In some embodiments, a cyclodextrin in an ophthalmic solution described herein is at a concentration of about 1% w/v, 2% w/v, 3% w/v, 4% w/v, 5% w/v, 6% w/v, 7% w/v, 8% w/v, 9% w/v, 10% w/v, 11% w/v, 12% w/v, 13% w/v, 14% w/v, 15% w/v, 16% w/v, 17% w/v, 18% w/v, 19% w/v, or 20% w/v.

In some embodiments, a pharmaceutically acceptable excipient in an ophthalmic solution or transmucosal formulation described herein is sulfobutylether-β-cyclodextrin, in particular at any of the specified concentrations and ranges of concentrations above, such as about 7% w/v. In some embodiments, a pharmaceutically acceptable excipient in an ophthalmic solution described herein is hydroxypropyl-β-cyclodextrin, in particular at any of the specified concentrations and ranges of concentrations specified above, such as about 7% w/v.

In some embodiments, an ophthalmic solution described herein comprises a pharmaceutically acceptable buffering agent. In some embodiments, a pharmaceutically acceptable buffering agent is a phosphate buffer, citrate buffer, tris buffer, histidine buffer or acetate buffer.

In some embodiments, a pharmaceutically acceptable buffering agent is sodium phosphate, dibasic. In some embodiments, a pharmaceutically acceptable buffering agent is sodium phosphate, monobasic. In some embodiments, a pharmaceutically acceptable buffering agent is a mixture of sodium phosphate, dibasic, and sodium phosphate, monobasic. In some embodiments, an ophthalmic solution described herein comprises about 0.083% w/v sodium phosphate, dibasic, and about 0.017% w/v sodium phosphate, monobasic.

In some embodiments, the ophthalmic solution described herein is at an approximately neutral pH. In some embodiments, an ophthalmic solution described herein is at a pH of 6.5 to 8. In some embodiments, an ophthalmic solution described herein is at a pH of 6.9 to 7.7. In some embodiments, an ophthalmic solution described herein is at a pH of 7.1 to 7.5. In some embodiments, an ophthalmic solution described herein is at a pH of about 7.3.

Pharmaceutically acceptable acids and/or bases may be used in the ophthalmic solution to adjust pH. In some embodiments, an ophthalmic solution described herein comprises a pharmaceutically acceptable acid. In some embodiments, an ophthalmic solution described herein comprises a pharmaceutically acceptable base. In some embodiments, an ophthalmic solution described herein comprises a pharmaceutically acceptable acid and base. In some embodiments, a pharmaceutically acceptable acid is hydrochloric acid. In some embodiments, pharmaceutically acceptable base is sodium hydroxide.

In some embodiments, an ophthalmic solution described herein comprises a tonicity agent. In some embodiments, a tonicity agent is selected from the group consisting of dextrose, potassium chloride, propylene glycol, and sodium chloride. In some embodiments, an ophthalmic solution described herein comprises a tonicity agent at a concentration of less than about 0.5% w/v. In some embodiments, an ophthalmic solution described herein comprises a tonicity agent at a concentration of about 0.45%, 0.4%, 0.35%, 0.3%, 0.25%, 0.2%, 0.15%, or 0.1% w/v. In some embodiments, a tonicity agent is sodium chloride.

Parenteral formulations comprising the compound described herein can be prepared in aqueous isotonic solutions or suspensions, and suppositories are advantageously prepared from fatty emulsions or suspensions. The formulations may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional methods, and may contain about 0.1 to 75%, preferably about 1 to 50%, of a compound described herein.

The phrases “parenteral administration” and “administered parenterally” are art-recognized terms, and include modes of administration other than enteral and topical administration, such as by injection, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

Formulations for topical administration to the skin can include, for example, ointments, creams, gels and pastes comprising the primary amine compound in a pharmaceutical acceptable carrier. The formulation of the primary amine compound for topical use includes the preparation of oleaginous or water-soluble ointment bases, as is well known to those in the art. For example, these formulations may include vegetable oils, animal fats, and, for example, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin and glyceryl monostearate. Various water-soluble ointment bases may also be used, including glycol ethers and derivatives, polyethylene glycols, polyoxyl 40 stearate and polysorbates.

The formulations for topical administration may contain the compound used in the present application at a concentration in the range of 0.001-10%, 0.05-10%, 0.1-10%, 0.2-10%, 0.5-10%, 1-10%, 2-10%, 3-10%, 4-10%, 5-10%, or 7-10% (weight/volume), or in the range of 0.001-2.0%, 0.001-1.5%, or 0.001-1.0%, (weight/volume), or in the range of 0.05-2.0%, 0.05-1.5%, or 0.05-1.0%, (weight/volume), or in the range of 0.1-5.0%, 0.1-2.0%, 0.1-1.5%, or 0.1-1.0% (weight/volume), or in the range of 0.5-5.0%, 0.5-2.0%, 0.5-1.5%, or 0.5-1.0% (weight/volume), or in the range of 1-5.0%, 1-2.0%, or 1-1.5% (weight/volume). The formulations for topical administration may also contain the compound used in the present application at a concentration in the range of 0.001-2.5%, 0.01-2.5%, 0.05-2.0%, 0.1-2.0%, 0.2-2.0%, 0.5-2.0%, or 1-2.0% (weight/weight), or in the range of 0.001-2.0%, 0.001-1.5%, 0.001-1.0%, or 0.001-5% (weight/weight).

In some embodiments, the compound or pharmaceutically acceptable salt thereof is administered systemically. In some embodiments, the compound or pharmaceutically acceptable salt thereof is administered orally as part of a solid pharmaceutical composition. In some embodiments, the pharmaceutical composition is a liquid. In some embodiments, the pharmaceutical composition is administered as a liquid via nasogastric tube.

In some embodiments, the compound or pharmaceutically acceptable salt thereof is administered once, twice, thrice, or four times per day. In some embodiments, the compound or pharmaceutically acceptable salt thereof is administered twice per day. In some embodiments, the dose of the compound or pharmaceutically acceptable salt thereof is between about 1 mg BID (i.e., twice per day) to about 20 mg BID.

In some embodiments, the pharmaceutical composition is administered daily in one or more divided doses. In some embodiments, the composition is administered once per day (qua diem; QD). In some embodiments, the composition is administered twice per day (bis in die; BID). In some embodiments, the composition is administered thrice per day (ter in die; TID). In some embodiments, the composition is administered four times per day (quarter in die; QID). In some embodiments, the composition is administered every four (4) hours (quaque four hours; q4h).

In some embodiments, the solid form of Compound 1 is substantially amorphous or crystalline, or is a mixture thereof. In some embodiments, the solid form is substantially free of impurities.

In certain embodiments, Compound 1 is a crystalline solid. In some embodiments, Compound 1 is a crystalline solid substantially free of amorphous Compound 1. As used herein, the term “substantially free of amorphous Compound 1” means that the compound contains no significant amount of amorphous Compound 1. In some embodiments, at least about 95% by weight of crystalline Compound 1 is present. In still other embodiments described herein, at least about 99% by weight of crystalline Compound 1 is present.

The compound may be formulated as an SDD formulation. “SDD” as used herein, refers to a pharmaceutical formulation (e.g., of Compound 1 or a pharmaceutically acceptable salt thereof) which is a spray dried formulation. In some embodiments, the formulation comprises a compound of the disclosure (e.g., Compound 1 or a pharmaceutically acceptable salt thereof) and hypromellose acetate succinate (HPMCAS). In one embodiment, the HMPCAS is HPMCAS-M, wherein the “M” indicates (acetyl content 7.0% to 11.0%, succinoyl content 10% to 14%). The use of spray-drying to produce powders from fluid feed stocks is well known, with applications ranging from powdered milk to bulk chemicals and pharmaceuticals. See U.S. Pat. No. 4,187,617 and Mujumbar et al., 91 Drying, pages 56-73 (1991). The use of spray-drying to form solid amorphous dispersions of drugs and concentration-enhancing polymers is also known. See European Patent Applications Nos. 0 901 786, 1 027 886, 1 027 887, 1 027 888, and PCT Applications Nos. WO 00/168092 and WO 00/168055, each of which is hereby incorporated by reference. A typical spray-drying apparatus comprises a drying chamber, atomizing means for atomizing a solvent-containing liquid feed into the drying chamber, a source of heated drying gas directed into the drying chamber and dried product collection means for separating the dried product from the cooled drying gas and vaporized solvent stream following its exit from the drying chamber. Examples of such apparatus include Niro Models PSD-1, PSD-2 and PSD-4 (Niro A/S, Soeborg, Denmark).

“TPGS” or “Vitamin E TPGS” as a descriptor for a pharmaceutical formulation for a compound of the disclosure, as used herein, refers to a pharmaceutical formulation (e.g., of Compound 1 or a pharmaceutically acceptable salt thereof) which includes the components of (a) the active compound (e.g., Compound 1 or a pharmaceutically acceptable salt thereof); (b) one or more diluents (e.g., microcrystalline cellulose); (c) one or more solubilizers (e.g., D-α-tocopherol polyethylene glycol succinate [Vitamin E TPGS]); and (d) one or more binders (e.g., povidone). The formulation may be prepared using granulation processes (e.g., wet granulation). “Granulation,” as used herein, refers to a process to produce larger or smaller granules or particles of a substance or mixture of substances. The process also may remove fine granules and improve flowability within the formulation. Both wet granulation and/or dry granulation may be employed. Dry granulation is achieved using only a combination of granules without the need for any liquid thereon. Slugging uses a tablet press to form large tablets that vary in weight due to the poor flowability of the formulation. The slugs created are then put through a granulator to be broken down into granules and then compressed once again for a final granulated product.

All publications, patents, patent applications and other documents cited in this application are hereby incorporated by reference in their entireties for all purposes to the same extent as if each individual publication, patent, patent application or other document were individually indicated to be incorporated by reference for all purposes.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

### Example 1: Dissolution of Compound 1 and Excipient Effects Thereon

The key formulation challenge presented by Compound 1 was poor aqueous solubility. This could possibly limit absorption in vivo. Two anhydrous un-solvated forms of Compound 1 (Form C and Material D) were identified in polymorph screening as well as two variable hydrates (Form A up to three moles of water, Form J 1-2 moles of water) and several solvates. Through competitive slurry studies, Form C was determined to be the most stable anhydrous, un-solvated form. Hydrate Form A and likely Form J as well converted to Form C upon dehydration.

Conversion of anhydrous Form C to hydrate Form A took place in solvent slurries where the water activity, a_{w}, was above ˜0.62-0.82. Conversion of Form C to Form A was not observed in water itself after 1 day, likely due to low solubility. After storage of Form C at 90% RH at room temperature for 11 days, no conversion to Form A was observed. As shown in Table 1 below, the poor solubilities of two polymorphic forms of Compound 1 (Form C and Form A) are observed in various aqueous systems (left column).

TABLE 1
Solubility of Compound 1 polymorphs Form
C and Form A in various aqueous systems

| | Form C | Form A |
|---|---|---|
| Aqueous | solubility | solubility |
| Solubility | (mg/ml) | (mg/ml) |
| 10 mM HCl, pH 2.0 | 0.058 | 0.142 |
| 50 mM Acetate | 0.013 | 0.017 |
| buffer, pH 5.0 | | |
| 50 mM Phosphate | 0.004 | 0.016 |
| buffer, pH 6.8 | | |
| SGF, pH 1.2 | 0.035 | 0.033 |
| (0.1% Triton X100) | | |
| FaSSIF, pH 6.5 | 0.029 | 0.031 |
| FeSSIF, pH 5.0 | 0.041 | 0.082 |

TABLE 2
Solubility of Compound 1 in Aqueous Buffer
Solutions and Biorelevant Media

| | pH at | Solubility |
|---|---|---|
| Initial Buffer pH | 48 Hours | μg/mL |
| 1.21^{a} | 1.19^{a} | 58.1^{a} |
| 2.98^{a} | 3.01^{a} | 18.9^{a} |
| 4.46^{a} | 4.49^{a} | 13.3^{a} |
| 6.81^{a} | 6.75^{a} | 4.1^{a} |
| Unbuffered water | 8.6^{b} | 5.5^{b} |
| FaSSIF (pH 1.64)^{a} | 1.50^{a} | 29.1^{a} |
| FeSSIF (pH 5.03)^{a} | 5.03^{a} | 41.0^{a} |
| FaSSGF (pH 1.42)^{a} | 1.42^{a} | 34.6^{a} |

^{a}Average of two runs
^{b}Average of three runs

### Partition Coefficient

ClogP=1.96.

### Dissociation Constant

Based on a pKa of the protonated form of 1.94, the dissociation constant Kₐ=1.15×10⁻².

### Hygroscopicity

Compound 1 Form C showed limited hygroscopicity, with 1.75% water sorption without hysteresis between 5-95% relative humidity. Form A, a variable hydrate, exhibited significant hygroscopicity, with 4.58% water sorption from 5-95% RH.

### Melting Point

The onset of melting of Compound 1 Form C was 244.5° C. with a peak at 244.8° C. by DSC.

### pKa and pH Values

The pKa of the protonated form of Compound 1 was determined to be 1.94±0.01. The pH of a saturated aqueous solution of Compound 1 was 8.6.

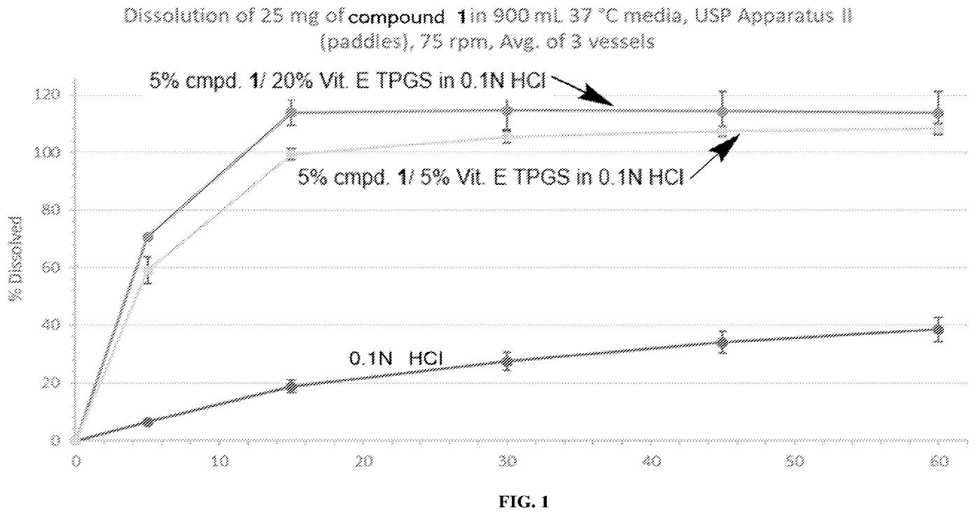

We hypothesized that use of Vitamin E TPGS in a pharmaceutical formation would increase the solubility of Compound 1 and overcome the solubility problem. Dissolution testing of capsules was used as a screening tool to compare different preclinical formulations. FIG. 1 shows the dissolution of the crystalline Compound 1 as Form C blended with microcystalline cellulose (MCC; bottom curve) versus two granulations of Compound 1 as Form C 5% (2^{nd} from bottom or middle curve) or 20% (top curve) of Vitamin E TPGS and MCC. As seen in FIG. 1, the dissolution rate and overall solubility were greatly increased by addition of Vitamin E TPGS.

The TPGS formulation was used in early Phase I clinical studies; the later phase I and current phase 11 clinical trials utilize an alternate SDD formulation described below. Optimization of the SDD formulation included evaluation of several polymers and comparison of drug loading with selected polymers. From the SDD screening exercise, it was determined that a 50:50 ratio of Compound 1 and the polymer HPMC-AS-M (a medium substitution grade of HPMC-AS), provided the best mix of properties including overall spray drying yield, lowest hygroscopicity under high humidity conditions, and best dissolution performance, while balancing the desire to have relatively smaller capsule sizes for clinical application. X-ray powder diffraction confirmed that the SDD process produced an amorphous API dispersion. A human PK assessment of comparative bioavailability of TPGS formulation to SDD formulation was performed. Overall, the exposures were similar or slightly better with a relative % F calculated to be 128%.

As described herein, the SDD formulation containing 50% Compound 1: 50% HPMC-AS-M was successfully developed into size #3 capsules containing 2 mg, 5 mg, 15 mg or 25 mg of Compound 1 and size #2 capsules containing 50 mg of Compound 1. These HPMC-AS-M based SDD formulations are anticipated to provide good exposure of Compound 1 in a clinical setting when orally administered.

Initial toxicology studies utilized a formulation of Compound 1 in 10% Cremophor EL/10% propylene glycol/80% water. However, repeated administration of this formulation to rats demonstrated a “plateau” in systemic exposure such that higher doses did not result in significantly higher exposure. As a result, additional formulations were evaluated in both rats and monkeys including a nanodispersion formulation in 0.5% hydroxypropyl methylcellulose (HPMC) E5 and 0.5% Tween® 80 in deionized water and a spray-dried dispersion (SDD) formulation in 0.5% MC 400 cP and 0.1% Tween® 80 in deionized water. The copolymer used in the initial SDD formulation was polyvinylpyrrolidone/vinyl acetate (PVP-VA) and the drug load was 20%. This spray-dried dispersion formulation showed better solubility when compared to crystalline material and reproducibly demonstrated the highest achievable systemic exposures in animals. This formulation was used for the initial pivotal repeat-dose toxicology testing (through 28-days duration) as well as for in vivo safety pharmacology and genotoxicity studies. Subsequent development of the SDD formulation was conducted to increase the drug load and optimize the characteristics of the formulation for clinical use. These efforts resulted in an SDD formulation using hydroxypropyl methylcellulose acetate succinate (HPMCAS) as the polymer with a drug load of 50%. This improved SDD formulation was used for the longer-term repeat-dose toxicity studies and the developmental toxicity testing in rabbits as well as forming the basis for the clinical formulation.

### Example 2: Development of Oral Capsule Formulations of Compound 1

Compound 1 Drug Product was manufactured in strengths of 5 mg and 25 mg. A description of the drug products are provided in the Table 3.

TABLE 3
Description of Compound 1 Drug Product

| Strength (dosage form) | Description |
|---|---|
| 5 mg | Size 2 hard Swedish orange HPMC capsule |
| (capsule) | containing a white to off-white powder |
| 25 mg | Size 2 hard Swedish orange HPMC capsule |
| (capsule) | containing a white to off-white powder |

### Composition

The composition of Compound 1 Drug Product, 5 mg and 25 mg dosages, are presented in Table 4 and Table 5.

TABLE 4
Unit Composition, Pharmaceutical Function Quality Standards and

| | 5 mg | | 25 mg | | | |
|---|---|---|---|---|---|---|
| | mg per | % | mg per | % | Pharmaceutical | Quality |
| Ingredients | capsule | w/w | capsule | w/w | Function | Standards |
| Active Ingredient | | | | | | |
| Compound 1 | 5 | 10 | 25 | 10 | Active | In house |
| Inactive Ingredients | | | | | | |
| Avicel PH-101 | 34 | 68 | 170 | 68 | Filler | NF |
| Vitamin E TPGS | 10 | 20 | 50 | 20 | Solubility | NF |
| | | | | | Enhancer | |
| PVP K30 | 1 | 2 | 5 | 2 | Binder | NF |
| Total Theoretical | 50 | 100 | 250 | 100 | N/A | N/A |
| Weight | | | | | | |

NF—National Formulary;
N/A—Not applicable

TABLE 5
Total Inactive Ingredient Composition

| | 5 mg | 25 mg | Total Potential Dose |
|---|---|---|---|
| Inactive | Capsule | Capsule | (assuming a 10-capsule maximum) ^{a} |
| Ingredients | mg per unit | mg per unit | mg |
| Avicel PH101 | 34 | 170 | 1700 mg |
| Vitamin E TPGS | 10 | 50 | 500 mg |
| PVP K30 | 1 | 5 | 50 mg |

All excipients listed are generally recognized as safe (GRAS).

### Overages

There are no overages.

### Container Closure

The container closure system for Compound 1 Drug Product, 5 mg and 25 mg, is presented in Table 6 below.

TABLE 6
Drug Product Container Closure System

| | Proposed Packaging | Proposed |
|---|---|---|
| Strength | System | Pack Size |
| 5 mg capsule | 60 cc HDPE bottle with | 12 capsules |
| | induction seal and child | per bottle |
| | resistant closure | |
| 25 mg capsule | 60 cc HDPE bottle with | 12 capsules |
| | induction seal and child | per bottle |
| | resistant closure | |

### Components of the Drug Product

### Drug Substance

Compatibility of the Drug Substance with the Excipients

Excipient compatibility was determined by performing accelerated and real time stability studies of formulated drug in capsules, identical in composition and method of preparation, to the intended clinical dosage forms.

### Physicochemical Characteristics of the Drug Substance

### Physical Properties

The physical properties of Compound 1 are summarized supra. The two main polymorphic forms, A and C, were evaluated during development. Form A is a low melting hydrate, containing up to 3 equivalents of water, that on heating or drying can convert to form C. Form C is the most stable polymorph observed to date and was selected for development. The GLP and GMP batches of drug substance were delivered as form C.

TABLE 7
Compound 1 Physical Properties Summary

| Feature | Form C | Form A |
|---|---|---|
| Solid state description | Crystalline, anhydrous | Crystalline, hydrate |
| Melting point (° C.) | 249.5 | Dehydrates from 40-130° C., |
| | | weak melt at 140° C. |
| Hygroscopicity | Limited hygroscopicity | Significant hygroscopicity |
| | (1.75% wt gain at 75% RH) | (3.0% wt gain at 75% RH) |
| Aqueous Solubility | Lot: a | Lot: b |
| 10 mM HCl, pH 2.0 | 0.058 | 0.142 |
| 50 mM Acetate buffer, pH 5.0 | 0.013 | 0.017 |
| 50 mM Phosphate buffer, pH 6.8 | 0.004 | 0.016 |
| SGF, pH 1.2 (0.1% Triton X100) | 0.035 | 0.033 |
| FaSSIF, pH 6.5 | 0.029 | 0.031 |
| FcSSIF, pH 5.0 | 0.041 | 0.082 |

SGF: simulated gastric fluid, FaSSIF: fasted state simulated intestinal fluid, FcSSIF: fed state simulated intestinal fluid.

### Chemical Properties

pKa: 1.94

### Chemical Stability in Solid State (Crystalline & Amorphous) and in Solution:

Stress testing of Compound 1 Drug Substance was conducted to gain insight into the intrinsic stability of the molecule, to identify any likely degradation products, and to facilitate the development of a stability-indicating analytical method.

### Excipients

MCC (microcrystalline cellulose) was used as an inert filler in the capsule formulations. By adding MCC the volume of fill material is increased which increases the accuracy of volume filling operations. Additionally, because MCC generally has good density and powder flow properties, it aids the capsule filling operations.

PVP K30 (polyvinylpyrrolidone) is used as a binder in the wet granulation process. PVP is a very commonly used binding agent and helps to hold agglomerates of the API and filler (MCC) particles together.

Vitamin E TPGS is a commercially available, non-ionic surfactant, derived from Vitamin E. It is commonly used to enhance the bioavailability of orally administered poorly water-soluble drugs. It was selected based on the high solubility of the drug substance Compound 1 in the excipient.

### Container Closure System

### Description

Compound 1 Drug Product Capsules, 5 mg and 25 mg are packaged in 60 cc HDPE bottles with induction seals and child-resistant caps.

### Choice of Materials

HDPE bottles are the type of container closure system most commonly used for capsule and tablet formulations in the pharmaceutical industry and are readily available. HDPE is chemically inert and provides good protection from moisture and light.

Protection from Moisture

Compound 1 capsules showed no sensitivity to moisture. As a result, the use of a desiccant was not necessary.

Protection from Light

ICH photostability studies demonstrated that Compound 1 is not sensitive to light. Furthermore, the bottles are opaque and will minimize exposure to light during storage of the drug product.

### Safety

Since Compound 1 Drug Product is presented in capsules, the acceptance criteria for microbial limits were established as shown in Table 8. These limits are based on: “Acceptance Criteria for Microbiological Quality of Nonsterile Dosage Forms” specified in USP <1111>“Microbiological Examination of Nonsterile Products: Acceptance Criteria for Pharmaceutical Preparations and Substances for Pharmaceutical Use.”

TABLE 8
Microbiological Testing of Compound 1 Drug Product

| | | Test Results |
|---|---|---|
| | Acceptance | on all Batches |
| Test Item | Criteria | to Date |
| Microbial Limits | Acceptance criteria | Total |
| | for total aerobic | aerobic |
| | microbial count and | microbial |
| | total combined yeast | count: <10³ CFU/g |
| | and mold counts | Total combined |
| | are 10³ CFU/g | yeast and mold |
| | and 10² CFU/g, respectively. | counts: <10² CFU/g |
| | Absence of | Escherichia coli: |
| | Escherichia coli (1 g) | Absent in 1 g |

Microbial limit testing results for release and stability, performed in accordance with USP <61> and <62>, showed the total aerobic microbial count and the total combined yeast and mold counts have not exceeded the acceptance criteria, and no microbial proliferation was observed. Escherichia coli has been absent in all lots tested.

### Manufacturing Process Description

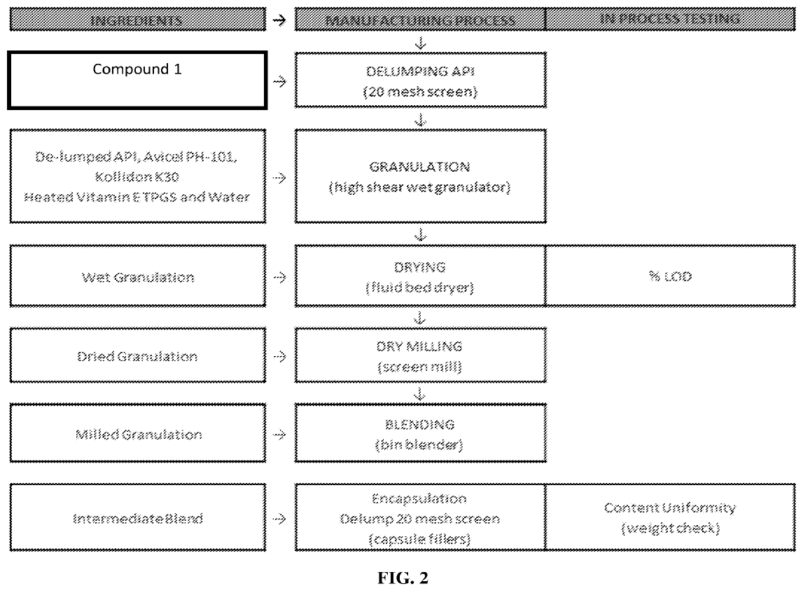

Following the flow diagram shown in FIG. 2 of the disclosure, the formulation process involved wet granulation of Compound 1 with MCC (microcrystalline cellulose, diluent, Avicel PH101), Vitamin E TPGS (solubility enhancer), PVP K30 (polyvinylpyrrolidone, binder), and water. After granulation the wet granules were dried in a fluid bed dryer until LOD≤1.5%, passed through a Comil to break up any aggregates, and blended to provide the bulk capsule fill material. The 5 mg capsules were filled using an Xcelodose system based on weight of input material. The 25 mg capsules were filled used a Profill system based on volume. Content uniformity tests were conducted after capsule filling. Both the 5 mg and 25 mg strength capsules were filled in size #2 hard shell HPMC based capsules. Filled capsules were packaged 12 count each into 60 cc HPDE bottles.

### Description of the in Process Controls

TABLE 9
In Process Controls

| Process Step | In Process Test | Acceptance Criteria |
|---|---|---|
| Granule Drying | % LOD | ≤1.5 LOD |
| Filling | Capsule Uniformity | AV = 15.0 |

### Specifications—Compound 1 Drug Product, Capsule

The quality control specifications for Compound 1 Drug Product Capsules 5 mg and 25 mg are provided in Table 10.

TABLE 10
Quality Control Specifications

| Test | Acceptance Criteria | Test Method |
|---|---|---|
| Appearance of Capsule | Orange hard gelatin capsule | Visual examination |
| | with no holes, cuts, cracks, or | |
| | dents | |
| Appearance of Capsule Contents | White to off-white powder | Visual examination |
| Identification | Retention time of main peak | HPLC-UV |
| | conforms to that of reference | |
| | standard (±3%) | |
| | The UV maxima and minima | |
| | correspond to that of the | |
| | standard (±5 nm) | |
| Label Claim | 90.0%-110.0% | HPLC |
| Potency | 5 mg: 4.5-5.5 mg | HPLC |
| | 25 mg: 22.5-27.5 mg | |
| Dose Uniformity | Meets USP <905> | HPLC |
| | | USP <905> |
| Impurities | Individual ≤0.5% | HPLC |
| | Report RRT and area % of all | |
| | impurities ≥0.05% | |
| | Total ≤3.0% | |
| Water Content | Report results | USP <921> |
| Dissolution | Report results | USP <711> |
| | 15, 30, 45, 60 min | |
| Microbial Limits | TAMC ≤10³ cfu/g | USP <61> |
| | TYMC ≤10² cfu/g | USP <62> |
| | E. coli absent in 1 g | |

### HPLC Method for Identification and Impurities

### Identification

The retention time and UV spectrum of the sample was compared to that of the Compound 1 Drug Substance reference standard. The reversed-phase HPLC method conditions are described in Table 11.

### Impurities

The HPLC method for impurities was based on the drug substance method. The individual related substance peak areas are divided by the total peak area to obtain the percent of each. The total impurities were represented as the summation of each individual impurity. Purity was obtained by subtracting the total impurities from 100%. Table 11 provides a method description.

TABLE 11
Method Conditions for Impurities by HPLC

| Column | Phenomonex Kinetex C18, 2.6 × 100 mm, | | |
|---|---|---|---|
| | 2.6 μm, or equivalent | | |
| Mobile Phase | Mobile Phase A: 0.1% Formic Acid in Water | | |
| | Mobile Phase B: 0.1% Formic Acid in Methanol | | |
| Flow Rate | 1.0 mL/min | | |
| Column Temperature | 40° C. | | |
| Detection (UV) | Identification: 210 to 400 nm | | |
| | 266 nm | | |
| Sample Concentration | 0.2 mg/mL in 10:90 Water:Methanol (v/v) | | |
| Injection Volume | 10 μL | | |
| Total Run Time | 30 min | | |
| Gradient | Time | % Mobile | % Mobile |
| | (min) | Phase A | Phase B |
| | 0.0 | 80 | 20 |
| | 10.0 | 50 | 50 |
| | 25.0 | 20 | 80 |
| | 27.0 | 0 | 100 |
| | 27.1 | 80 | 20 |
| | 30.0 | 80 | 20 |

### Potency, Percent Label Claim, Dosage Uniformity

Potency, percent label claim, and dosage uniformity were determined by HPLC with UV detection. The peak area of the Compound 1 peak was compared to that of the reference standard. Table 12 provides the method conditions for acquiring chromatogram purity. For percent label claim, the potency was compared to that of the nominal value. For dosage uniformity, the acceptance value is calculated in accordance with USP<905>.

TABLE 12
Method Conditions for Determination of Potency, Percent
Label Claim, and Dosage Uniformity by HPLC

| Column | XBridge BEH Shield RP18, 2.1 × 50 mm, | | |
|---|---|---|---|
| | 2.5 μm, or equivalent | | |
| Mobile Phase | Mobile Phase A: 0.1% Formic acid in Water | | |
| | Mobile Phase B: 0.1% Formic acid in Methanol | | |
| Flow Rate | 1.0 mL/min | | |
| Column Temperature | 40° C. | | |
| Detection (UV) | Identification: 210 to 400 nm | | |
| | 343 nm | | |
| Sample Concentration | 0.2 mg/mL in 10:90 Water:Methanol (v/v) | | |
| Injection Volume | 3 μL | | |
| Total Run Time | 8 min | | |
| Gradient | Time | % Mobile | % Mobile |
| | (min) | Phase A | Phase B |
| | 0.0 | 90 | 10 |
| | 1.0 | 90 | 10 |
| | 5.0 | 60 | 40 |
| | 6.0 | 60 | 40 |
| | 6.1 | 90 | 10 |
| | 8.0 | 90 | 10 |

### Dissolution

Dissolution was performed in accordance with USP<711>. USP apparatus II, was used with paddle speed set at 75 rpm in 900 mL of dissolution media at 37° C. After the 60-minute time point, the paddle speed was increased to 200 rpm. Dissolution media was 1% sodium laurel sulfate in 0.1 N HCl. Samples were removed and analyzed by HPLC with UV detection at 343 nm against the reference standard. Chromatographic conditions for the HPLC method are described in Table 13.

TABLE 13
Method Conditions for Determination of Potency, Percent
Label Claim, and Dosage Uniformity by HPLC

| | |
|---|---|
| Column | XBridge BEH Shield RP18, 2.1 × 50 mm, |
| | 2.5 μm, or equivalent |
| Mobile Phase | 75% 0.1% Formic acid in Water |
| | 25% 0.05% Formic acid in Acetonitrile |
| Flow Rate | 1.0 mL/min |
| Column Temperature | 40° C. |
| Detection (UV) | 343 nm |
| Injection Volume | 3 μL |
| Total Run Time | NLT 2x retention time of Compound 1 |
| | (approx. 3 minutes) |
| Gradient | Isocratic |

### Validation of Analytical Procedures—Compound 1 Drug Product, Capsule

The analytical procedures were evaluated to a level appropriate for the current stage of development. The HPLC method for identification and quantification of impurities of Compound 1 Drug Product was qualified for accuracy, precision, linearity, specificity, limits of quantitation and detection, range, peak purity and solution stability. The method for potency, percent label claim, and dosage uniformity has been qualified for accuracy, precision, linearity, specificity, range and solution stability. The method for dissolution was qualified for accuracy, precision, repeatability, linearity, specificity, limits of quantitation and detection, range, filter compatibility, and solution stability.

### Batch Analyses [Drug Product, Capsule]

### Description of Batches

TABLE 14
Batch Analysis Results for Compound 1 Drug Product

| | | Lot No. | | | |
|---|---|---|---|---|---|
| | | 5 | 25 | 158 | 159 |
| | | Strength | | | |
| | | 5 mg | 25 mg | 5 mg | 25 mg |
| | | Batch Size | | | |
| | | Approx. 4000 | Approx. 4000 | Approx. 4464 | Approx. 5587 |
| | | units | units | units | units |
| | | Use | | | |
| | | Supporting | Supporting | | |
| | Proposed | Stability | Stability | Clinical | Clinical |
| Test Method | Specification | Result | | | |
| Appearance of | Orange hard gelatin | Orange | Orange | Orange | Orange |
| Capsule | capsule with no | undamaged | undamaged | capsule, | capsule, defect |
| | holes, cuts, cracks, | capsule | capsule | defect free | free |
| | or dents | | | | |
| Appearance of | White to off-white | White powder | White powder | White powder | White powder |
| Capsule | powder | | | | |
| Contents | | | | | |
| Identification | Retention time of | Not tested | Not tested | Conforms | Conforms |
| | main peak conforms | | | Conforms | Conforms |
| | to that of reference | | | | |
| | standard (±3%) | | | | |
| | The UV maxima | | | | |
| | and minima | | | | |
| | correspond to that | | | | |
| | of the standard (±5 | | | | |
| | nm | | | | |
| Label Claim | 90.0%-110.0% | 96 | 95 | 102.9 | 102.2 |
| Potency | 5 mg: 4.5-5.5 mg | Not reported | Not reported | 5.1 | 25.5 |
| | 25 mg: 22.5-27.5 | | | | |
| | mg | | | | |
| Dose | Meets USP <905> | AV = 12 | AV = 11 | AV = 5 | AV = 13 |
| Uniformity | | | | | |
| Impurities | Individual ≤0.5% | RRT 0.78: 0.02 | RRT 0.78: 0.02 | RRT 1.07: 0.05 | RRT 1.07: 0.05 |
| | Report RRT and | RRT 0.87: 0.04 | RRT 0.87: 0.04 | Total: 0.05 | Total: 0.05 |
| | area % of all | RRT 0.95: 0.02 | RRT 0.95: 0.02 | | |
| | impurities ≥0.05% | RRT 1.07: 0.04 | RRT 1.07: 0.04 | | |
| | Total ≤3.0% | RRT 1.10: 0.06 | RRT 1.10: 0.07 | | |
| | | Total: 0.18 | Total: 0.19 | | |
| Water Content | Report results | 3.19 | 3.76 | 2.22 | 2.36 |
| | (wt %) | | | | |
| Dissolution | Report results (%) | 15 min: 77 | 15 min: 63 | 15 min: 77 | 15 min: 65 |
| | 15, 30, 45, 60 min | 30 min: 86 | 30 min: 84 | 30 min: 100 | 30 min: 94 |
| | | 45 min: 89 | 45 min: 89 | 45 min: 101 | 45 min: 94 |
| | | 60 min: 93 | 60 min: 91 | 60 min: 103 | 60 min: 99 |
| Microbial | TAMC ≤10³ cfu/g | Not tested | Not tested | TAMC <50 | TAMC <50 |
| Limits | TYMC ≤10² cfu/g | | | cfu/g | cfu/g |
| | E. coli absent in 1 g | | | TYMC <50 | TYMC 50 |
| | | | | cfu/g | cfu/g |
| | | | | E. coli absent | E. coli absent |
| | | | | in 1 g | in 1 g |

### Characterization of Impurities

There were no new drug product specific impurities in Compound 1.

### Stability Summary and Conclusion

### Summary of Stability Studies

One month of data is available for the representative non-GMP batches of Compound 1 Drug Product, 5 and 25 mg at both proposed long-term, 2-8° C., and accelerated, 25° C./60% RH and 40° C./75% RH storage conditions.

No significant changes were observed in any of the test parameters.

### Design of Stability Studies

The stability of Compound 1 is represented in Table 15. This protocol was conducted at both proposed long-term, 2-8° C., and accelerated, 25° C./60% RH and 40° C./75% RH storage conditions.

TABLE 15
Stability Protocol for Non-GMP Compound ‘1 Drug Product Lots

| Stability Condition/Stability | | | | | |
|---|---|---|---|---|---|
| Test Time Point (month) | 0 | 1 | 3 | 6 | 12 |
| 2-8° C. | A | A | A | A | A |
| 25° C./60% RH | | A | A | A | A |
| 40° C./75% RH | | A | A | A | — |

A: Appearance, Potency, Impurities, Water Content, Dissolution

The representative stability protocol for the stability of Compound 1 is presented in Table 16. This protocol was designed as a 2-year study at both proposed long-term, 2-8° C., and accelerated, 25° C./60% RH and 40° C./75% RH storage conditions.

TABLE 16
Stability Protocol for GMP Compound 1 Drug

| Stability | | | | | | | | |
|---|---|---|---|---|---|---|---|---|
| Condition/Stability | | | | | | | | |
| Test Time Point | | | | | | | | |
| (month) | 0 | 1 | 3 | 6 | 9 | 12 | 18 | 24 |
| 2-8° C. | A, B | A | A | A | A | A, B | A | A, B |
| 25° C./60% RH | | A | A | A | A | A, B | A | A, B |
| 40° C./75% RH | | A | A | A | — | — | — | — |

A: Appearance, Potency, Impurities, Water Content, Dissolution
B: Microbial Limits
—: No measurement planned

### Batches Tested and Packaging

Table 17 summarizes the lots of Compound 1 Drug Product that were on stability.

TABLE 17
Summary of Compound 1 Drug Product Stability

| | Batch | | Last Time | | |
|---|---|---|---|---|---|
| | Size | Container | Point | Study | |
| (strength) | (units) | Closure | Completed | Duration | Conditions |
| (5 mg) | Approx. | 10 count 60 cc | 1 months | 12 months | 2-8° C. |
| | 4000 | HDPE bottle | | | 25° C./60% RH |
| | | with heat | | | 40° C./75% RH |
| | | induction seal | | | |
| | | and cap | | | |
| (25 mg) | Approx. | 10 count 60 cc | 1 month | 12 months | 2-8° C. |
| | 4000 | HDPE bottle | | | 25° C./60% RH |
| | | with heat | | | 40° C./75% RH |
| | | induction seal | | | |
| | | and cap | | | |
| (5 mg) | Approx. | 12 count 60 cc | None at | 24 months | 2-8° C. |
| | 4464 | HDPE bottle | time of | | 25° C./60% RH |
| | | with heat | filing | | 40° C./75% RH |
| | | induction seal | | | |
| | | and cap | | | |
| (25 mg) | Approx. | 12 count 60 cc | None at | 24 months | 2-8° C. |
| | 5587 | HDPE bottle | time of | | 25° C./60% RH |
| | | with heat | filing | | 40° C./75% RH |
| | | induction seal | | | |
| | | and cap | | | |

### Stability Data

### Results of the Stability Data Under Established Storage and Stress Conditions

The available stability data for the demonstration batches, 5 mg and 25 mg respectively) are described in the tables below.

TABLE 18
Stability Data for Drug Product, 5 mg Stored at 2-8° C.

| | | Storage Time | |
|---|---|---|---|
| Test | Acceptance Criteria | 0 Month | 1 Month |
| Appearance of | Orange hard gelatin capsule | Orange undamaged | Orange undamaged |
| Capsule | with no holes, cuts, cracks, | capsule | capsule |
| | or dents | | |
| Appearance of | White to off-white powder | White powder | White powder |
| Capsule Contents | | | |
| Label Claim | 90.0%-110.0% | 96 | 99 |
| Impurities | Individual ≤0.5% | RRT 0.78: 0.02 | RRT 0.78: ND |
| | Report RRT and area % of | RRT 0.87: 0.04 | RRT 0.87: 0.04 |
| | all impurities ≥0.05% | RRT 0.95: 0.02 | RRT 0.95: ND |
| | Total ≤3.0% | RRT 1.07: 0.04 | RRT 1.07: 0.04 |
| | | RRT 1.10: 0.06 | RRT 1.10: 0.08 |
| | | Total: 0.18 | Total: 0.16 |
| Water Content | Report Results (wt %) | 3.19 | 3.03 |
| Dissolution | Report Results (%) | 15 min: 77 | 15 min: 72 |
| | | 30 min: 86 | 30 min: 86 |
| | | 45 min: 89 | 45 min: 89 |
| | | 60 min: 93 | 60 min: 94 |

ND—Not detected

TABLE 19
Stability Data for Drug Product, 5 mg Stored at 25° C./60% RH

| | | Storage Time | |
|---|---|---|---|
| Test | Acceptance Criteria | 0 Month | 1 Month |
| Appearance of | Orange hard gelatin capsule | Orange undamaged | Orange undamaged |
| Capsule | with no holes, cuts, cracks, | capsule | capsule |
| | or dents | | |
| Appearance of | White to off-white powder | White powder | White powder |
| Capsule Contents | | | |
| Label Claim | 90.0%-110.0% | 96 | 98 |
| Impurities | Individual ≤0.5% | RRT 0.78: 0.02 | RRT 0.78: ND |
| | Report RRT and area % of | RRT 0.87: 0.04 | RRT 0.87: 0.04 |
| | all impurities ≥0.05% | RRT 0.95: 0.02 | RRT 0.95: ND |
| | Total ≤3.0% | RRT 1.07: 0.04 | RRT 1.07: 0.04 |
| | | RRT 1.10: 0.06 | RRT 1.10: 0.08 |
| | | Total: 0.18 | Total: 0.16 |
| Water Content | Report Results (wt %) | 3.19 | 3.33 |
| Dissolution | Report Results (%) | 15 min: 77 | 15 min: 62 |
| | | 30 min: 86 | 30 min: 81 |
| | | 45 min: 89 | 45 min: 89 |
| | | 60 min: 93 | 60 min: 94 |

ND—Not detected

TABLE 20
Stability Data for Drug Product, 5 mg Stored at 40° C./75% RH

| | | Storage Time | |
|---|---|---|---|
| Test | Acceptance Criteria | 0 Month | 1 Month |
| Appearance of | Orange hard gelatin capsule | Orange undamaged | Orange undamaged |
| Capsule | with no holes, cuts, cracks, | capsule | capsule |
| | or dents | | |
| Appearance of | White to off-white powder | White powder | White powder |
| Capsule Contents | | | |
| Label Claim | 90.0%-110.0% | 96 | 98 |
| Impurities | Individual ≤0.5% | RRT 0.78: 0.02 | RRT 0.78: ND |
| | Report RRT and area % of | RRT 0.87: 0.04 | RRT 0.87: 0.04 |
| | all impurities ≥0.05% | RRT 0.95: 0.02 | RRT 0.95: ND |
| | Total ≤3.0% | RRT 1.07: 0.04 | RRT 1.07: 0.04 |
| | | RRT 1.10: 0.06 | RRT 1.10: 0.08 |
| | | Total: 0.18 | Total: 0.16 |
| Water Content | Report Results (wt %) | 3.19 | 3.81 |
| Dissolution | Report Results (%) | 15 min: 77 | 15 min: 71 |
| | | 30 min: 86 | 30 min: 85 |
| | | 45 min: 89 | 45 min: 92 |
| | | 60 min: 93 | 60 min: 96 |

ND—Not detected

TABLE 21
Stability Data for Drug Product, 25 mg Stored at 2-8° C.

| | | Storage Time | |
|---|---|---|---|
| Test | Acceptance Criteria | 0 Month | 1 Month |
| Appearance of | Orange hard gelatin capsule | Orange undamaged | Orange undamaged |
| Capsule | with no holes, cuts, cracks, | capsule | capsule |
| | or dents | | |
| Appearance of | White to off-white powder | White powder | White powder |
| Capsule Contents | | | |
| Label Claim | 90.0%-110.0% | 95 | 96 |
| Impurities | Individual ≤0.5% | RRT 0.78: 0.02 | RRT 0.78: ND |
| | Report RRT and area % of | RRT 0.87: 0.04 | RRT 0.87: 0.04 |
| | all impurities ≥0.05% | RRT 0.95: 0.02 | RRT 0.95: ND |
| | Total ≤3.0% | RRT 1.07: 0.04 | RRT 1.07: 0.04 |
| | | RRT 1.10: 0.07 | RRT 1.10: 0.08 |
| | | Total: 0.19 | Total: 0.16 |
| Water Content | Report Results (wt %) | 3.76 | 2.03 |
| Dissolution | Report Results (%) | 15 min: 63 | 15 min: 51 |
| | | 30 min: 84 | 30 min: 85 |
| | | 45 min: 89 | 45 min: 90 |
| | | 60 min: 91 | 60 min: 95 |

ND—Not detected

TABLE 22
Stability Data for Drug Product,
25 mg Lot Stored at 25° C./60% RH

| | | Storage Time | |
|---|---|---|---|
| Test | Acceptance Criteria | 0 Month | 1 Month |
| Appearance | Orange hard gelatin | Orange | Orange |
| of Capsule | capsule with no holes, | undamaged | undamaged |
| | cuts, cracks, or dents | capsule | capsule |
| Appearance | White to off-white | White powder | White powder |
| of Capsule | powder | | |
| Contents | | | |
| Label Claim | 90.0%-110.0% | 95 | 98 |
| Impurities | Individual ≤ 0.5% | RRT 0.78: 0.02 | RRT 0.78: ND |
| | Report RRT and | RRT 0.87: 0.04 | RRT 0.87: 0.04 |
| | area % of all | RRT 0.95: 0.02 | RRT 0.95: ND |
| | impurities ≥ 0.05% | RRT 1.07: 0.04 | RRT 1.07: 0.04 |
| | Total ≤ 3.0% | RRT 1.10: 0.07 | RRT 1.10: 0.07 |
| | | Total: 0.19 | Total: 0.15 |
| Water Content | Report Results (wt %) | 3.76 | 3.49 |
| Dissolution | Report Results (%) | 15 min: 63 | 15 min: 46 |
| | | 30 min: 84 | 30 min: 81 |
| | | 45 min: 89 | 45 min: 90 |
| | | 60 min: 91 | 60 min: 95 |

ND-Not detected

TABLE 23
Stability Data for Drug Product, 25 mg Stored at 40° C./75% RH

| | | Storage Time | |
|---|---|---|---|
| Test | Acceptance Criteria | 0 Month | 1 Month |
| Appearance | Orange hard gelatin | Orange | Orange |
| of Capsule | capsule with no holes, | undamaged | undamaged |
| | cuts, cracks, or dents | capsule | capsule |
| Appearance | White to off-white | White powder | White powder |
| of Capsule | powder | | |
| Contents | | | |
| Label Claim | 90.0%-110.0% | 95 | 93 |
| Impurities | Individual ≤ 0.5% | RRT 0.78: 0.02 | RRT 0.78: ND |
| | Report RRT and | RRT 0.87: 0.04 | RRT 0.87: 0.04 |
| | area % of all | RRT 0.95: 0.02 | RRT 0.95: ND |
| | impurities ≥ 0.05% | RRT 1.07: 0.04 | RRT 1.07: 0.04 |
| | Total ≤ 3.0% | RRT 1.10: 0.07 | RRT 1.10: 0.08 |
| | | Total: 0.19 | Total: 0.16 |
| Water Content | Report Results (wt %) | 3.76 | 3.53 |
| Dissolution | Report Results (%) | 15 min: 63 | 15 min: 49 |
| | | 30 min: 84 | 30 min: 78 |
| | | 45 min: 89 | 45 min: 84 |
| | | 60 min: 91 | 60 min: 88 |

ND-Not detected

### Example 3: Batch Formulation for Drug Product Capsules IMPD (2 mg Etc)

The following tables provide batch summaries for various dosage forms.

TABLE 24
Batch Formula for Compound 1 SDD Drug Product Capsules, 2 mg
Core Tablet/Capsule Blend

| | | Quantity | Quantity | |
|---|---|---|---|---|
| | Quality | per | per | |
| Component | Standard | Capsule | Batch | Function |
| Spray Dried | In-House | 4.0 | 95.55 | Contains |
| Intermediate* | Standard | | | 50% Active |
| | | | | Ingredient |
| | | | | by weight |

Intragranular Excipients

| | | | | |
|---|---|---|---|---|
| Microcrystalline | NF | 69.3 | 1656.10 | Filler |
| Cellulose | | | | |
| Mannitol | USP | 69.3 | 1656.10 | Filler |
| Croscarmellose | NF | 9.0 | 215.00 | Disintegrant |
| Sodium | | | | |
| Colloidal Silicon | NF | 1.8 | 43.00 | Glidant |
| Dioxide | | | | |
| Sodium Stearyl | NF | 0.9 | 21.50 | Lubricant |
| Fumarate | | | | |

Extragranular Excipients

| | | | | |
|---|---|---|---|---|
| Microcrystalline | NF | 16.2 | 387.00 | Filler |
| Cellulose | | | | |
| Croscarmellose | NF | 9.0 | 215.00 | Disintegrant |
| Sodium | | | | |
| Sodium Stearyl | NF | 0.5 | 10.75 | Lubricant |
| Fumarate | | | | |

| | | |
|---|---|---|
| Total Batch Size | 180.1 | 4300.00 |

*Spray dried intermediate also described as 50:50 Compound 1:HPMC-AS-M SDD containing 50% Compound 1 by weight.
NF - National Formulary;
USP - United States Pharmacopoeia

TABLE 25
Batch Formula for Compound 1 SDD Drug Product Capsules, 5 mg
Core Tablet/Capsule Blend

| | | Quantity | Quantity | Quantity | Quantity | |
|---|---|---|---|---|---|---|
| | | per | per | per | per | |
| | Quality | Capsule | Batch | Capsule | Batch | |
| Component | Standard | (mg) | (g) | (mg) | (g) | Function |
| Spray Dried | In-House | 10 | 26.02 | 10 | 250.0 | Contains 50% |
| Intermediate* | Standard | | | | | Active |
| | | | | | | Ingredient by |
| | | | | | | weight |

Intragranular Excipients

| | | | | | | |
|---|---|---|---|---|---|---|
| Microcrystalline | NF | 65.6 | 170.45 | 66.3 | 1657.98 | Filler |
| Cellulose | | | | | | |
| Mannitol | USP | 65.6 | 170.45 | 66.3 | 1657.98 | Filler |
| Croscarmellose | NF | 9.0 | 23.40 | 9.0 | 224.98 | Disintegrant |
| Sodium | | | | | | |
| Colloidal Silicon | NF | 1.8 | 4.68 | 1.8 | 45.00 | Glidant |
| Dioxide | | | | | | |
| Sodium Lauryl | NF | 1.8 | 4.68 | 0.0 | 0.0 | Wetting Agent |
| Sulfate | | | | | | |
| Sodium Stearyl | NF | 0.9 | 2.34 | 0.9 | 22.50 | Lubricant |
| Fumarate | | | | | | |

Extragranular Excipients

| | | | | | | |
|---|---|---|---|---|---|---|
| Microcrystalline | NF | 16.2 | 42.12 | 16.2 | 404.97 | Filler |
| Cellulose | | | | | | |
| Croscarmellose | NF | 8.7 | 22.70 | 9.0 | 224.98 | Disintegrant |
| Sodium | | | | | | |
| Sodium Stearyl | NF | 0.5 | 1.17 | 0.5 | 11.25 | Lubricant |
| Fumarate | | | | | | |

| | | | | |
|---|---|---|---|---|
| Total Batch Size | 180.1 | 468.01 | 180.1 | 4499.64 |

*Spray dried intermediate also described as 50:50 Compound 1:HPMC-AS-M SDD containing 50% Compound 1 by weight.
NF—National Formulary;
USP—United States Pharmacopoeia

TABLE 26
Batch Formula for Compound 1 SDD Drug Product Capsules, 15 mg
Core Tablet/Capsule Blend

| | Quality | Quantity | Quantity | |
|---|---|---|---|---|
| Component | Standard | per | per Batch | Function |
| Spray Dried | In-House | 30.00 | 2000.16 | Contains |
| Intermediate* | Standard | | | 50% Active |
| | | | | Ingredient |
| | | | | by weight |

Intragranular Excipients

| | | | | |
|---|---|---|---|---|
| Microcrystalline | NF | 56.46 | 3764.32 | Filler |
| Cellulose | | | | |
| Mannitol | USP | 56.46 | 3764.32 | Filler |
| Croscarmellose | NF | 9.0 | 600.04 | Disintegrant |
| Sodium | | | | |
| Colloidal Silicon | NF | 1.8 | 119.96 | Glidant |
| Dioxide | | | | |
| Sodium Stearyl | NF | 0.9 | 60.04 | Lubricant |
| Fumarate | | | | |

Extragranular Excipients

| | | | | |
|---|---|---|---|---|
| Microcrystalline | NF | 5.00 | 333.34 | Filler |
| Cellulose | | | | |
| Croscarmellose | NF | 8.00 | 533.39 | Disintegrant |
| Sodium | | | | |
| Sodium Stearyl | NF | 0.38 | 25.31 | Lubricant |
| Fumarate | | | | |

| | | |
|---|---|---|
| Total Batch Size | 168.00 | 11201.00 |

*Spray dried intermediate also described as 50:50 Compound 1:HPMC-AS-M SDD containing 50% Compound 1 by weight.
NF - National Formulary;
USP - United States Pharmacopoeia

TABLE 27
Batch Formula for Compound 1 SDD Drug Product Capsules, 25 mg
Core Tablet/ Capsule Blend

| | | Quantity | Quantity | Quantity | Quantity | |
|---|---|---|---|---|---|---|
| | | per | per | per | per | |
| | Quality | Capsule | Batch | Capsule | Batch | |
| Component | Standard | (mg) | (g) | (mg) | (g) | Function |
| Spray Dried | In-House | 50 | 114.30 | 50 | 1500.0 | Contains 50% |
| Intermediate* | Standard | | | | | Active |
| | | | | | | Ingredient by |
| | | | | | | weight |

Intragranular Excipients

| | | | | | | |
|---|---|---|---|---|---|---|
| Microcrystalline | NF | 45.6 | 104.14 | 35.9 | 1078.3 | Filler |
| Cellulose | | | | | | |
| Mannitol | USP | 45.6 | 104.14 | 35.9 | 1078.3 | Filler |
| Croscarmellose | NF | 9.0 | 20.57 | 7.5 | 225.00 | Disintegrant |
| Sodium | | | | | | |
| Colloidal | NF | 1.8 | 4.11 | 1.5 | 45.00 | Glidant |
| Silicon Dioxide | | | | | | |
| Sodium Lauryl | NF | 1.8 | 4.11 | 0.0 | 0.0 | Wetting |
| Sulfate | | | | | | Agent |
| Sodium Stearyl | NF | 0.9 | 2.06 | 0.8 | 22.50 | Lubricant |
| Fumarate | | | | | | |

Extragranular Excipients

| | | | | | | |
|---|---|---|---|---|---|---|
| Microcrystalline | NF | 16.2 | 37.03 | 13.5 | 810.0 | Filler |
| Cellulose | | | | | | |
| Croscarmellose | NF | 8.7 | 19.96 | 4.5 | 270.0 | Disintegrant |
| Sodium | | | | | | |
| Sodium Stearyl | NF | 0.5 | 1.03 | 0.4 | 22.6 | Lubricant |
| Fumarate | | | | | | |

| | | | | |
|---|---|---|---|---|
| Total Batch Size | 180.1 | 180.1 | 150.0 | 5051.7 |

*Spray dried intermediate also described as 50:Compound 1:HPMC-AS-M SDD containing 50% Compound 1 by weight.
NF—National Formulary;
USP—United States Pharmacopoeia

TABLE 28
Batch Formula for Compound 1 SDD Drug Product Capsules, 50 mg
Core Tablet/Capsule Blend

| | | Quantity | Quantity | |
|---|---|---|---|---|
| | Quality | per | per | |
| Component | Standard | Capsule | Batch (g) | Function |
| Spray Dried | In-House | 100 | 434.3 | Contains |
| Intermediate* | Standard | | | 50% Active |
| | | | | Ingredient |
| | | | | by weight |

Intragranular Excipients

| | | | | |
|---|---|---|---|---|
| Microcrystalline | NF | 48 | 208.5 | Filler |
| Cellulose | | | | |
| Mannitol | USP | 48 | 208.5 | Filler |
| Croscarmellose | NF | 12.5 | 54.3 | Disintegrant |
| Sodium | | | | |
| Colloidal Silicon | NF | 2.5 | 10.9 | Glidant |
| Dioxide | | | | |
| Sodium Lauryl | NF | 2.5 | 10.9 | Wetting Agent |
| Sulfate | | | | |
| Sodium Stearyl | NF | 1.3 | 5.4 | Lubricant |
| Fumarate | | | | |

Extragranular Excipients

| | | | | |
|---|---|---|---|---|
| Microcrystalline | NF | 22.5 | 97.7 | Filler |
| Cellulose | | | | |
| Croscarmellose | NF | 12.1 | 52.7 | Disintegrant |
| Sodium | | | | |
| Sodium Stearyl | NF | 0.6 | 2.7 | Lubricant |
| Fumarate | | | | |

| | | |
|---|---|---|
| Total Batch Size | 250 | 1085.9 |

*Spray dried intermediate also described as 50:50 Compound 1:HPMC-AS-M SDD containing 50% Compound 1 by weight.
NF - National Formulary;
USP - United States Pharmacopoeia

TABLE 29
Total Inactive Ingredient Composition in Compound 1 SDD Drug Product

| | Dose Form | | | | | | |
|---|---|---|---|---|---|---|---|
| | 2 mg | 5 mg | | 15 mg | 25 mg | | 50 mg |
| | Capsule | Capsule | | Capsule | Capsule | | Capsule |
| | Lot Number | | | | | | |
| | FP- | FP- | FP- | FP- | FP- | FP- | FP- |
| | 20043 | 20009 | 20047 | 20048 | 20010 | 21010 | 20011 |
| Inactive | mg per | mg per | mg per | mg per | mg per | mg per | mg per |
| Ingredients^{a} | unit | unit | unit | unit | unit | unit | unit |
| HPMC-AS-M^{b} | 2.0 | 5.0 | 5.0 | 15.0 | 25.0 | 25.0 | 50.0 |
| MCC | 85.5 | 81.8 | 82.5 | 61.5 | 61.8 | 49.4 | 70.5 |
| Mannitol | 69.3 | 65.6 | 66.3 | 56.5 | 45.6 | 35.9 | 48.0 |
| Croscarmellose | 18.0 | 17.7 | 18.0 | 17.0 | 17.7 | 12.0 | 24.6 |
| Sodium | | | | | | | |
| Silicon | 1.8 | 1.8 | 1.0 | 1.8 | 1.8 | 1.5 | 2.5 |
| Dioxide | | | | | | | |
| Sodium Lauryl | 0.0 | 1.8 | 0.0 | 0.0 | 1.8 | 0.0 | 2.5 |
| Sulfate | | | | | | | |
| Sodium Stearyl | 1.4 | 1.4 | 1.4 | 1.3 | 1.4 | 1.2 | 1.9 |
| Fumarate | | | | | | | |

^{a}The total potential excipient dose is within the human use experience citied in the FDA inactive ingredient database).
^{b}HPMC-AS-M (hydroxypropylmethylcellulose-acetate, succinate, Grade M) is part of the spray dried intermediate which contains 50:50 Compound 1:HPMC-AS-M by weight.

### Description of Manufacturing Process and Process Controls

Following the flow diagram shown in FIG. 4 for manufacturing of Compound 1 SDD Drug Product, the formulation process prepared the SDD by dissolving Compound 1 and HPMC-AS in organic solvent, then passing the liquid through a 100-mesh screen followed by spray drying to produce the SDI (spray dried intermediate), and secondary tray drying to remove residual solvents. An in-process test to confirm residual solvents are below ICH levels is conducted after spray drying.

Once dry, the SDI was blended with intragranular excipients, then delumped with a mill (Comil), blended again, and finally dry granulated with a roller compactor against a 1.00 mm screen, which also mills the ribbons as they are produced. The resulting dry granulate is further blended with extragranular excipients and then filled into capsules using a Profill capsule filling system. The fill weight may be adjusted based on in process control results for potency. Different amounts of intra- and extra-granular excipients are used to produce the blends that will comprise the 2 mg, 5 mg, 15 mg, 25 mg, and 50 mg strength capsules such that the 2 mg, 5 mg, 15 mg and 25 mg blends can fit into size #3 HPMC capsules and the 50 mg blend into size #2 HPMC capsules.

The Compound 1 spray dried intermediate was stored double bagged in HDPE bags with desiccant placed between the bags, and then further enclosed within a heat sealed mylar pouch. The storage conditions are 15-30° C.

To support its long-term storage, Compound 1 SDI was placed on stability and 12 months of data at long-term and 6 months at accelerated conditions are available. The stability protocol was being conducted at both proposed long-term, 25° C./60% RH, and accelerated 40° C./75% RH storage conditions. Available data are provided in Table 30 and Table 31, respectively. No changes under any condition were observed. Stability studies may be extended beyond 24 months to support longer storage of this intermediate.

TABLE 29
Stability Protocol for Non-GMP Compound 1

| Stability | Stability Test Time Point (month) | | | | | | | |
|---|---|---|---|---|---|---|---|---|
| Condition | 0 | 1 | 3 | 6 | 9 | 12 | 18 | 24 |
| 25° C./60% RH | A | A | A | A | A | A | A | A |
| 40° C./75% RH | | A | A | A | — | — | — | |

A: Appearance, Assay, Purity, Water Content, X-Ray Powder Diffraction.

TABLE 30
Stability Data for Compound 1 SDI Lot #FP-19056 Stored at 25° C./60% RH

| | Acceptance | Storage Time | | | | | | |
|---|---|---|---|---|---|---|---|---|
| Test | Criteria | 0 Month | 1 Month | 3 Months | 6 Months | 9 Months | 12 Months | 18 Months |
| Appearance | White to | Off-white | Off-white | Off-white | White | White | White | White |
| | off-white | powder | powder | powder | powder | powder | powder | powder |
| | powder | | | | | | | |
| Assay | Report | 501.2 mg/g | 496.4 mg/g | 496.9 mg/g | 494.3 mg/g | 478.8 mg/g | 496.5 mg/g | 494.9 mg/g |
| | results | | | | | | | |
| Purity | Individual | RRT 1.05: 0.05 | RRT 1.05: 0.05 | RRT 1.05: 0.05 | RRT 1.05: 0.05 | RRT 1.05: 0.05 | RRT 1.05: 0.05 | RRT 1.05: 0.05 |
| | unspecified | Total: 0.05 | Total: 0.05 | Total: 0.05 | Total: 0.05 | Total: 0.05 | Total: 0.05 | Total: 0.05 |
| | impurities: | | | | | | | |
| | Report | | | | | | | |
| | results | | | | | | | |
| | Total | | | | | | | |
| | impurities: | | | | | | | |
| | Report | | | | | | | |
| | results | | | | | | | |
| Water | Report | 1.2 | 1.0 | 1.1 | 0.9 | 0.7 | 0.8 | 0.9 |
| Content | Results | | | | | | | |
| | (wt %) | | | | | | | |
| X-Ray | Report | Consistent | Consistent | Consistent | Consistent | Consistent | Consistent | Consistent |
| Powder | Results | with | with | with | with | with | with | with |
| Diffraction | | amorphous | amorphous | amorphous | amorphous | amorphous | amorphous | amorphous |
| | | form | form | form | form | form | form | form |

TABLE 31
Stability Data for Compound 1 SDI Lot #FP-19056 Stored at 40° C./75% RH

| | Acceptance | Storage Time | | | |
|---|---|---|---|---|---|
| Test | Criteria | 0 Month | 1 Month | 3 Months | 6 Months |
| Appearance | White to | Off-white | Off-white | Off-white | White powder |
| | off-white powder | powder | powder | powder | |
| Assay | Report results | 501.2 mg/g | 495.9 mg/g | 497.9 mg/g | 495.1 mg/g |
| Purity | Individual | RRT 1.05: 0.05 | RRT 1.05: 0.06 | RRT 1.05: 0.05 | RRT 1.05: 0.05 |
| | unspecified | Total: 0.05 | Total: 0.06 | Total: 0.05 | Total: 0.05 |
| | impurities: | | | | |
| | Report results | | | | |
| | Total impurities: | | | | |
| | Report results | | | | |
| Water | Report Results | 1.2 | 0.9 | 0.9 | 0.8 |
| Content | (wt %) | | | | |
| X-Ray | Report Results | Consistent with | Consistent with | Consistent with | Consistent with |
| Powder | | amorphous form | amorphous form | amorphous form | amorphous form |
| Diffraction | | | | | |

Dosage Uniformity. Assay, and Impurities

Dosage uniformity and Assay were determined by HPLC with UV detection. The peak area of the Compound 1 peak was compared to that of the reference standard. The individual related substance peak areas were divided by the total peak area to obtain the percent of each. The total impurities are represented as the summation of each individual impurity. Purity was obtained by subtracting the total impurities from 100%. Table 32 provides method conditions. In typical batches produced by these methods, the SDD drug product produced chromatograms with extremely high purity of Compound 1 (retention time about 14.57 minutes) and very low levels of impurities.

TABLE 32
Method Conditions for Impurities by
HPLC for Compound 1 SDD Drug Product

| Column | Phenomonex Kinetex C18, 4.6 × 100 mm, | | |
|---|---|---|---|
| | 2.6 μm, or equivalent | | |
| Mobile Phase | Mobile Phase A: 0.1% Formic Acid in Water | | |
| | Mobile Phase B: 0.1% Formic Acid in Acetonitrile | | |
| Flow Rate | 1.0 mL/min | | |
| Column | 40° C. | | |
| Temperature | | | |
| Detection (UV) | Identification: 200 to 400 nm | | |
| | 266 nm | | |
| Sample | 0.25 mg/mL in 4:1 Acetonitrile:Water (v/v) | | |
| Concentration | | | |
| Injection Volume | 10 μL | | |
| Total Run Time | 32 min | | |
| | Time | % Mobile | % Mobile |
| | (min) | Phase A | Phase B |
| Gradient | 0 | 95 | 5 |
| | 15 | 60 | 40 |
| | 20 | 42 | 58 |
| | 22 | 5 | 95 |
| | 27 | 5 | 95 |
| | 27.1 | 95 | 5 |
| | 32 | 95 | 5 |

TABLE 33
Batch Analysis Results for Compound 1 SDD Drug Product

| | | Lot No. | | | | | |
|---|---|---|---|---|---|---|---|
| | | DEV-020-117 | DEV-020-119 | DEV-020-121 | FP-20009 | FP-20010 | FP-20011 |
| | | Strength | | | | | |
| | | 5 mg | 25 mg | 50 mg | 5 mg | 25 mg | 50 mg |
| | | Nominal Batch Size | | | | | |
| | | 1000 units | 700 units | 300 units | 998 units | 708 units | 2540 units |
| | | Use | | | | | |
| | | Supporting | | Supporting | | | |
| Test | Proposed | Stability | Development | Stability | Clinical | Clinical | Clinical |
| Method | Specification | Result | | | | | |
| Appearance | White to off- | Not tested | Not tested | Not tested | Off-white | Off-white | Off-white |
| | white to tan | | | | powder in | powder in | powder in |
| | powder in an | | | | opaque | opaque | opaque |
| | opaque white | | | | white | white | white |
| | capsule | | | | capsule | capsule | capsule |
| Identification | Conform to | Not tested | Not tested | Not tested | Conforms | Conforms | Conforms |
| | reference | | | | | | |
| | standard | | | | | | |
| | retention | | | | | | |
| | time and UV | | | | | | |
| | spectrum | | | | | | |
| Assay | 90.0%-110.0% | 94.5 | 88.2 ^{a} | 99.1 | 99.0 | 101.3 | 99.7 |
| | label claim | | | | | | |
| Dose | Meets USP | AV = 8.3 | AV = 20.2 ^{a} | AV = 9.5 | AV = 8.2 | AV = 11.4 | AV = 6.5 |
| Uniformity | <905> | | | | | | |
| Impurities | Individual ≤0.5% | RRT 1.05: 0.05 | RRT 1.05: 0.05 | RRT 1.05: 0.05 | RRT 1.05: 0.05 | RRT 1.05: 0.06 | RRT 1.05: 0.05 |
| | Report RRT and | Total: 0.05 | Total: 0.05 | Total: 0.05 | Total: 0.05 | Total: 0.06 | Total: 0.05 |
| | area % of all | | | | | | |
| | impurities ≥0.05% | | | | | | |
| | Total ≤3.0% | | | | | | |
| Water | Report results | 2.5 | 2.2 | 2.2 | 3.4 | 2.9 | 2.6 |
| Content | (wt %) | | | | | | |

TABLE 34
Batch Analysis Results for Compound 1 SDD Drug Product

| | | Lot No. | | | |
|---|---|---|---|---|---|
| | | FP-20043 | FP-20047 | FP-20048 | FP-21010 |
| | | Strength | | | |
| | | 2 mg | 5 mg | 15 mg | 25 mg |
| | | Nominal Batch Size | | | |
| | | 23400 | 24500 | 66570 | 28000 |
| | | Use | | | |
| Test | Proposed | Clinical | Clinical | Clinical | Clinical |
| Method | Specification | Result | | | |
| Appearance | White to off-white | Off-white | White to | Off-white | White powder |
| | to tan powder in an | powder in | Off-white to | powder in | in an opaque |
| | opaque white capsule | opaque white | tan powder in | opaque white | white capsule |
| | | capsule | opaque white | capsule | |
| | | | capsule | | |
| Identification | Conform to | Conforms | Conforms | Conforms | Conforms |
| | reference standard | | | | |
| | retention time and | | | | |
| | UV spectrum | | | | |
| Assay | 90.0%-110.0% | 100.4 | 98.8 | 101.0^{a} | 97.7 |
| | label claim | | | | |
| Dose | Meets USP <905> | AV = 3.6 | AV = 4.5 | AV = 7.7 ^{a} | AV = 10.8 |
| Uniformity | | | | | (Stage 2) |
| Impurities | Individual ≤ 0.5% | RRT 1.05: BLQ | Total: <0.05 | RRT 1.05: BLQ | RRT 0.85: 0.05 |
| | Report RRT and | Total: <0.05 | | Total: <0.05 | Total: 0.05 |
| | area % of all | | | | |
| | impurities ≥ 0.05% | | | | |
| | Total ≤ 3.0% | | | | |
| Water | Report results (wt %) | 3.3 | 3.3 | 2.9 | 2.4 |
| Content | | | | | |
| Dissolution | Report results (%) | 15 min: 95 | 15 min: 96 | 15 min: 94 | 15 min: 52 |
| | 15, 30, 45, 60 min | 30 min: 95 | 30 min: 95 | 30 min: 99 | 30 min: 80 |
| | | 60 min: 95 | 60 min: 96 | 60 min: 100 | 60 min: 91 |
| | | 75 min: 96 | 75 min: 96 | 75 min: 100 | |
| Microbial | TAMC ≤ 10³ cfu/g | 10 cfu/g | <50 cfu/g | <10 cfu/g | <100 cfu/g |
| Limits | TYMC ≤ 10² cfu/g | <10 cfu/g | <50 cfu/g | <10 cfu/g | <50 cfu/g |
| | E. coli absent in 1 g | Absent in 1 g | Absent in 1 g | Absent in 1 g | Absent in 1 g |

### Dissolution

Dissolution was performed in accordance with USP<711>. USP apparatus II was used with paddle speed set at 100 rpm in 900 mL of dissolution media at 37° C. After the 60-minute time point, the paddle speed was increased to 250 rpm. Dissolution media was 1% sodium laurel sulfate in 20 mM KH₂PO₄ in water, pH 6.8. Samples were removed and analyzed by HPLC with UV detection at 266 nm against the reference standard. Chromatographic conditions for the HPLC method are described in Table 35 purity was assessed by chromatogram.

TABLE 35
Method Conditions for Determination of Dissolution
by HPLC for Compound 1 SDD Drug Product

| Column | Phenomonex Kinetex C18, 4.6 × 100 mm, | | |
|---|---|---|---|
| | 2.6 μm, or equivalent | | |
| Mobile Phase | Mobile Phase A: 0.1% Formic Acid in Water | | |
| | Mobile Phase B: 0.1% Formic Acid in Acetonitrile | | |
| Flow Rate | 1.0 mL/min | | |
| Column | 40° C. | | |
| Temperature | | | |
| Detection (UV) | 266 nm | | |
| Injection Volume | 10 μL | | |
| Total Run Time | 11 min | | |
| | Time | % Mobile | % Mobile |
| | (min) | Phase A | Phase B |
| Gradient | 0 | 80 | 20 |
| | 8 | 20 | 80 |
| | 8.1 | 80 | 20 |
| | 11 | 80 | 20 |

### Validation of Analytical Procedures

The analytical procedures described herein have been evaluated to a level appropriate for the current stage of development.

The HPLC method for identification, assay, dosage uniformity and quantification of impurities in Compound 1 SDD Drug Product was qualified for accuracy, precision, linearity, specificity, limits of quantitation, range and solution stability. The method for dissolution was qualified for accuracy, precision, linearity, specificity, range and solution stability. The Karl Fischer method for water content has been qualified for accuracy and precision.

### Summary of Stability Studies

For Compound 1 SDD Drug Product, twelve months of data are available for the representative non-GMP batches, 5 and 50 mg, at both proposed long-term, 25° C./60% RH, and six months at the accelerated, 40° C./75% RH, storage conditions.

For Compound 1 GMP SDD Drug Product, eighteen months of data is available for the GMP batches 5, 25, and 50 mg at the proposed long-term, 25° C./60% RH, and six months of data is available for the accelerated, 40° C./75% RH, storage conditions.

To reflect the packaging change to blisters, Compound 1 SDD Drug Product capsules 2 mg, 5 mg, 15 mg, and 25 mg were placed on stability. Six months of data for the 2 mg, 5 mg, and 15 mg strengths are available at both proposed long-term, 25° C./60% RH, and accelerated, 40° C./75% RH, storage conditions.

No significant changes were observed in any of the test parameters. For the capsules in blisters, some variability in individual impurities was observed at 3 months and was monitored at subsequent time-points.

The stability protocol for bottled non-GMP Compound 1 SDD Drug Product was conducted at both proposed long-term, 25° C./60% RH, and accelerated, 40° C./75% RH, storage conditions. This stability program supports the clinical use of Compound 1 SDD Drug Product.

The stability protocol for blister packaged GMP Drug Product is designed as a 3-year study at both proposed long-term, 25° C./60% RH, and accelerated, 40° C./75% RH, storage conditions.

### Batches Tested and Packaging

Table 36 summarizes the lots of SDD Drug Product that are currently on stability.

TABLE 36
Summary of Compound 1 SDD Drug Product Lots on Stability

| Lot Number | | Batch | Container | Last Time Point | | Study | |
|---|---|---|---|---|---|---|---|
| (strength) | | Size (units) | Closure | Completed | | Duration | Conditions |
| (5 | mg) | Approx. 540 | 12 count 60 cc | 12 | months | 12 months | 25° C./60% RH |
| | | | HDPE bottle with | | | | 40° C./75% RH |
| | | | heat induction | | | | |
| | | | seal and cap | | | | |
| (50 | mg) | Approx. 125 | 12 count 60 cc | 12 | months | 12 months | 25° C./60% RH |
| | | | HDPE bottle with | | | | 40° C./75% RH |
| | | | heat induction | | | | |
| | | | seal and cap | | | | |
| (5 | mg) | 998 | 12 count 60 cc | 18 | months | 24 months | 25° C./60% RH |
| | | | HDPE bottle with | | | | 40° C./75% RH |
| | | | heat induction | | | | |
| | | | seal and cap | | | | |
| (25 | mg) | 708 | 12 count 60 cc | 18 | months | 24 months | 25° C./60% RH |
| | | | HDPE bottle with | | | | 40° C./75% RH |
| | | | heat induction | | | | |
| | | | seal and cap | | | | |
| (50 | mg) | 2540 | 12 count 60 cc | 18 | months | 24 months | 25° C./60% RH |
| | | | HDPE bottle with | | | | 40° C./75% RH |
| | | | heat induction | | | | |
| | | | seal and cap | | | | |
| (2 | mg) | 23400 | Blister | 6 | month | 36 months | 25° C./60% RH |
| | | | | | | | 40° C./75% RH |
| (5 | mg) | 24500 | Blister | 6 | month | 36 months | 25° C./60% RH |
| | | | | | | | 40° C./75% RH |
| (15 | mg) | 66570 | Blister | 6 | month | 36 months | 25° C./60% RH |
| | | | | | | | 40° C./75% RH |

| | | | | | | |
|---|---|---|---|---|---|---|
| (25 | mg) | 28000 | Blister | Not yet | 36 months | 25° C./60% RH |

| | |
|---|---|
| available | 40° C./75% RH |

### Shelf Life

The expiration of the SDD Drug Product will be 30 months from date of manufacture based on available stability.

### Stability Data

The available stability data for the SDD Drug Product demonstration, 5 mg and 50 mg, respectively are described in tables below.

The available stability data for the SDD Drug Product GMP batches, 5 mg, 25 mg, and 50 mg, respectively are described in tables below.

The available stability data for the SDD Drug Product GMP batches in blister packaging, 2 mg, 5 mg, and 15 mg, respectively are described in tables below.

The acceptance criteria and analytical methods utilized for the stability testing are the same as the release specification.

TABLE 37
Stability Data for SDD Demonstration Batch Drug Product, 5 mg Stored at 25° C./60% RH

| | Acceptance | Storage Time | | | | | |
|---|---|---|---|---|---|---|---|
| Test | Criteria | 0 Month | 1 Month | 3 Months | 6 Months | 9 Months | 12 Months |
| Appearance | Report | Not | White | White | White | White | White |
| | Results | Performed | powder in | powder in | powder in | powder in | powder in |
| | | | opaque | opaque | opaque | opaque | opaque |
| | | | white | white | white | white | white |
| | | | capsules | capsules | capsules | capsules | capsules |
| Assay | Report | 94.5 | 92.2 | 94.6 | 96.5 | 95.2 | 97.6 |
| | Results | | | | | | |
| | (% Label | | | | | | |
| | Claim) | | | | | | |
| Impurities | Report | RRT 1.05: 0.05 | RRT 1.05: 0.05 | RRT 1.05: 0.05 | RRT 1.05: 0.05 | RRT 1.04: 0.05 | RRT 1.04: 0.10 |
| | Results | Total: 0.05 | Total: 0.05 | RRT 1.34: 0.11 | Total: 0.05 | Total: 0.05 | Total: 0.10 |
| | (area %) | | | Total: 0.16 | | | |
| Water | Report | 2.5 | 2.6 | 2.5 | 2.7 | 2.6 | 2.1 |
| Content | Results | | | | | | |
| | (wt %) | | | | | | |
| Dissolution | Report | 15 min: 80 | 15 min: 91 | 15 min: NR | 15 min: NR | 15 min: NR | 15 min: NR |
| | Results | 30 min: 84 | 30 min: 96 | 30 min: NR | 30 min: NR | 30 min: NR | 30 min: NR |
| | (%) | 45 min: 93 | 45 min: 96 | 45 min: NR | 45 min: NR | 45 min: NR | 45 min: NR |
| | | 60 min: 99 | 60 min: 96 | 60 min: 96 | 60 min: 98 | 60 min: 92 | 60 min: 93 |

NR: Not Reported

TABLE 38
Stability Data for Compound 1 SDD Demonstration, 5 mg Stored at 40° C./75% RH

| | Acceptance | Storage Time | | | |
|---|---|---|---|---|---|
| Test | Criteria | 0 Month | 1 Month | 3 Months | 6 Months |
| Appearance | Report Results | Not Performed | White powder | White powder | White powder |
| | | | in opaque white | in opaque white | in opaque white |
| | | | capsules | capsules | capsules |
| Assay | Report Results | 94.5 | 96.4 | 102.9 | 96.0 |
| | (% Label Claim) | | | | |
| Impurities | Report Results | RRT 1.05: 0.05 | RRT 1.05: 0.05 | RRT 1.05: 0.06 | RRT 1.05: 0.05 |
| | (area %) | Total: 0.05 | Total: 0.05 | RRT 1.34: 0.11 | Total: 0.05 |
| | | | | Total: 0.71 | |
| Water | Report Results | 2.5 | 2.6 | 2.7 | 3.3 |
| Content | (wt %) | | | | |
| Dissolution | Report Results | 15 min: 80 | 15 min: 92 | 15 min: NR | 15 min: NR |
| | (%) | 30 min: 84 | 30 min: 97 | 30 min: NR | 30 min: NR |
| | | 45 min: 93 | 45 min: 98 | 45 min: NR | 45 min: NR |
| | | 60 min: 99 | 60 min: 98 | 60 min: 93 | 60 min: 98 |

NR: Not Reported

TABLE 39
Stability Data for Compound 1 SDD Demonstration Batch Drug Product, 50 mg Stored at 25° C./60% RH

| | Acceptance | Storage Time | | | | | |
|---|---|---|---|---|---|---|---|
| Test | Criteria | 0 Month | 1 Month | 3 Months | 6 Months | 9 Months | 12 Months |
| Appearance | Report | Not | White | White | White | White | White |
| | Results | Performed | powder in | powder in | powder in | powder in | powder in |
| | | | opaque | opaque | opaque | opaque | opaque |
| | | | white | white | white | white | white |
| | | | capsules | capsules | capsules | capsule | capsule |
| Assay | Report | 99.1 | 101.7 | 99.2 | 98.9 | 98.4 | 99.3 |
| | Results | | | | | | |
| | (% Label | | | | | | |
| | Claim) | | | | | | |
| Impurities | Report | RRT 1.05: 0.05 | RRT 1.05: 0.05 | RRT 1.05: 0.05 | RRT 1.05 | RRT 1.04: 0.05 | RRT 1.04: 0.12 |
| | Results | Total: 0.05 | Total: 0.05 | RRT 1.34: 0.09 | Total: 0.05 | Total: 0.05 | Total: 0.12 |
| | (area %) | | | Total: 0.14 | | | |
| Water | Report | 2.2 | 2.1 | 2.1 | 2.3 | 2.3 | 2.0 |
| Content | Results | | | | | | |
| | (wt %) | | | | | | |
| Dissolution | Report | 15 min: 94 | 15 min: 78 | 15 min: NR | 15 min: NR | 15 min: NR | 15 min: NR |
| | Results | 30 min: 86 | 30 min: 97 | 30 min: NR | 30 min: NR | 30 min: NR | 30 min: NR |
| | (%) | 45 min: 92 | 45 min: 100 | 45 min: NR | 45 min: NR | 45 min: NR | 45 min: NR |
| | | 60 min: 95 | 60 min: 100 | 60 min: 98 | 60 min: 95 | 60 min: 91 | 60 min: 95 |

NR: Not Reported

TABLE 40
Stability Data for Compound 1 SDD Demonstration
Batch Drug Product, 50 mg Stored at 40° C./75% RH

| | Acceptance | Storage Time | | | |
|---|---|---|---|---|---|
| Test | Criteria | 0 Month | 1 Month | 3 Months | 6 Months |
| Appearance | Report Results | Not | White powder | White powder | White powder |
| | | Performed | in opaque white | in opaque white | in opaque white |
| | | | capsules | capsules | capsules |
| Assay | Report Results | 99.1 | 97.1 | 100.4 | 98.8 |
| | (% Label Claim) | | | | |
| Impurities | Report Results | RRT 1.05: 0.05 | RRT 1.05: 0.06 | RRT 1.05: 0.05 | RRT 1.05: 0.05 |
| | (area %) | Total: 0.05 | Total: 0.06 | RRT 1.34: 0.11 | Total: 0.05 |
| | | | | Total: 0.16 | |
| Water | Report Results | 2.2 | 2.2 | 2.4 | 3.9 |
| Content | (wt %) | | | | |
| Dissolution | Report Results | 15 min: 64 | 15 min: 84 | 15 min: NR | 15 min: NR |
| | (%) | 30 min: 86 | 30 min: 99 | 30 min: NR | 30 min: NR |
| | | 45 min: 92 | 45 min: 101 | 45 min: NR | 45 min: NR |
| | | 60 min: 95 | 60 min: 102 | 60 min: 99 | 60 min: 93 |

NR: Not Reported

TABLE 41
Stability Data for Compound 1 SDD GMP Drug
Product, 5 mg Stored at 25° C./60% RH

| | Acceptance | Storage Time | | |
|---|---|---|---|---|
| Test | Criteria | 0 Month | 1 Month | 3 Months |
| Appearance | White to | Off-white | Off-white | White |
| | off-white to | powder | powder | powder |
| | tan powder | in an | in an | in an |
| | in an opaque | opaque | opaque | opaque |
| | white | white | white | white |
| | capsule | capsule | capsule | capsule |
| Assay | 90.0-110.0% | 99.0 | 95.9 | 98.2 |
| | Label Claim | | | |
| Impurities | Individual ≤0.5% | RRT 1.05: 0.05 | RRT 1.05: 0.05 | RRT 1.05: 0.05 |
| | Report RRT and | Total: 0.05 | RRT 1.17: 0.05 | Total: 0.05 |
| | area % of all | | Total: 0.10 | |
| | impurities ≥0.05% | | | |
| | Total | | | |
| | Impurities ≤3.0% | | | |
| Water | Report | 3.4 | 2.7 | 2.6 |
| Content | Results | | | |
| | (wt %) | | | |
| Dissolution* | Report | 15 min: 83 | 15 min: 79 | 15 min: 91 |
| | Results | 30 min: 94 | 30 min: 99 | 30 min: 94 |
| | (%) | 45 min: 97 | 45 min: 100 | 45 min: 94 |
| | | 60 min: 98 | 60 min: 101 | 60 min: 99 |
| Microbial | TAMC ≤10³ CFU/g | <50 CFU/g | Not | Not |
| Limits | TYMC ≤10² CFU/g | <50 CFU/g | Scheduled | Scheduled |
| | E. coli Absent/g | Absent in 1 g | | |

| | Storage Time | | | |
|---|---|---|---|---|
| Test | 6 Months | 9 Months | 12 Months* | 18 Months |
| Appearance | White | White | White | White |
| | powder | powder | powder | powder |
| | in an | in an | in an | in an |
| | opaque | opaque | opaque | opaque |
| | white | white | white | white |
| | capsule | capsule | capsule | capsule |
| Assay | 96.6 | 99.8 | 98.1 | 98.0 |
| Impurities | RRT 1.05: 0.05 | RRT 1.05: 0.05 | RRT 1.05: 0.05 | RRT 1.05: 0.05 |
| | Total: 0.05 | Total: 0.05 | Total: 0.05 | Total: 0.05 |
| Water | 2.5 | 2.4 | 2.5 | 2.8 |
| Content | | | | |
| Dissolution* | 15 min: 94 | 15 min: 77 | 15 min: 75 | 15 min: 94 |
| | 30 min: 97 | 30 min: 93 | 30 min: 76 | 30 min: 99 |
| | 45 min: 98 | 45 min: 95 | 60 min: 91 | 60 min: 99 |
| | 60 min: 98 | 60 min: 96 | 75 min: 96 | 75 min: 99 |
| Microbial | Not | Not | <100 CFU/g | Not |
| Limits | Scheduled | Scheduled | <50 CFU/g | Scheduled |
| | | | Absent in 1 g | |

*Dissolution profile was modified prior to the 12-Month timepoint due to lack of utility of 45-minute reading, and also to capture infinity spin data.

TABLE 42
Stability Data for Compound 1 SDD GMP Drug Product, 5 mg Stored at 40° C./75% RH

| | Acceptance | Storage Time | | | |
|---|---|---|---|---|---|
| Test | Criteria | 0 Month | 1 Month | 3 Months | 6 Months |
| Appearance | White to off-white to | Off-white | Off-white | White powder | White powder |
| | tan powder in an | powder in an | powder in an | in an opaque | in an opaque |
| | opaque white capsule | opaque white | opaque white | white capsule | white capsule |
| | | capsule | capsule | | |
| Assay | 90.0-110.0% | 99.0 | 96.7 | 97.9 | 96.6 |
| | Label Claim | | | | |
| Impurities | Individual ≤ 0.5% | RRT 1.05: 0.05 | RRT 1.05: 0.05 | RRT 1.05: 0.05 | RRT 1.05: 0.05 |
| | Report RRT and | Total: 0.05 | RRT 1.17: 0.05 | Total: 0.05 | Total: 0.05 |
| | area % of all | | Total: 0.10 | | |
| | impurities ≥ 0.05% | | | | |
| | Total | | | | |
| | Impurities ≤ 3.0% | | | | |
| Water | Report Results (wt %) | 3.4 | 2.5 | 2.9 | 2.5 |
| Content | | | | | |
| Dissolution | Report Results (%) | 15 min: 83 | 15 min: 98 | 15 min: 72 | 15 min: 82 |
| | | 30 min: 94 | 30 min: 101 | 30 min: 89 | 30 min: 96 |
| | | 45 min: 97 | 45 min: 101 | 45 min: 92 | 45 min: 98 |
| | | 60 min: 98 | 60 min: 102 | 60 min: 96 | 60 min: 99 |
| Microbial | TAMC ≤ 10³ CFU/g | <50 CFU/g | Not Scheduled | Not Scheduled | Not Scheduled |
| Limits | TYMC ≤ 10² CFU/g | <50 CFU/g | | | |
| | E. coli Absent/g | Absent in 1 g | | | |

TABLE 43
Stability Data for Compound 1 SDD GMP
Drug Product, 25 mg at 25° C./60% RH

| | Acceptance | Storage Time | | |
|---|---|---|---|---|
| Test | Criteria | 0 Month | 1 Month | 3 Months |
| Appearance | White to | Off-white | Off-white | Off-white |
| | off-white to | powder | powder | powder |
| | tan powder | in an | in an | in an |
| | in an opaque | opaque | opaque | opaque |
| | white | white | white | white |
| | capsule | capsule | capsule | capsule |
| Assay | 90.0-110.0% | 101.3 | 99.9 | 101.2 |
| | Label Claim | | | |
| Impuritics | Individual ≤0.5% | RRT 1.05: 0.06 | RRT 1.05: 0.07 | RRT 1.05: 0.06 |
| | Report RRT and | Total: 0.06 | Total: 0.07 | Total: 0.06 |
| | area % of all | | | |
| | impurities ≥0.05% | | | |
| | Total | | | |
| | Impurities ≤3.0% | | | |
| Water | Report | 2.9 | 2.2 | 2.4 |
| Content | Results | | | |
| | (wt %) | | | |
| Dissolution* | Report | 15 min: 83 | 15 min: 93 | 15 min: 94 |
| | Results | 30 min: 97 | 30 min: 102 | 30 min: 99 |
| | (%) | 45 min: 99 | 45 min: 104 | 45 min: 102 |
| | | 60 min: 101 | 60 min: 105 | 60 min: 103 |
| Microbial | TAMC ≤10³ CFU/g | <50 CFU/g | Not | Not |
| Limits | TYMC ≤10² CFU/g | <50 CFU/g | Scheduled | Scheduled |
| | E. coli Absent/g | Absent in 1 g | | |

| | Storage Time | | | |
|---|---|---|---|---|
| Test | 6 Months | 9 Months | 12 Months* | 18 Months |
| Appearance | White | White | White | White |
| | powder | powder | powder | powder |
| | in an | in an | in an | in an |
| | opaque | opaque | opaque | opaque |
| | white | white | white | white |
| | capsule | capsule | capsule | capsule |
| Assay | 101.9 | 100.8 | 101.2 | 100.5 |
| Impuritics | RRT 1.04: 0.06 | RRT 1.05: 0.05 | RRT 1.05: 0.05 | RRT 1.05: 0.05 |
| | Total: 0.06 | Total: 0.05 | Total: 0.05 | Total: 0.05 |
| Water | 2.7 | 2.1 | 2.2 | 2.6 |
| Content | | | | |
| Dissolution * | 15 min: 94 | 15 min: 85 | 15 min: 88 | 15 min: 85 |
| | 30 min: 102 | 30 min: 94 | 30 min: 97 | 30 min: 101 |
| | 45 min: 104 | 45 min: 96 | 60 min: 101 | 60 min: 100 |
| | 60 min: 104 | 60 min: 100 | 75 min: 102 | 75 min: 102 |
| Microbial | Not | Not | <100 CFU/g | Not |
| Limits | Scheduled | Scheduled | <50 CFU/g | Scheduled |
| | | | Absent in 1 g | |

*Dissolution profile was modified prior to the 12-Month timepoint due to lack of utility of 45 minute reading, and also to capture infinity spin data.

TABLE 44
Stability Data for Compound 1 SDD GMP Drug Product, 25 mg Stored at 40° C./75% RH

| | Acceptance | Storage Time | | | |
|---|---|---|---|---|---|
| Test | Criteria | 0 Month | 1 Month | 3 Months | 6 Months |
| Appearance | White to off-white to | Off-white | Off-white | Off-white | White powder |
| | tan powder in an | powder in an | powder in an | powder in an | in an opaque |
| | opaque white capsule | opaque white | opaque white | opaque white | white capsule |
| | | capsule | capsule | capsule | |
| Assay | 90.0-110.0% | 101.3 | 101.7 | 100.7 | 101.2 |
| | Label Claim | | | | |
| Impurities | Individual ≤ 0.5% | RRT 1.05: 0.06 | RRT 1.05: 0.05 | RRT 1.05: 0.05 | RRT 1.04: 0.05 |
| | Report RRT and | Total: 0.06 | Total: 0.05 | Total: 0.05 | RRT 1.53: 0.05 |
| | area % of all | | | | Total: 0.10 |
| | impurities ≥ 0.05% | | | | |
| | Total | | | | |
| | Impurities ≤ 3.0% | | | | |
| Water | Report Results (wt %) | 2.9 | 2.2 | 2.7 | 3.1 |
| Content | | | | | |
| Dissolution | Report Results (%) | 15 min: 83 | 15 min: 85 | 15 min: 91 | 15 min: 92 |
| | | 30 min: 97 | 30 min: 96 | 30 min: 101 | 30 min: 101 |
| | | 45 min: 99 | 45 min: 101 | 45 min: 103 | 45 min: 103 |
| | | 60 min: 101 | 60 min: 102 | 60 min: 105 | 60 min: 104 |
| Microbial | TAMC ≤ 10³ CFU/g | <50 CFU/g | Not Scheduled | Not Scheduled | Not Scheduled |
| Limits | TYMC ≤ 10² CFU/g | <50 CFU/g | | | |
| | E. coli Absent/g | Absent in 1 g | | | |

TABLE 45
Stability Data for Compound 1 SDD GMP Drug
Product, 50 mg Stored at 25 ° C./60% RH

| | Acceptance | Storage Time | | |
|---|---|---|---|---|
| Test | Criteria | 0 Month | 1 Month | 3 Months |
| Appearance | White to | Off-white | Off-white | Off-white |
| | off-white to | powder | powder | powder |
| | tan powder | in an | in an | in an |
| | in an opaque | opaque | opaque | opaque |
| | white | white | white | white |
| | capsule | capsule | capsule | capsule |
| Assay | 90.0-110.0% | 99.6 | 100.6 | 98.8 |
| | Label Claim | | | |
| Impurities | Individual ≤0.5% | RRT 1.05: 0.05 | RRT 1.05: 0.06 | RRT 1.05: 0.07 |
| | Report RRT and | Total: 0.05 | Total: 0.06 | Total: 0.07 |
| | area % of all | | | |
| | impurities ≥0.05% | | | |
| | Total | | | |
| | Impurities ≤3.0% | | | |
| Water | Report | 2.6 | 2.1 | 2.5 |
| Content | Results | | | |
| | (wt %) | | | |
| Dissolution* | Report | 15 min: 72 | 15 min: 72 | 15 min: 72 |
| | Results | 30 min: 93 | 30 min: 96 | 30 min: 94 |
| | (%) | 45 min: 98 | 45 min: 103 | 45 min: 100 |
| | | 60 min: 101 | 60 min: 104 | 60 min: 103 |
| Microbial | TAMC ≤10³ CFU/g | <50 CFU/g | Not | Not |
| Limits | TYMC ≤10² CFU/g | <50 CFU/g | scheduled | scheduled |
| | E. coli Absent/g | Absent in 1 g | | |

| | Storage Time | | | |
|---|---|---|---|---|
| Test | 6 Months | 9 Months | 12 Months* | 18 Months |
| Appearance | White | White | White | White |
| | powder | powder | powder | powder |
| | in an | in an | in an | in an |
| | opaque | opaque | opaque | opaque |
| | white | white | white | white |
| | capsule | capsule | capsule | capsule |
| Assay | 100.6 | 103.0 | 101.8 | 100.6 |
| Impurities | RRT 1.04: 0.06 | RRT 1.05: 0.05 | RRT 1.05: 0.05 | RRT 1.05: 0.05 |
| | RRT 1.53: 0.06 | Total: 0.05 | Total: 0.05 | Total: 0.05 |
| | Total: 0.11 | | | |
| Water | 2.1 | 2.0 | 2.2 | 2.5 |
| Content | | | | |
| Dissolution* | 15 min: 83 | 15 min: 80 | 15 min: 76 | 15 min: 84 |
| | 30 min: 99 | 30 min: 96 | 30 min: 95 | 30 min: 99 |
| | 45 min: 102 | 45 min: 100 | 60 min: 99 | 60 min: 102 |
| | 60 min: 102 | 60 min: 100 | 75 min: 100 | 75 min: 103 |
| Microbial | Not | Not | <100 CFU/g | Not |
| Limits | scheduled | scheduled | <50 CFU/g | scheduled |
| | | | Absent in 1 g | |

*Dissolution profile was modified prior to the 12-Month timepoint due to lack of utility of 45 minute reading, and also to capture infinity spin data.

TABLE 46
Stability Data for Compound 1 SDD GMP Drug Product, 50 mg Stored at 40° C./75% RH

| | Acceptance | Storage Time | | | |
|---|---|---|---|---|---|
| Test | Criteria | 0 Month | 1 Month | 3 Months | 6 Months |
| Appearance | White to off-white to | Off-white | Off-white | Off-white | Off-white |
| | tan powder in an | powder in an | powder in an | powder in an | powder in an |
| | opaque white capsule | opaque white | opaque white | opaque white | opaque white |
| | | capsule | capsule | capsule | capsule |
| Assay | 90.0-110.0% | 99.6 | 102.6 | 101.4 | 103.5 |
| | Label Claim | | | | |
| Impurities | Individual ≤ 0.5% | RRT 1.05: 0.05 | RRT 1.05: 0.05 | RRT 1.05: 0.05 | RRT 1.04: 0.06 |
| | Report RRT and | Total: 0.05 | Total: 0.05 | Total: 0.05 | Total: 0.06 |
| | area % of all | | | | |
| | impurities ≥ 0.05% | | | | |
| | Total | | | | |
| | Impurities ≤ 3.0% | | | | |
| Water | Report Results (wt %) | 2.6 | 2.1 | 3.5 | 3.5 |
| Content | | | | | |
| Dissolution | Report Results (%) | 15 min: 72 | 15 min: 77 | 15 min: 62 | 15 min: 62 |
| | | 30 min: 93 | 30 min: 9 | 30 min: 90 | 30 min: 90 |
| | | 45 min: 98 | 45 min: 103 | 45 min: 99 | 45 min: 99 |
| | | 60 min: 101 | 60 min: 104 | 60 min: 103 | 60 min: 103 |
| Microbial | TAMC ≤ 10³ CFU/g | <50 CFU/g | Not Scheduled | Not Scheduled | Not Scheduled |
| Limits | TYMC ≤ 10² CFU/g | <50 CFU/g | | | |
| | E. coli Absent/g | Absent in 1 g | | | |

TABLE 47
Stability Data for Compound 1 SDD GMP Drug Product, 2 mg Stored at 25° C./60% RH

| | Acceptance | Storage Time | | | |
|---|---|---|---|---|---|
| Test | Criteria | 0 Month | 1 Month | 3 Months | 6 Months |
| Appearance | White to off-white | White powder | White powder | White powder | White powder |
| | to tan powder in | in an opaque | in an opaque | in an opaque | in an opaque |
| | an opaque white | white capsule | white capsule | white capsule | white capsule |
| | capsule | | | | |
| Assay | 90.0-110.0% | 100.4 | 101.9 | 98.7 | 102.1 |
| | Label Claim | | | | |
| Impurities | Individual ≤0.5% | RRT: 0.85a: <0.05 | RRT 0.85: 0.05 | RRT 0.40: 0.14 | RRT 0.85: <0.05 |
| | Report RRT and | RRT 0.85b: <0.05 | RRT 1.05: <0.05 | RRT 0.85: <0.05 | RRT 1.05: <0.05 |
| | area % of all | RRT 1.05: <0.05 | Total: 0.05 | RRT 1.05: 0.08 | RRT 1.15: <0.05 |
| | impurities ≥0.05% | Total: <0.05 | | RRT 1.34: <0.05 | Total: <0.05 |
| | Total Impurities ≤3.0% | | | Total: 0.23 | |
| Water | Report Results | 3.3 | 3.2 | 3.1 | 3.5 |
| Content | (wt %) | | | | |
| Dissolution | Report Results | 15 min: 95 | 15 min: 94 | 15 min: 93 | 15 min: 97 |
| | (%) | 30 min: 95 | 30 min: 92 | 30 min: 93 | 30 min: 97 |
| | | 60 min: 95 | 60 min: 95 | 60 min: 94 | 60 min: 97 |
| | | 75 min: 96 | 75 min: 96 | 75 min: 94 | 75 min: 97 |
| Microbial | TAMC ≤10³ | <10 CFU/g | Not Scheduled | Not Scheduled | Not Scheduled |
| Limits | CFU/g | <10 CFU/g | | | |
| | TYMC ≤10² | Absent in 1 g | | | |
| | CFU/g | | | | |
| | E. coli Absent/g | | | | |

TABLE 48
Stability Data for Compound 1 SDD GMP Drug Product, 2 mg Stored at 40° C./75% RH

| | Acceptance | Storage Time | | | |
|---|---|---|---|---|---|
| Test | Criteria | 0 Month | 1 Month | 3 Months | 6 Months |
| Appearance | White to off-white | White powder | White powder | White powder | White powder |
| | to tan powder in | in an opaque | in an opaque | in an opaque | in an opaque |
| | an opaque white | white capsule | white capsule | white capsule | white capsule |
| | capsule | | | | |
| Assay | 90.0-110.0% | 100.4 | 102.1 | 95.2 | 98.7 |
| | Label Claim | | | | |
| Impurities | Individual ≤0.5% | RRT 0.85a: <0.05 | RRT 0.85: 0.05 | RRT 0.84: <0.05 | RRT 0.85: <0.05 |
| | Report RRT and | RRT 0.85b: <0.05 | RRT 1.05: <0.05 | RRT 1.05: 0.10 | RRT 0.89: <0.05 |
| | area % of all | RRT 1.05: <0.05 | Total: 0.05 | RRT 1.15: <0.05 | RRT 1.05: <0.05 |
| | impurities ≥0.05% | Total: <0.05 | | Total: 0.10 | RRT 1.15: 0.05 |
| | Total Impurities ≤3.0% | | | | Total 0.05 |
| Water | Report Results | 3.3 | 3.4 | 3.7 | 4.1 |
| Content | (wt %) | | | | |
| Dissolution | Report Results | 15 min: 95 | 15 min: 92 | 15 min: 95 | 15 min: 94 |
| | (%) | 30 min: 95 | 30 min: 89 | 30 min: 95 | 30 min: 95 |
| | | 60 min: 95 | 60 min: 92 | 60 min: 96 | 60 min: 95 |
| | | 75 min: 96 | 75 min: 94 | 75 min: 96 | 75 min: 95 |
| Microbial | TAMC ≤10³ | <10 CFU/g | Not Scheduled | Not Scheduled | Not Scheduled |
| Limits | CFU/g | <10 CFU/g | | | |
| | TYMC ≤10² | Absent in 1 g | | | |
| | CFU/g | | | | |
| | E. coli Absent/g | | | | |

TABLE 49
Stability Data for Compound 1 SDD Demonstration Batch Drug Product, 5 mg Stored at 25° C./60% RH

| | Acceptance | Storage Time | | | | | |
|---|---|---|---|---|---|---|---|
| Test | Criteria | 0 Month | 1 Month | 3 Months | 6 Months | 9 Months | 12 Months |
| Appearance | Report | Not | White | White | White | White | White |
| | Results | Performed | powder in | powder in | powder in | powder in | powder in |
| | | | opaque | opaque | opaque | opaque | opaque |
| | | | white | white | white | white | white |
| | | | capsules | capsules | capsules | capsules | capsules |
| Assay | Report | 94.5 | 92.2 | 94.6 | 96.5 | 95.2 | 97.6 |
| | Results | | | | | | |
| | (% Label | | | | | | |
| | Claim) | | | | | | |
| Impurities | Report | RRT 1.05: 0.05 | RRT 1.05: 0.05 | RRT 1.05: 0.05 | RRT 1.05: 0.05 | RRT 1.04: 0.05 | RRT 1.04: 0.10 |
| | Results | Total: 0.05 | Total: 0.05 | RRT 1.34: 0.11 | Total: 0.05 | Total: 0.05 | Total: 0.10 |
| | (area %) | | | Total: 0.16 | | | |
| Water | Report | 2.5 | 2.6 | 2.5 | 2.7 | 2.6 | 2.1 |
| Content | Results | | | | | | |
| | (wt %) | | | | | | |
| Dissolution | Report | 15 min: 80 | 15 min: 91 | 15 min: NR | 15 min: NR | 15 min: NR | 15 min: NR |
| | Results | 30 min: 84 | 30 min: 96 | 30 min: NR | 30 min: NR | 30 min: NR | 30 min: NR |
| | (%) | 45 min: 93 | 45 min: 96 | 45 min: NR | 45 min: NR | 45 min: NR | 45 min: NR |
| | | 60 min: 99 | 60 min: 96 | 60 min: 96 | 60 min: 98 | 60 min: 92 | 60 min: 93 |

NR: Not Reported

TABLE 50
Stability Data for Compound 1 SDD Demonstration
Batch Drug Product, 5 mg Stored at 40° C./75% RH

| | Acceptance | Storage Time | | | |
|---|---|---|---|---|---|
| Test | Criteria | 0 Month | 1 Month | 3 Months | 6 Months |
| Appearance | Report Results | Not Performed | White powder | White powder | White powder |
| | | | in opaque white | in opaque white | in opaque white |
| | | | capsules | capsules | capsules |
| Assay | Report Results (% | 94.5 | 96.4 | 102.9 | 96.0 |
| | Label Claim) | | | | |
| Impurities | Report Results | RRT 1.05: 0.05 | RRT 1.05: 0.05 | RRT 1.05: 0.06 | RRT 1.05: 0.05 |
| | (area %) | Total: 0.05 | Total: 0.05 | RRT 1.34: 0.11 | Total: 0.05 |
| | | | | Total: 0.71 | |
| Water | Report Results | 2.5 | 2.6 | 2.7 | 3.3 |
| Content | (wt %) | | | | |
| Dissolution | Report Results | 15 min: 80 | 15 min: 92 | 15 min: NR | 15 min: NR |
| | (%) | 30 min: 84 | 30 min: 97 | 30 min: NR | 30 min: NR |
| | | 45 min: 93 | 45 min: 98 | 45 min: NR | 45 min: NR |
| | | 60 min: 99 | 60 min: 98 | 60 min: 93 | 60 min: 98 |

NR: Not Reported

TABLE 51
Stability Data for Compound 1 SDD Demonstration Batch Drug Product, 50 mg Stored at 25° C./60% RH

| | Acceptance | Storage Time | | | | | |
|---|---|---|---|---|---|---|---|
| Test | Criteria | 0 Month | 1 Month | 3 Months | 6 Months | 9 Months | 12 Months |
| Appearance | Report | Not | White | White | White | White | White |
| | Results | Performed | powder in | powder in | powder in | powder in | powder in |
| | | | opaque | opaque | opaque | opaque | opaque |
| | | | white | white | white | white | white |
| | | | capsules | capsules | capsules | capsule | capsule |
| Assay | Report | 99.1 | 101.7 | 99.2 | 98.9 | 98.4 | 99.3 |
| | Results | | | | | | |
| | (% Label | | | | | | |
| | Claim) | | | | | | |
| Impurities | Report | RRT 1.05: 0.05 | RRT 1.05: 0.05 | RRT 1.05: 0.05 | RRT 1.05 | RRT 1.04: 0.05 | RRT 1.04: 0.12 |
| | Results | Total: 0.05 | Total: 0.05 | RRT 1.34: 0.09 | Total: 0.05 | Total: 0.05 | Total: 0.12 |
| | (area %) | | | Total: 0.14 | | | |
| Water | Report | 2.2 | 2.1 | 2.1 | 2.3 | 2.3 | 2.0 |
| Content | Results | | | | | | |
| | (wt %) | | | | | | |
| Dissolution | Report | 15 min: 94 | 15 min: 78 | 15 min: NR | 15 min: NR | 15 min: NR | 15 min: NR |
| | Results | 30 min: 86 | 30 min: 97 | 30 min: NR | 30 min: NR | 30 min: NR | 30 min: NR |
| | (%) | 45 min: 92 | 45 min: 100 | 45 min: NR | 45 min: NR | 45 min: NR | 45 min: NR |
| | | 60 min: 95 | 60 min: 100 | 60 min: 98 | 60 min: 95 | 60 min: 91 | 60 min: 95 |

NR: Not Reported

TABLE 52
Stability Data for Compound 1 SDD Demonstration Batch Drug Product, 50 mg at 40° C./75% RH

| | Acceptance | Storage Time | | | |
|---|---|---|---|---|---|
| Test | Criteria | 0 Month | 1 Month | 3 Months | 6 Months |
| Appearance | Report Results | Not | White powder | White powder | White powder |
| | | Performed | in opaque white | in opaque white | in opaque white |
| | | | capsules | capsules | capsules |
| Assay | Report Results (% | 99.1 | 97.1 | 100.4 | 98.8 |
| | Label Claim) | | | | |
| Impurities | Report Results | RRT 1.05: 0.05 | RRT 1.05: 0.06 | RRT 1.05: 0.05 | RRT 1.05: 0.05 |
| | (area %) | Total: 0.05 | Total: 0.06 | RRT 1.34: 0.11 | Total: 0.05 |
| | | | | Total: 0.16 | |
| Water | Report Results | 2.2 | 2.2 | 2.4 | 3.9 |
| Content | (wt %) | | | | |
| Dissolution | Report Results | 15 min: 64 | 15 min: 84 | 15 min: NR | 15 min: NR |
| | (%) | 30 min: 86 | 30 min: 99 | 30 min: NR | 30 min: NR |
| | | 45 min: 92 | 45 min: 101 | 45 min: NR | 45 min: NR |
| | | 60 min: 95 | 60 min: 102 | 60 min: 99 | 60 min: 93 |

NR: Not Reported

TABLE 53
Stability Data for Compound 1 SDD GMP Drug Product, 5 mg at 25° C./60% RH

| | Acceptance | Storage Time | | |
|---|---|---|---|---|
| Test | Criteria | 0 Month | 1 Month | 3 Months |
| Appearance | White to | Off-white | Off-white | White |
| | off-white to | powder | powder | powder |
| | tan powder | in an | in an | in an |
| | in an opaque | opaque | opaque | opaque |
| | white | white | white | white |
| | capsule | capsule | capsule | capsule |
| Assay | 90.0-110.0% | 99.0 | 95.9 | 98.2 |
| | Label Claim | | | |
| Impurities | Individual ≤0.5% | RRT 1.05: 0.05 | RRT 1.05: 0.05 | RRT 1.05: 0.05 |
| | Report RRT and | Total: 0.05 | RRT 1.17: 0.05 | Total: 0.05 |
| | area % of all | | Total: 0.10 | |
| | impurities ≥0.05% | | | |
| | Total | | | |
| | Impurities ≤3.0% | | | |
| Water | Report | 3.4 | 2.7 | 2.6 |
| Content | Results | | | |
| | (wt %) | | | |
| Dissolution* | Report | 15 min: 83 | 15 min: 79 | 15 min: 91 |
| | Results | 30 min: 94 | 30 min: 99 | 30 min: 94 |
| | (%) | 45 min: 97 | 45 min: 100 | 45 min: 94 |
| | | 60 min: 98 | 60 min: 101 | 60 min: 99 |
| Microbial | TAMC ≤10³ CFU/g | <50 CFU/g | Not | Not |
| Limits | TYMC ≤10² CFU/g | <50 CFU/g | Scheduled | Scheduled |
| | E. coli Absent/g | Absent in 1 g | | |

| | Storage Time | | | |
|---|---|---|---|---|
| Test | 6 Months | 9 Months | 12 Months | 18 Months |
| Appearance | White | White | White | White |
| | powder | powder | powder | powder |
| | in an | in an | in an | in an |
| | opaque | opaque | opaque | opaque |
| | white | white | white | white |
| | capsule | capsule | capsule | capsule |
| Assay | 96.6 | 99.8 | 98.1 | 98.0 |
| Impurities | RRT 1.05: 0.05 | RRT 1.05: 0.05 | RRT 1.05: 0.05 | RRT 1.05: 0.05 |
| | Total: 0.05 | Total: 0.05 | Total: 0.05 | Total: 0.05 |
| Water | 2.5 | 2.4 | 2.5 | 2.8 |
| Content | | | | |
| Dissolution* | 15 min: 94 | 15 min: 77 | 15 min: 75 | 15 min: 94 |
| | 30 min: 97 | 30 min: 93 | 30 min: 76 | 30 min: 99 |
| | 45 min: 98 | 45 min: 95 | 60 min: 91 | 60 min: 99 |
| | 60 min: 98 | 60 min: 96 | 75 min: 96 | 75 min: 99 |
| Microbial | Not | Not | <100 CFU/g | Not |
| Limits | Scheduled | Scheduled | <50 CFU/g | Scheduled |
| | | | Absent in 1 g | |

*Dissolution profile was modified prior to the 12-Month timepoint due to lack of utility of 45 minute reading, and also to capture infinity spin data.

TABLE 54
Stability Data for Compound 1 SDD GMP Drug Product, 5 mg Stored at 40° C./75% RH

| | Acceptance | Storage Time | | | |
|---|---|---|---|---|---|
| Test | Criteria | 0 Month | 1 Month | 3 Months | 6 Months |
| Appearance | White to off-white | Off-white | Off-white | White powder | White powder |
| | to tan powder in | powder in an | powder in an | in an opaque | in an opaque |
| | an opaque white | opaque white | opaque white | white capsule | white capsule |
| | capsule | capsule | capsule | | |
| Assay | 90.0-110.0% | 99.0 | 96.7 | 97.9 | 96.6 |
| | Label Claim | | | | |
| Impurities | Individual ≤0.5% | RRT 1.05: 0.05 | RRT 1.05: 0.05 | RRT 1.05: 0.05 | RRT 1.05: 0.05 |
| | Report RRT and | Total: 0.05 | RRT 1.17: 0.05 | Total: 0.05 | Total: 0.05 |
| | area % of all | | Total: 0.10 | | |
| | impurities ≥0.05% | | | | |
| | Total Impurities ≤3.0% | | | | |
| Water | Report Results | 3.4 | 2.5 | 2.9 | 2.5 |
| Content | (wt %) | | | | |
| Dissolution | Report Results | 15 min: 83 | 15 min: 98 | 15 min: 72 | 15 min: 82 |
| | (%) | 30 min: 94 | 30 min: 101 | 30 min: 89 | 30 min: 96 |
| | | 45 min: 97 | 45 min: 101 | 45 min: 92 | 45 min: 98 |
| | | 60 min: 98 | 60 min: 102 | 60 min:96 | 60 min:99 |
| Microbial | TAMC ≤10³ | <50 CFU/g | Not Scheduled | Not Scheduled | Not Scheduled |
| Limits | CFU/g | <50 CFU/g | | | |
| | TYMC ≤10² | Absent in 1 g | | | |
| | CFU/g | | | | |
| | E. coli Absent/g | | | | |

TABLE 55
Stability Data for Compound 1 SDD GMP Drug
Product, 25 mg Stored at 25 ° C./60% RH

| | Acceptance | Storage Time | | |
|---|---|---|---|---|
| Test | Criteria | 0 Month | 1 Month | 3 Months |
| Appearance | White to | Off-white | Off-white | Off-white |
| | off-white to | powder | powder | powder |
| | tan powder | in an | in an | in an |
| | in an opaque | opaque | opaque | opaque |
| | white | white | white | white |
| | capsule | capsule | capsule | capsule |
| Assay | 90.0-110.0% | 101.3 | 99.9 | 101.2 |
| | Label Claim | | | |
| Impurities | Individual ≤0.5% | RRT 1.05: 0.06 | RRT 1.05: 0.07 | RRT 1.05: 0.06 |
| | Report RRT and | Total: 0.06 | Total: 0.07 | Total: 0.06 |
| | area % of all | | | |
| | impurities ≥0.05% | | | |
| | Total | | | |
| | Impurities ≤3.0% | | | |
| Water | Report | 2.9 | 2.2 | 2.4 |
| Content | Results | | | |
| | (wt %) | | | |
| Dissolution* | Report | 15 min: 83 | 15 min: 93 | 15 min: 94 |
| | Results | 30 min: 97 | 30 min: 102 | 30 min: 99 |
| | (%) | 45 min: 99 | 45 min: 104 | 45 min: 102 |
| | | 60 min: 101 | 60 min: 105 | 60 min: 103 |
| Microbial | TAMC ≤10³ CFU/g | <50 CFU/g | Not | Not |
| Limits | TYMC ≤10² CFU/g | <50 CFU/g | Scheduled | Scheduled |
| | E. coli Absent/g | Absent in 1 g | | |

| | Storage Time | | | |
|---|---|---|---|---|
| Test | 6 Months | 9 Months | 12 Months* | 18 Months |
| Appearance | White | White | White | White |
| | powder | powder | powder | powder |
| | in an | in an | in an | in an |
| | opaque | opaque | opaque | opaque |
| | white | white | white | white |
| | capsule | capsule | capsule | capsule |
| Assay | 101.9 | 100.8 | 101.2 | 100.5 |
| Impurities | RRT1.04: 0.06 | RRT 1.05: 0.05 | RRT 1.05: 0.05 | RRT 1.05: 0.05 |
| | Total: 0.06 | Total: 0.05 | Total: 0.05 | Total: 0.05 |
| Water | 2.7 | 2.1 | 2.2 | 2.6 |
| Content | | | | |
| Dissolution* | 15 min: 94 | 15 min: 85 | 15 min: 88 | 15 min: 85 |
| | 30 min: 102 | 30 min: 94 | 30 min: 97 | 30 min: 101 |
| | 45 min: 104 | 45 min: 96 | 60 min: 101 | 60 min: 100 |
| | 60 min: 104 | 60 min: 100 | 75 min: 102 | 75 min: 102 |
| Microbial | Not | Not | <100 CFU/g | Not |
| Limits | Scheduled | Scheduled | <50 CFU/g | Scheduled |
| | | | Absent in 1 g | |

*Dissolution profile was modified prior to the 12-Month timepoint due to lack of utility of 45 minute reading, and also to capture infinity spin data.

TABLE 56
Stability Data for Compound 1 SDD GMP Drug Product, 25 mg at 40° C./75% RH

| | Acceptance | Storage Time | | | |
|---|---|---|---|---|---|
| Test | Criteria | 0 Month | 1 Month | 3 Months | 6 Months |
| Appearance | White to off-white | Off-white | Off-white | Off-white | White powder |
| | to tan powder in | powder in an | powder in an | powder in an | in an opaque |
| | an opaque white | opaque white | opaque white | opaque white | white capsule |
| | capsule | capsule | capsule | capsule | |
| Assay | 90.0-110.0% | 101.3 | 101.7 | 100.7 | 101.2 |
| | Label Claim | | | | |
| Impurities | Individual ≤0.5% | RRT 1.05: 0.06 | RRT 1.05: 0.05 | RRT 1.05: 0.05 | RRT 1.04: 0.05 |
| | Report RRT and | Total: 0.06 | Total: 0.05 | Total: 0.05 | RRT 1.53: 0.05 |
| | area % of all | | | | Total: 0.10 |
| | impurities ≥0.05% | | | | |
| | Total Impurities ≤3.0% | | | | |
| Water | Report Results | 2.9 | 2.2 | 2.7 | 3.1 |
| Content | (wt %) | | | | |
| Dissolution | Report Results | 15 min: 83 | 15 min: 85 | 15 min: 91 | 15 min: 92 |
| | (%) | 30 min: 97 | 30 min: 96 | 30 min: 101 | 30 min: 101 |
| | | 45 min: 99 | 45 min: 101 | 45 min: 103 | 45 min: 103 |
| | | 60 min: 101 | 60 min: 102 | 60 min: 105 | 60 min: 104 |
| Microbial | TAMC ≤10³ | <50 CFU/g | Not Scheduled | Not Scheduled | Not Scheduled |
| Limits | CFU/g | <50 CFU/g | | | |
| | TYMC ≤10² | Absent in 1 g | | | |
| | CFU/g | | | | |
| | E. coli Absent/g | | | | |

TABLE 57
Stability Data for Compound 1 SDD GMP Drug
Product, 50 mg Stored at 25° C./60% RH

| | Acceptance | Storage Time | | |
|---|---|---|---|---|
| Test | Criteria | 0 Month | 1 Month | 3 Months |
| Appearance | White to | Off-white | Off-white | Off-white |
| | off-white to | powder | powder | powder |
| | tan powder | in an | in an | in an |
| | in an opaque | opaque | opaque | opaque |
| | white | white | white | white |
| | capsule | capsule | capsule | capsule |
| Assay | 90.0-110.0% | 99.6 | 100.6 | 98.8 |
| | Label Claim | | | |
| Impurities | Individual ≤0.5% | RRT 1.05: 0.05 | RRT 1.05: 0.06 | RRT 1.05: 0.07 |
| | Report RRT and | Total: 0.05 | Total: 0.06 | Total: 0.07 |
| | area % of all | | | |
| | impurities ≥0.05% | | | |
| | Total | | | |
| | Impurities ≤3.0% | | | |
| Water | Report | 2.6 | 2.1 | 2.5 |
| Content | Results | | | |
| | (wt %) | | | |
| Dissolution* | Report | 15 min: 72 | 15 min: 72 | 15 min: 72 |
| | Results | 30 min: 93 | 30 min: 96 | 30 min: 94 |
| | (%) | 45 min: 98 | 45 min: 103 | 45 min: 100 |
| | | 60 min: 101 | 60 min: 104 | 60 min: 103 |
| Microbial | TAMC ≤10³ CFU/g | <50 CFU/g | Not | Not |
| Limits | TYMC ≤10² CFU/g | <50 CFU/g | Scheduled | Scheduled |
| | E. coli Absent/g | Absent in 1 g | | |

| | Storage Time | | | |
|---|---|---|---|---|
| Test | 6 Months | 9 Months | 12 Months* | 18 Months |
| Appearance | White | White | White | White |
| | powder | powder | powder | powder |
| | in an | in an | in an | in an |
| | opaque | opaque | opaque | opaque |
| | white | white | white | white |
| | capsule | capsule | capsule | capsule |
| Assay | 100.6 | 103.0 | 101.8 | 100.6 |
| Impurities | RRT 1.04: 0.06 | RRT 1.05: 0.05 | RRT 1.05: 0.05 | RRT 1.05: 0.05 |
| | RRT 1.53: 0.06 | Total: 0.05 | Total: 0.05 | Total: 0.05 |
| | Total: 0.11 | | | |
| Water | 2.1 | 2.0 | 2.2 | 2.5 |
| Content | | | | |
| Dissolution* | 15 min: 83 | 15 min: 80 | 15 min: 76 | 15 min: 84 |
| | 30 min: 99 | 30 min: 96 | 30 min: 95 | 30 min: 99 |
| | 45 min: 102 | 45 min: 100 | 60 min: 99 | 60 min: 102 |
| | 60 min: 102 | 60 min: 100 | 75 min: 100 | 75 min: 103 |
| Microbial | Not | Not | <100 CFU/g | Not |
| Limits | scheduled | scheduled | <50 CFU/g | scheduled |
| | | | Absent in 1 g | |

*Dissolution profile was modified prior to the 12-Month timepoint due to lack of utility of 45 minute reading, and also to capture infinity spin data.

TABLE 58
Stability Data for Compound 1 SDD GMP Drug Product, 50 mg Stored at 40° C./75% RH

| | Acceptance | Storage Time | | | |
|---|---|---|---|---|---|
| Test | Criteria | 0 Month | 1 Month | 3 Months | 6 Months |
| Appearance | White to off-white | Off-white | Off-white | Off-white | Off-white |
| | to tan powder in | powder in an | powder in an | powder in an | powder in an |
| | an opaque white | opaque white | opaque white | opaque white | opaque white |
| | capsule | capsule | capsule | capsule | capsule |
| Assay | 90.0-110.0% | 99.6 | 102.6 | 101.4 | 103.5 |
| | Label Claim | | | | |
| Impurities | Individual ≤0.5% | RRT 1.05: 0.05 | RRT 1.05: 0.05 | RRT 1.05: 0.05 | RRT 1.04: 0.06 |
| | Report RRT and | Total: 0.05 | Total: 0.05 | Total: 0.05 | Total: 0.06 |
| | area % of all | | | | |
| | impurities ≥0.05% | | | | |
| | Total Impurities ≤3.0% | | | | |
| Water | Report Results | 2.6 | 2.1 | 3.5 | 3.5 |
| Content | (wt %) | | | | |
| Dissolution | Report Results | 15 min: 72 | 15 min: 77 | 15 min: 62 | 15 min: 62 |
| | (%) | 30 min: 93 | 30 min: 9 | 30 min: 90 | 30 min: 90 |
| | | 45 min: 98 | 45 min: 103 | 45 min: 99 | 45 min: 99 |
| | | 60 min: 101 | 60 min: 104 | 60 min: 103 | 60 min: 103 |
| Microbial | TAMC ≤10³ | <50 CFU/g | Not Scheduled | Not Scheduled | Not Scheduled |
| Limits | CFU/g | <50 CFU/g | | | |
| | TYMC ≤10² | Absent in 1 g | | | |
| | CFU/g | | | | |
| | E. coli Absent/g | | | | |

TABLE 59
Stability Data for Compound 1 SDD GMP Drug Product, 2 mg Lot at 25° C/60% RH

| | Acceptance | Storage Time | | | |
|---|---|---|---|---|---|
| Test | Criteria | 0 Month | 1 Month | 3 Months | 6 Months |
| Appearance | White to off-white | White powder | White powder | White powder | White powder |
| | to tan powder in | in an opaque | in an opaque | in an opaque | in an opaque |
| | an opaque white | white capsule | white capsule | white capsule | white capsule |
| | capsule | | | | |
| Assay | 90.0-110.0% | 100.4 | 101.9 | 98.7 | 102.1 |
| | Label Claim | | | | |
| Impurities | Individual ≤0.5% | RRT: 0.85a: <0.05 | RRT 0.85: 0.05 | RRT 0.40: 0.14 | RRT 0.85: <0.05 |
| | Report RRT and | RRT 0.85b: <0.05 | RRT 1.05: <0.05 | RRT 0.85: <0.05 | RRT 1.05: <0.05 |
| | area % of all | RRT 1.05: <0.05 | Total: 0.05 | RRT 1.05: 0.08 | RRT 1.15: <0.05 |
| | impurities ≥0.05% | Total: <0.05 | | RRT 1.34: <0.05 | Total: <0.05 |
| | Total Impurities ≤3.0% | | | Total: 0.23 | |
| Water | Report Results | 3.3 | 3.2 | 3.1 | 3.5 |
| Content | (wt %) | | | | |
| Dissolution | Report Results | 15 min: 95 | 15 min: 94 | 15 min: 93 | 15 min: 97 |
| | (%) | 30 min: 95 | 30 min: 92 | 30 min: 93 | 30 min: 97 |
| | | 60 min: 95 | 60 min: 95 | 60 min: 94 | 60 min: 97 |
| | | 75 min: 96 | 75 min: 96 | 75 min: 94 | 75 min: 97 |
| Microbial | TAMC ≤10³ | <10 CFU/g | Not Scheduled | Not Scheduled | Not Scheduled |
| Limits | CFU/g | <10 CFU/g | | | |
| | TYMC ≤10² | Absent in 1 g | | | |
| | CFU/g | | | | |
| | E. coli Absent/g | | | | |

TABLE 60
Stability Data for Compound 1 SDD GMP Drug Product, 2 mg Stored at 25° C./60% RH

| | Acceptance | Storage Time | | | |
|---|---|---|---|---|---|
| Test | Criteria | 0 Month | 1 Month | 3 Months | 6 Months |
| Appearance | White to off-white | White powder | White powder | White powder | White powder |
| | to tan powder in | in an opaque | in an opaque | in an opaque | in an opaque |
| | an opaque white | white capsule | white capsule | white capsule | white capsule |
| | capsule | | | | |
| Assay | 90.0-110.0% | 100.4 | 101.9 | 98.7 | 102.1 |
| | Label Claim | | | | |
| Impurities | Individual ≤0.5% | RRT: 0.85a: <0.05 | RRT 0.85: 0.05 | RRT 0.40: 0.14 | RRT 0.85: <0.05 |
| | Report RRT and | RRT 0.85b: <0.05 | RRT 1.05: <0.05 | RRT 0.85: <0.05 | RRT 1.05: <0.05 |
| | area % of all | RRT 1.05: <0.05 | Total: 0.05 | RRT 1.05: 0.08 | RRT 1.15: <0.05 |
| | impurities ≥0.05% | Total: <0.05 | | RRT 1.34: <0.05 | Total: <0.05 |
| | Total Impurities ≤3.0% | | | Total: 0.23 | |
| Water | Report Results | 3.3 | 3.2 | 3.1 | 3.5 |
| Content | (wt %) | | | | |
| Dissolution | Report Results | 15 min: 95 | 15 min: 94 | 15 min: 93 | 15 min: 97 |
| | (%) | 30 min: 95 | 30 min: 92 | 30 min: 93 | 30 min: 97 |
| | | 60 min: 95 | 60 min: 95 | 60 min: 94 | 60 min: 97 |
| | | 75 min: 96 | 75 min: 96 | 75 min: 94 | 75 min: 97 |
| Microbial | TAMC ≤10³ | <10 CFU/g | Not Scheduled | Not Scheduled | Not Scheduled |
| Limits | CFU/g | <10 CFU/g | | | |
| | TYMC ≤10² | Absent in 1 g | | | |
| | CFU/g | | | | |
| | E. coli Absent/g | | | | |

TABLE 61
Stability Data for Compound 1 SDD GMP Drug Product, 2 mg Stored at 40° C./75% RH

| | Acceptance | Storage Time | | | |
|---|---|---|---|---|---|
| Test | Criteria | 0 Month | 1 Month | 3 Months | 6 Months |
| Appearance | White to off-white | White powder | White powder | White powder | White powder |
| | to tan powder in | in an opaque | in an opaque | in an opaque | in an opaque |
| | an opaque white | white capsule | white capsule | white capsule | white capsule |
| | capsule | | | | |
| Assay | 90.0-110.0% | 100.4 | 102.1 | 95.2 | 98.7 |
| | Label Claim | | | | |
| Impurities | Individual ≤0.5% | RRT 0.85a: <0.05 | RRT 0.85: 0.05 | RRT 0.84: <0.05 | RRT 0.85: <0.05 |
| | Report RRT and | RRT 0.85b: <0.05 | RRT 1.05: <0.05 | RRT 1.05: 0.10 | RRT 0.89: <0.05 |
| | area % of all | RRT 1.05: <0.05 | Total: 0.05 | RRT 1.15: <0.05 | RRT 1.05: <0.05 |
| | impurities ≥0.05% | Total: <0.05 | | Total: 0.10 | RRT 1.15: 0.05 |
| | Total Impurities ≤3.0% | | | | Total 0.05 |
| Water | Report Results | 3.3 | 3.4 | 3.7 | 4.1 |
| Content | (wt %) | | | | |
| Dissolution | Report Results | 15 min: 95 | 15 min: 92 | 15 min: 95 | 15 min: 94 |
| | (%) | 30 min: 95 | 30 min: 89 | 30 min: 95 | 30 min: 95 |
| | | 60 min: 95 | 60 min: 92 | 60 min: 96 | 60 min: 95 |
| | | 75 min: 96 | 75 min: 94 | 75 min: 96 | 75 min: 95 |
| Microbial | TAMC ≤10³ | <10 CFU/g | Not Scheduled | Not Scheduled | Not Scheduled |
| Limits | CFU/g | <10 CFU/g | | | |
| | TYMC ≤10² | Absent in 1 g | | | |
| | CFU/g | | | | |
| | E. coli Absent/g | | | | |

TABLE 62
Stability Data for Compound 1 SDD GMP Drug Product, 5 mg Stored at 25° C./60% RH

| | Acceptance | Storage Time | | | |
|---|---|---|---|---|---|
| Test | Criteria | 0 Month | 1 Month | 3 Months | 6 Months |
| Appearance | White to off-white | White powder | White powder | White powder | White powder |
| | to tan powder in | in an opaque | in an opaque | in an opaque | in an opaque |
| | an opaque white | white capsule | white capsule | white capsule | white capsule |
| | capsule | | | | |
| Assay | 90.0-110.0% | 98.8 | 100.5 | 93.3 | 101.1 |
| | Label Claim | | | | |
| Impurities | Individual ≤0.5% | RRT: 0.84: <0.05 | RRT 0.85: 0.05 | RRT 0.84: <0.05 | RRT 0.85: <0.05 |
| | Report RRT and | RRT 0.85: <0.05 | RRT 1.05: <0.05 | RRT 1.05: 0.10 | RRT 1.05: <0.05 |
| | area % of all | RRT 1.05: <0.05 | Total: 0.05 | Total: 0.10 | Total: <0.05 |
| | impurities ≥0.05% | Total: <0.05 | | | |
| | Total Impurities ≤3.0% | | | | |
| Water | Report Results | 3.3 | 3.2 | 3.1 | 3.6 |
| Content | (wt %) | | | | |
| Dissolution | Report Results | 15 min: 96 | 15 min: 97 | 15 min: 95 | 15 min: 95 |
| | (%) | 30 min: 95 | 30 min: 97 | 30 min: 95 | 30 min: 95 |
| | | 60 min: 96 | 60 min: 97 | 60 min: 95 | 60 min: 95 |
| | | 75 min: 96 | 75 min: 97 | 75 min: 96 | 75 min: 95 |
| Microbial | TAMC <10³ | <50 CFU/g | Not Scheduled | Not Scheduled | Not Scheduled |
| Limits | CFU/g | <50 CFU/g | | | |
| | TYMC ≤10² | Absent in 1 g | | | |
| | CFU/g | | | | |
| | E. coli Absent/g | | | | |

TABLE 63
Stability Data for Compound 1 SDD GMP Drug Product, 15 mg Stored at 25° C./60% RH

| | Acceptance | Storage Time | | | |
|---|---|---|---|---|---|
| Test | Criteria | 0 Month | 1 Month | 3 Months | 6 Months |
| Appearance | White to off-white | White powder | White powder | White powder | White powder |
| | to tan powder in | in an opaque | in an opaque | in an opaque | in an opaque |
| | an opaque white | white capsule | white capsule | white capsule | white capsule |
| | capsule | | | | |
| Assay | 90.0-110.0% | 101.0 | 103.4 | 95.8 | 98.3 |
| | Label Claim | | | | |
| Impurities | Individual ≤0.5% | RRT: 0.85a: <0.05 | RRT 0.85: 0.05 | RRT 0.84: <0.05 | RRT 0.85: <0.05 |
| | Report RRT and | RRT 0.85b: <0.05 | RRT 1.05: <0.05 | RRT 1.05: 0.08 | RRT 1.05: <0.05 |
| | area % of all | RRT 1.05: <0.05 | Total: 0.05 | Total: 0.08 | Total: <0.05 |
| | impurities ≥0.05% | Total: <0.05 | | | |
| | Total Impurities ≤3.0% | | | | |
| Water | Report Results | 2.9 | 2.7 | 2.7 | 3.2 |
| Content | (wt %) | | | | |
| Dissolution | Report Results | 15 min: 94 | 15 min: 92 | 15 min: 95 | 15 min: 96 |
| | (%) | 30 min: 99 | 30 min: 94 | 30 min: 96 | 30 min: 98 |
| | | 60 min: 100 | 60 min: 96 | 60 min: 97 | 60 min: 98 |
| | | 75 min: 100 | 75 min: 97 | 75 min: 97 | 75 min: 98 |
| Microbial | TAMC ≤10³ | <10 CFU/g | Not Scheduled | Not Scheduled | Not Scheduled |
| Limits | CFU/g | <10 CFU/g | | | |
| | TYMC ≤10² | Absent in 1 g | | | |
| | CFU/g | | | | |
| | E. coli Absent/g | | | | |

TABLE 64
Stability Data for Compound 1 SDD GMP Drug Product, 15 mg Stored at 40° C./75% RH

| | Acceptance | Storage Time | | | |
|---|---|---|---|---|---|
| Test | Criteria | 0 Month | 1 Month | 3 Months | 6 Months |
| Appearance | White to off-white | White powder | White powder | White powder | White powder |
| | to tan powder in | in an opaque | in an opaque | in an opaque | in an opaque |
| | an opaque white | white capsule | white capsule | white capsule | white capsule |
| | capsule | | | | |
| Assay | 90.0-110.0% | 101.0 | 100.4 | 96.3 | 96.3 |
| | Label Claim | | | | |
| Impurities | Individual ≤0.5% | RRT: 0.85a: <0.05 | RRT 0.85: 0.06 | RRT 0.84: <0.05 | RRT 0.85: <0.05 |
| | Report RRT and | RRT 0.85b: <0.05 | RRT 1.05: <0.05 | RRT 1.05: 0.26 | RRT 1.05: <0.05 |
| | area % of all | RRT 1.05: <0.05 | Total: 0.06 | RRT 1.61: 0.06 | Total: <0.05 |
| | impurities ≥0.05% | Total: <0.05 | | Total: 0.32 | |
| | Total Impurities ≤3.0% | | | | |
| Water | Report Results | 2.9 | 2.9 | 3.3 | 3.9 |
| Content | (wt %) | | | | |
| Dissolution | Report Results | 15 min: 94 | 15 min: 91 | 15 min: 92 | 15 min: 87 |
| | (%) | 30 min: 99 | 30 min: 93 | 30 min: 93 | 30 min: 94 |
| | | 60 min: 100 | 60 min: 95 | 60 min: 94 | 60 min: 96 |
| | | 75 min: 100 | 75 min: 95 | 75 min: 94 | 75 min: 96 |
| Microbial | TAMC ≤10³ | <10 CFU/g | Not Scheduled | Not Scheduled | Not Scheduled |
| Limits | CFU/g | <10 CFU/g | | | |
| | TYMC ≤10² | Absent in 1 g | | | |
| | CFU/g | | | | |
| | E. coli Absent/g | | | | |

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

We claim:

1. A pharmaceutical composition, formulation, or unit dosage form comprising:
(a) Compound 1: 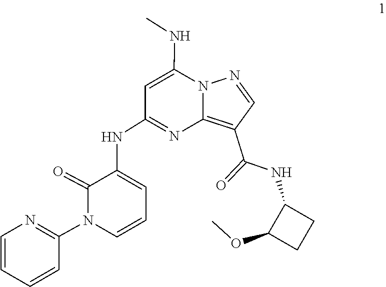 [SMILES: CNc1cc(Nc2cccn(-c3ccccn3)c2=O)nc2c(C(=O)N[C@@H]3CC[C@H]3OC)cnn12]
or a solvate thereof, or a pharmaceutically acceptable salt thereof;
(b) one or more fillers or diluents;
(c) one or more solubilizers; and
(d) one or more binders.

2. The pharmaceutical composition, formulation, or unit dosage form of claim 1, wherein (a) comprises Compound 1.

3. The pharmaceutical composition, formulation, or unit dosage form of claim 1 or 2, wherein the solubilizer comprises one or more solubilizers of formula I-A: 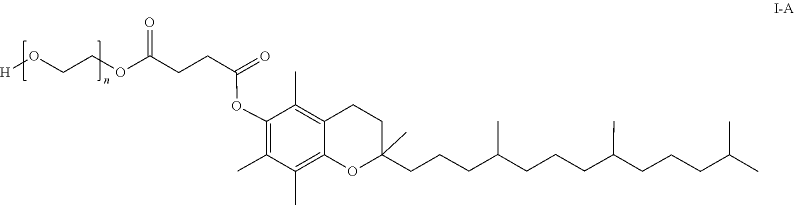
or a pharmaceutically acceptable salt thereof, wherein n is from about 10 to about 50, about 50 to about 100, about 100 to about 200, about 200 to about 500, about 500 to about 1,000, about 1,000 to about 2,000, about 2,000 to about 5,000, or about 5,000 to about 10,000.

4. The pharmaceutical composition, formulation, or unit dosage form of any one of claims 1-3, wherein the solubilizer comprises Vitamin E TPGS.

5. The pharmaceutical composition, formulation, or unit dosage form of any one of claims 1-4, wherein the filler or diluent comprises microcrystalline cellulose.

6. The pharmaceutical composition, formulation, or unit dosage form of any one of claims 1-5, wherein the filler or diluent comprises Avicel PH101.

7. The pharmaceutical composition, formulation, or unit dosage form of any one of claims 1-6, wherein the binder comprises povidone.

8. The pharmaceutical composition, formulation, or unit dosage form of any one of claims 1-7, wherein the binder comprises PVP K30.

9. The pharmaceutical composition, formulation, or unit dosage form of any one of claims 1-8, comprising an amount of Compound 1 between about 1 mg to about 2.5 mg, about 2.5 mg to about 5 mg, about 5 mg to about 7.5 mg, about 7.5 mg to about 10 mg, about 10 mg to about 12.5 mg, about 12.5 mg to about 15 mg, about 15 mg to about 17.5 mg, about 17.5 mg to about 20 mg, about 20 mg to about 22.5 mg, about 22.5 mg to about 25 mg, about 25 mg to about 27.5 mg, about 27.5 mg to about 30 mg, about 30 mg to about 32.5 mg, about 32.5 mg to about 35 mg, about 35 mg to about 37.5 mg, about 37.5 mg to about 40 mg, about 40 mg to about 45 mg, or about 45 mg to about 50 mg.

10. The pharmaceutical composition, formulation, or unit dosage form of any one of claims 1-9, comprising about 2 mg of Compound 1.

11. The pharmaceutical composition, formulation, or unit dosage form of any one of claims 1-9, comprising about 5 mg of Compound 1.

12. The pharmaceutical composition, formulation, or unit dosage form of any one of claims 1-9, comprising about 15 mg of Compound 1.

13. The pharmaceutical composition, formulation, or unit dosage form of any one of claims 1-9, comprising about 25 mg of Compound 1.

14. The pharmaceutical composition, formulation, or unit dosage form of any one of claims 1-9, comprising about 50 mg of Compound 1.

15. The pharmaceutical composition, formulation, or unit dosage form of any one of claims 1-14, comprising an amount of diluent between about 15 mg to about 17.5 mg, about 17.5 mg to about 20 mg, about 20 mg to about 22.5 mg, about 22.5 mg to about 25 mg, about 25 mg to about 27.5 mg, about 27.5 mg to about 30 mg, about 30 mg to about 32.5 mg, about 32.5 mg to about 35 mg, about 35 mg to about 37.5 mg, about 37.5 mg to about 40 mg, about 40 mg to about 45 mg, about 45 mg to about 50 mg, about 50 mg to about 75 mg, about 75 mg to about 100 mg, about 100 mg to about 125 mg, about 125 mg to about 150 mg, about 150 mg to about 175 mg, about 175 mg to about 200 mg, or about 200 mg to about 225 mg.

16. The pharmaceutical composition, formulation, or unit dosage form of any one of claims 1-15, comprising about 34 mg of filler or diluent.

17. The pharmaceutical composition, formulation, or unit dosage form of any one of claims 1-15, comprising about 170 mg of filler or diluent.

18. The pharmaceutical composition, formulation, or unit dosage form of any one of claims 15-17, wherein the filler or diluent comprises microcrystalline cellulose.

19. The pharmaceutical composition, formulation, or unit dosage form of any one of claims 1-18, comprising an amount of solubilizer between about 1 mg to about 2.5 mg, about 2.5 mg to about 5 mg, about 5 mg to about 7.5 mg, about 7.5 mg to about 10 mg, about 10 mg to about 12.5 mg, about 12.5 mg to about 15 mg, about 15 mg to about 17.5 mg, about 17.5 mg to about 20 mg, about 20 mg to about 22.5 mg, about 22.5 mg to about 25 mg, about 25 mg to about 27.5 mg, about 27.5 mg to about 30 mg, about 30 mg to about 32.5 mg, about 32.5 mg to about 35 mg, about 35 mg to about 37.5 mg, about 37.5 mg to about 40 mg, about 40 mg to about 45 mg, about 45 mg to about 50 mg, about 50 mg to about 75 mg, about 75 mg to about 100 mg, about 100 mg to about 125 mg, or about 125 mg to about 150 mg.

20. The pharmaceutical composition, formulation, or unit dosage form of any one of claims 1-19, comprising 10 mg of solubilizer.

21. The pharmaceutical composition, formulation, or unit dosage form of any one of claims 1-19, comprising 50 mg of solubilizer.

22. The pharmaceutical composition, formulation, or unit dosage form of any one of claims 19-21, wherein the solubilizer comprises Vitamin E TPGS.

23. The pharmaceutical composition, formulation, or unit dosage form of any one of claims 1-22, comprising an amount of binder between about 0.25 mg to about 0.5 mg, about 0.5 mg to about 0.75 mg, about 0.75 mg to about 1 mg, about 1 mg to about 2 mg, about 2 mg to about 3 mg, about 3 mg to about 4 mg, about 4 mg to about 5 mg, about 5 mg to about 6 mg, about 6 mg to about 7 mg, about 7 mg to about 8 mg, about 8 mg to about 9 mg, or about 9 mg to about 10 mg.

24. The pharmaceutical composition, formulation, or unit dosage form of any one of claims 1-23, comprising 1 mg of binder.

25. The pharmaceutical composition, formulation, or unit dosage form of any one of claims 1-23, comprising 5 mg of binder.

26. The pharmaceutical composition, formulation, or unit dosage form of any one of claims 23-25, wherein the binder comprises povidone.

27. The pharmaceutical composition, formulation, or unit dosage form of any one of claims 1-8, comprising:
(a) between about 2.5 mg to about 7.5 mg of Compound 1;
(b) between about 17 mg to about 51 mg of filler or diluent;
(c) between about 5 mg to about 15 mg of solubilizer; and
(d) between about 0.5 mg to about 1.5 mg binder.

28. The pharmaceutical composition, formulation, or unit dosage form of any one of claims 1-8, comprising (a), (b), (c), and (d) of:
(a) between about 2.5 mg to about 7.5 mg of Compound 1;
(b) between about 17 mg to about 51 mg of microcrystalline cellulose;
(c) between about 5 mg to about 15 mg of Vitamin E TPGPS; and
(d) between about 0.5 mg to about 1.5 mg povidone.

29. The pharmaceutical composition, formulation, or unit dosage form of any one of claims 1-8, comprising (a), (b), (c), and (d) of:
(a) about 5 mg of Compound 1;
(b) about 34 mg of microcrystalline cellulose;
(c) about 10 mg of Vitamin E TPGS; and
(d) about 1 mg povidone.

30. The pharmaceutical composition, formulation, or unit dosage form of any one of claims 1-8, comprising (a), (b), (c), and (d) of:
(a) between about 12.5 mg to about 37.5 mg of Compound 1;
(b) between about 85 mg to about 255 mg of a filler or diluent;
(c) between about 25 mg to about 75 mg of a solubilizer; and
(d) between about 2.5 mg to about 7.5 mg a binder.

31. The pharmaceutical composition, formulation, or unit dosage form of any one of claims 1-8, comprising (a), (b), (c), and (d) of:
(a) about 25 mg of Compound 1;
(b) about 170 mg of a filler or diluent;
(c) about 10 mg of solubilizer; and
(d) about 5 mg of binder.

32. The pharmaceutical composition, formulation, or unit dosage form of any one of claims 1-8, comprising (a), (b), (c), and (d) of:
(a) between about 12.5 mg to about 37.5 mg of Compound 1;
(b) between about 85 mg to about 255 mg of microcrystalline cellulose;
(c) between about 25 mg to about 75 mg of Vitamin E TPGS; and
(d) between about 2.5 mg to about 7.5 mg povidone.

33. The pharmaceutical composition, formulation, or unit dosage form of any one of claims 1-8, comprising (a), (b), (c), and (d) of:
(a) about 25 mg of Compound 1;
(b) about 170 mg of microcrystalline cellulose;
(c) about 10 mg of Vitamin E TPGS; and
(d) about 5 mg of povidone.

34. The pharmaceutical composition, formulation, or unit dosage form of any one of claims 1-8, comprising:
a) between about 5% to about 15% (w/w) of Compound 1;
b) between about 32% to about 80% (w/w) of a filler or diluent;
c) between about 10% to about 30% (w/w) of a solubilizer; and
d) between about 1% to about 3% (w/w) of a binder.

35. The pharmaceutical composition, formulation, or unit dosage form of any one of claims 1-8, comprising:
a) about 10% (w/w) of Compound 1;
b) about 68% (w/w) of a filler or diluent;
c) about 20% (w/w) of a solubilizer; and
d) about 2% (w/w) of a binder.

36. The pharmaceutical composition, formulation, or unit dosage form of any one of claims 1-8, comprising:
a) about 20% (w/w) of Compound 1;
b) about 58% (w/w) of a filler or diluent;
c) about 20% (w/w) of a solubilizer; and
d) about 2% (w/w) of a binder.

37. The pharmaceutical composition, formulation, or unit dosage form of any one of claims 1-8, comprising:
a) about 30% (w/w) of Compound 1;
b) about 48% (w/w) of a filler or diluent;
c) about 20% (w/w) of a solubilizer; and
d) about 2% (w/w) of a binder.

38. The pharmaceutical composition, formulation, or unit dosage form of any one of claims 1-8, comprising:
a) about 40% (w/w) of Compound 1;
b) about 38% (w/w) of a filler or diluent;
c) about 20% (w/w) of a solubilizer; and
d) about 2% (w/w) of a binder.

39. The pharmaceutical composition, formulation, or unit dosage form of any one of claims 27-38, wherein the diluent is microcrystalline cellulose, the solubilizer is Vitamin E TPGS, and the binder is povidone.

40. A pharmaceutical composition, formulation, or unit dosage form comprising Compound 1: 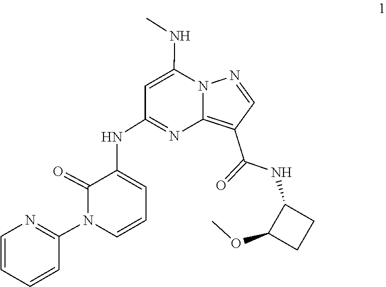 [SMILES: CNc1cc(Nc2cccn(-c3ccccn3)c2=O)nc2c(C(=O)N[C@@H]3CC[C@H]3OC)cnn12]
or a solvate thereof, or a pharmaceutically acceptable salt thereof;
wherein Compound 1 is in the form of a spray-dried dispersion (SDD).

41. The pharmaceutical composition, formulation, or unit dosage form of claim 40, wherein the SDD comprises a cellulosic polymer selected from hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate, carboxymethyl ethyl cellulose, hydroxypropyl methyl cellulose phthalate, cellulose acetate phthalate, cellulose acetate trimellitate, and carboxymethyl ethyl cellulose.

42. The pharmaceutical composition, formulation, or unit dosage form of claim 40, wherein the SDD comprises an about 2:1 to about 1:2 (w/w) mixture of Compound 1 with HPMC-AS-M (hydroxypropylmethylcellulose-acetate, succinate, Grade M).

43. The pharmaceutical composition, formulation, or unit dosage form of any one of claims 40-42, comprising:
a) about 1-4 mg of Compound 1 or a solvate or a pharmaceutically acceptable salt thereof, as an about 2:1 to about 1:2 (w/w) mixture of Compound 1 with HPMC-AS;
b) about 35-140 mg of a first filler comprising mannitol;
c) optionally, about 35-140 mg of a second filler comprising microcrystalline cellulose;
d) about 4-18 mg of a disintegrant comprising croscarmellose sodium;
e) about 0.5-3.0 mg of a glidant comprising colloidal silicon dioxide;
f) about 0.4-2.0 mg of a lubricant comprising sodium stearyl fumarate; and
g) optionally, about 0.5-2 mg of a wetting agent comprising sodium lauryl sulfate.

44. The pharmaceutical composition, formulation, or unit dosage form of any one of claims 40-42, comprising:
a) about 2.5-10 mg of Compound 1 or a solvate or a pharmaceutically acceptable salt thereof, as an about 2:1 to about 1:2 (w/w) mixture of Compound 1 with HPMC-AS;
b) about 30-130 mg of a first filler comprising mannitol;
c) optionally, about 30-130 mg of a second filler comprising microcrystalline cellulose;
d) about 4-20 mg of a disintegrant comprising croscarmellose sodium;
e) about 0.5-4.0 mg of a glidant comprising colloidal silicon dioxide;
f) optionally, about 0.5-4.0 mg of a wetting agent comprising sodium lauryl sulfate; and
g) about 0.4-2.5 mg of a lubricant comprising sodium stearyl fumarate.

45. The pharmaceutical composition, formulation, or unit dosage form of any one of claims 40-42, comprising:
a) about 7.5-30 mg of Compound 1 or a solvate or a pharmaceutically acceptable salt thereof, as an about 2:1 to about 1:2 (w/w) mixture of Compound 1 with HPMC-AS;
b) about 25-130 mg of a first filler comprising mannitol;
c) optionally, about 25-130 mg of a second filler comprising microcrystalline cellulose;
d) about 4-20 mg of a disintegrant comprising croscarmellose sodium;
e) about 0.5-4.0 mg of a glidant comprising colloidal silicon dioxide;
f) optionally, about 0.5-4.0 mg of a wetting agent comprising sodium lauryl sulfate; and
g) about 0.4-4.0 mg of a lubricant comprising sodium stearyl fumarate.

46. The pharmaceutical composition, formulation, or unit dosage form of any one of claims 40-42, comprising:
a) about 12.5-50 mg of Compound 1 or a solvate or a pharmaceutically acceptable salt thereof, as an about 2:1 to about 1:2 (w/w) mixture of Compound 1 with HPMC-AS;
b) about 20-95 mg of a first filler comprising mannitol;
c) optionally, about 20-95 mg of a second filler comprising microcrystalline cellulose;
d) about 4-20 mg of a disintegrant comprising croscarmellose sodium;
e) about 0.5-4.0 mg of a glidant comprising colloidal silicon dioxide;
f) optionally, about 0.5-4.0 mg of a wetting agent comprising sodium lauryl sulfate; and
g) about 0.4-4.0 mg of a lubricant comprising sodium stearyl fumarate.

47. The pharmaceutical composition, formulation, or unit dosage form of any one of claims 40-42, comprising:
a) about 25-75 mg of Compound 1 or a solvate or a pharmaceutically acceptable salt thereof, as an about 2:1 to about 1:2 (w/w) mixture of Compound 1 with HPMC-AS;
b) about 24-96 mg of a first filler comprising mannitol;
c) optionally, about 24-96 mg of a second filler comprising microcrystalline cellulose;
d) about 6-25 mg of a disintegrant comprising croscarmellose sodium;
e) about 1.0-5.0 mg of a glidant comprising colloidal silicon dioxide;
f) optionally, about 1.0-5.0 mg of a wetting agent comprising sodium lauryl sulfate; and
g) about 0.7-2.6 mg of a lubricant comprising sodium stearyl fumarate.

48. The pharmaceutical composition, formulation, or unit dosage form of any one of claims 43-47, wherein each of b), c), d), and g) are distributed between granules comprising a) and an extragranular portion of the pharmaceutical composition, formulation, or unit dosage form.

49. The pharmaceutical composition, formulation, or unit dosage form of any one of claims 40-47, comprising one of the following % w/w of ingredients shown below:

| | 2 mg | 5 mg | | 15 mg |
|---|---|---|---|---|
| Ingredients | w/w % | w/w % | w/w % | w/w % |
| Spray Dried | 1-5 | 2.5-10.0 | 2.5-10.0 | 7.0-40.0 |
| intermediate | | | | |
| Microcrystalline | 0-80 or | 0-75.0 or | 0-75.0 or | 0-70.0 or |
| Cellulose | 18.0-80.0 | 18.0-75.0 | 18.0-75.0 | 18.0-70.0 |
| Mannitol | 18.0-80.0 | 18.0-75.0 | 18.0-75.0 | 18.0-70.0 |
| Croscarmellose | 4.5-26.0 | 5.0-18.0 | 6.5-27.0 | 4.6-20.0 |
| Sodium | | | | |
| Colloidal Silicon | 0.25-2.0 | 0.25-2.0 | 0.25-2.0 | 0.5-2.2 |
| Dioxide | | | | |
| Sodium Lauryl | 0.0-2 | 0.5-2 | 0.0-2.0 | 0.0-2.0 |
| Sulfate | | | | |
| Sodium Stearyl | 0.2-1.5 | 0.1-0.5 | 0.25-1.0 | 0.1-0.5 |
| Fumarate | | | | |

wherein Spray Dried Intermediate comprises a spray-dried solid comprising a 1.5:1 to 1:1.5 w/w ratio of Compound 1: HPMC-AS (hydroxypropylmethylcellulose-acetate, succinate).

50. The pharmaceutical composition, formulation, or unit dosage form of any one of claims 40-47, comprising one of the following % w/w of ingredients shown below:

| | 25 mg | | 50 mg |
|---|---|---|---|
| Ingredients | w/w % | w/w % | w/w % |
| Spray Dried Intermediate | 23.0-52.0 | 16.0-66.0 | 20.0-80.0 |
| Microcrystalline | 0-70.0 or | 0-60.0 or | 0-58.0 or |
| Cellulose | 17.0-70.0 | 16.5-60.0 | 12.5-58.0 |
| Mannitol | 12.5-51.0 | 12.0-48.0 | 8.0-38.0 |
| Croscarmellose Sodium | 4.9-20.0 | 4.0-16.0 | 4.9-20.0 |
| Colloidal Silicon Dioxide | 0.5-2.0 | 0.5-2.0 | 0.5-2.0 |
| Sodium Lauryl Sulfate | 0.5-2.0 | 0.0-2.0 | 0.5-2.0 |
| Sodium Stearyl Fumarate | 0.3-1.5 | 0.3-1.5 | 0.3-1.5 |

wherein Spray Dried Intermediate comprises a spray-dried solid comprising a 1.5:1 to 1:1.5 w/w ratio of Compound 1: HPMC-AS (hydroxypropylmethylcellulose-acetate, succinate).

51. A method of producing a pharmaceutical composition, formulation, or unit dosage form, the method comprising wet granulation of a mixture comprising Compound 1: 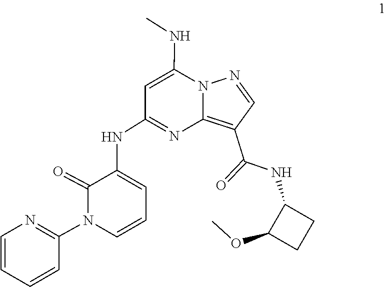 [SMILES: CNc1cc(Nc2cccn(-c3ccccn3)c2=O)nc2c(C(=O)N[C@@H]3CC[C@H]3OC)cnn12]
or a solvate or a pharmaceutically acceptable salt thereof;
and one or more solubilizers.

52. The method of claim 51, wherein the mixture further comprises a filler or diluent and a binder.

53. The method of claim 51 or 52, wherein the solubilizer comprises one or more solubilizers of formula I-A: 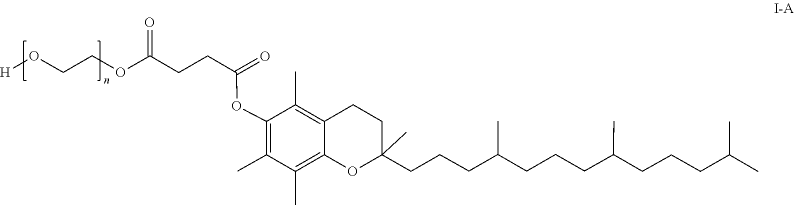
or a pharmaceutically acceptable salt thereof, wherein n is between about 10 to about 50, about 50 to about 100, about 100 to about 200, about 200 to about 500, about 500 to about 1,000, about 1,000 to about 2,000, about 2,000 to about 5,000, or about 5,000 to about 10,000.

54. The method of any one of claims 51-53, wherein the solubilizer comprises Vitamin E TPGS.

55. The method of any one of claims 51-54, wherein the filler or diluent comprises microcrystalline cellulose.

56. The method of any one of claims 51-55, wherein the filler or diluent comprises Avicel PH101.

57. The method of any one of claims 55-56, wherein the binder comprises povidone.

58. The method of any one of claims 49-51, wherein the binder comprises PVP K30.

59. The method of any one of claims 51-58, wherein the method comprises:
(1) wet granulation of the mixture; and
(2) drying the mixture.

60. The method of any one of claims 51-59, wherein the method comprises:
(1) wet granulation of the mixture;
(2) drying the mixture; and
(3) dry milling the mixture.

61. The method of any one of claims 51-60, wherein the method comprises:
(1) wet granulation of the mixture;
(2) drying the mixture;
(3) dry milling the mixture; and
(4) blending the mixture.

62. The method of any one of claims 51-61, wherein the method comprises:
(1) wet granulation of the mixture;
(2) drying the mixture;
(3) dry milling the mixture;
(4) blending the mixture; and
(5) encapsulating the mixture.

63. The method of any one of claims 51-62, wherein the mixture is de-lumped prior to wet granulation.

64. A method of manufacturing a formulation of any one of claims 40-50, comprising the steps of:
(1) providing an SDD (spray-dried dispersion) comprising Compound 1 or a solvate or a pharmaceutically acceptable salt thereof and a cellulosic polymer;
(2) blending the SDD with intragranular excipients;
(3) delumping the mixture;
(4) blending the mixture;
(5) dry granulating the mixture;
(6) blending the dry granulate with extragranular excipients; and
(7) optionally, filling the formulation into capsules.

65. The method of claim 64, wherein the SDD is prepared as follows:
(1) providing a solution of Compound 1 or a pharmaceutically acceptable salt thereof and the cellulosic polymer;
(2) filtering the solution;
(3) spray drying the solution to produce an SDJ (spray dried intermediate); and
(4) optionally, drying the SDI by secondary tray drying;
thus providing the SDD.
